(19) United States
(12) Patent Application Publication
(10) Pub. No.: US 2003/0054345 A1
(43) Pub. Date: Mar. 20, 2003

(54) Compositions and methods for the diagnosis and treatment of neuropsychiatric disorders, including schizophrenia

(75) Inventors: Joanne M. Meyer, Framingham, MA (US); Rory Barrington-Martin, Wayland, MA (US); Alexander Parker, Natick, MA (US); Glenn T. Barnes, Haverhill, MA (US)

Correspondence Address: DARBY & DARBY P.C., 805 Third Avenue, New York, NY, 10022, US

(73) Assignee: Millennium Pharmaceuticals, Inc.

(21) Appl. No.: 09/770,107

(22) Filed: Jan. 24, 2001

(51) Int. Cl. C12Q 1/68; C07H 21/04; C12P 19/34

(52) U.S. Cl. 435/6; 435/91.2; 536/24.3

(57) ABSTRACT

This invention relates to methods and compositions for diagnosing and treating neuropsychiatric disorders, such as schizophrenia, schizoaffective disorder, bipolar affective disorder, unipolar affective disorder and adolescent conduct disorder. In particular, the invention provides novel variants of DISC1 and DISC2 nucleic acid sequences, as well as novel DISC1 polypeptides encoded by these variant nucleic acid sequences. The variant DISC1 and DISC2 nucleic acid sequences provided by this invention, and the variant gene products they encode, are ones that correlate with the presence of a neuropsychiatric disorder in individuals. The invention, therefore, also provides methods and compositions for using the variant nucleic acids and polypeptides to diagnose and treat such neuropsychiatric disorders in individuals.

Numerous references are cited and discussed in the description of the invention, including patents, patent applications and various publications, as well as biological sequences (e.g., nucleic acid and polypeptide sequences) that are publicly available through various databases such as the GenBank database. The citation and/or discussion of such references is provided merely to clarify the description of the present invention and is not an admission that any such reference is “prior art” to the invention described herein. All references cited and discussed in this specification are incorporated herein by reference in their entirety and to the same extent as if each reference was individually incorporated by reference.

### 1. FIELD OF THE INVENTION

The present invention relates to compositions and methods which may be used to diagnose and treat neuropsychiatric disorders, including schizophrenia, schizoaffective disorder, bipolar disorder, unipolar affective disorder and adolescent conduct disorder.

The invention also relates to particular genes, referred to as DISC1 and DISC2, and their gene products. These genes are demonstrated herein to be associated with neuropsychiatric disorders (including schizophrenia, schizoaffective disorder, bipolar disorder, unipolar affective disorder and adolescent conduct disorder) and are therefore useful in the methods of the present invention.

The invention further relates to particular polymorphisms of the genes DISC1 and DISC2, including particular single nucleotide polymorphisms (SNPs). These novel polymorphisms are demonstrated here to co-segregate with neuropsychiatric disorders (particularly disorders such as schizophrenia, schizoaffective disorder, bipolar disorder, unipolar affective disorder and adolescent conduct disorder). The polymorphisms are useful, therefore, in novel methods of the invention for diagnosing and treating these disorders.

### 2. BACKGROUND OF THE INVENTION

There are only a few psychiatric disorders in which clinical manifestations of the disorder can be correlated with demonstrable defects in the structure and/or function of the nervous system. The vast majority of psychiatric disorders, however, involve subtle and/or undetectable changes and the cellular and molecular levels of nervous system structure and function. This lack of discernable neurological defects distinguishes “neuropsychiatric disorders” such as schizophrenia, attention deficit disorder (ADD), schizoaffective disorder, bipolar affective disorders and unipolar affective disorder from neurological disorders in which anatomical or biochemical pathologies are manifest. Hence, identification of causative defects in neuropathologies of neuropsychiatric disorders is needed so that clinicians may diagnose, evaluate and prescribe appropriate treatments for these disorders.

Schizophrenia is one example of a particularly serious and debilitating neuropsychiatric disorder that affects approximately 1% of the worldwide population. Currently, individuals may be evaluated for schizophrenia and other neuropsychiatric disorders using the criteria set forth in the most recent version of the American Psychiatric Association's Diagnostic and Statistical Manual of Mental Disorders (DSM-IV).

There is compelling evidence from family, twin and adoption studies for a significant genetic basis to schizophrenia and other neuropsychiatric disorders (McGuffin et al., Lancet 1995, 346:678-682). This has initiated searches directed towards identification of the genetic component or components of neuropsychiatric disorders using such methods as linkage analysis, association studies of candidate genes and mapping of cytogenetic abnormalities in psychiatric patients. However, while such techniques have been applied successfully to monogentic disorders, neuropsychiatric disorders apparently result from combined effects of multiple genes and environmental factors (see, McGuffin et al., supra). Such effects have complicated efforts to identify genetic components for these diseases.

Recent studies of a large Scottish family have identified a balanced (1;11)(q42.1;q14.3) translocation which segregates with major mental illness (see, St Clair et al., Lancet 1990, 336:13-16; Millar et al., Psychiatric Genet. 1998, 8:175-181). Sequencing of the chromosome 1 breakpoint of this translocation has revealed two genes, referred to as Disrupted-In-Schizophrenia 1 and 2, or DISC1 (GenBank Accession No. AF222980) and DISC2 (GenBank Accession No. AF222981), respectively. The genes DISC1 and DISC2 are directly disrupted by the translocation, suggesting that they should be considered formal candidate genes for susceptibility to neuropsychiatric disorders, particularly disorders such as schizophrenia, schizoaffective disorder, bipolar affective disorder, unipolar affective disorder and adolescent conduct disorder (Millar et al., Human Molecular Genetics 2000, 9:1415-1423).

It has been suggested, however, that the Scottish family in which DISC1 and DISC2 are disrupted may be atypical of other families and individuals affected by neuropsychiatric disorders such as schizophrenia. (see, Millar et al., Human Molecular Genetics 2000, 9:1415-1423). In particular, underlying genetic defects in DISC1 and DISC2 which are associated with neuropsychiatric disorders in other families or individuals are, most likely, different from the translocation observed for the particular Scottish family described by Millar et al.

There continues to exist, therefore, a need to identify specific genetic defects, mutations and polymorphisms that are associated with neuropsychiatric disorders such as schizophrenia, schizoaffective disorder, bipolar affective disorder, unipolar affective disorder and adolescent conduct disorder.

There further exists a need for compositions and methods to treat and/or diagnose these and other neuropsychiatric disorders, e.g., by identifying and correcting specific genetic defects, mutations and polymorphisms that are associated with such neuropsychiatric disorders.

There also continues to exist a need to identify specific genetic defects, mutations, and polymorphisms of the genes DISC1 and DISC2 that are associated with neuropsychiatric disorders such as schizophrenia, schizoaffective disorder, bipolar affective disorder, unipolar affective disorder and adolescent conduct disorder.

There further exists a need for compositions and methods to treat and/or diagnose these and other neuropsychiatric disorders using specific genetic defects, mutations and polymorphisms of the genes DISC1 and DISC2.

### 3. SUMMARY OF THE INVENTION

The present invention overcomes these and other problems in the art by providing novel compositions and methods that may be used to diagnose and treat a neuropsychiatric disorder such as, but not limited to, schizophrenia, schizoaffective disorder, bipolar affective disorder, unipolar affective disorder and adolescent conduct disorder. For example, in one embodiment the invention provides novel nucleic acid molecules which are associated with a neuropsychiatric disorder. These novel nucleic acid molecules are ones that correspond to genes known in the art as “Disrupted-in-Schizophrenia 1” (DISC1) and “Disrupted-in-Schizophrenia 2” (DISC2), respectively. The novel nucleic acid molecules of the invention are ones which contain one or more polymorphisms or variant nucleic acid sequences that are associated with a neuropsychiatric disorder such as schizophrenia, schizoaffective disorder, bipolar affective disorder, unipolar affective disorder and adolescent disorder. In preferred embodiments the one or more polymorphisms or variant nucleic acid sequences comprise one or more single nucleotide polymorphisms (SNPs).

For example, in one embodiment the invention provides a nucleic acid molecule comprising the nucleotide sequence set forth in FIGS. 1A-C (SEQ ID NO:1) and containing one or more single nucleotide polymorphisms. In one specific aspect of this embodiment, the nucleotide sequence comprises one or more of the nucleotide substitutions set forth in TABLE 5 shown in the Examples, infra. In another embodiment, the invention provides nucleic acid molecules comprising any of the nucleotide sequences set forth in TABLE 3 shown in the Examples, infra, and containing one or more single nucleotide polymorphisms. In a specific aspect of this embodiment, the nucleotide sequence comprises one or more of the nucleotide substitutions set forth in TABLE 5 shown in the Examples, infra.

The invention also provides gene products that are encoded by the DISC1 and DISC2 nucleic acid molecules of the invention. In one preferred embodiment, the invention provides a polypeptide having the amino acid sequence shown in FIG. 2 (SEQ ID NO:2) with one or more amino acid substitutions. For example, in specific aspects of this embodiment the polypeptide comprises the amino acid sequence shown in FIG. 2 (SEQ ID NO:2) with one or more of the specific amino acid substitutions set forth in TABLE 6B shown in the Examples, infra.

The invention additionally provides antibodies the specifically bind to a DISC1 or DISC2 gene product of the invention. For example, the invention provides antibodies that specifically bind to a polypeptide having the amino acid sequence shown in FIG. 2 (SEQ ID NO:2) and one or more of the specific amino acid substitutions set forth in TABLE 6B shown in the Examples, infra. In a particular, preferred aspect of this embodiment, the antibody is a monoclonal antibody.

In other embodiments, the invention provides novel methods for identifying individual who have a neuropsychiatric disorder or who have a propensity for a neuropsychiatric disorder. In one embodiment, the invention provides a method for identifying an individual who has a neuropsychiatric disorder or a propensity for a neuropsychiatric disorder by detecting, in a biological sample obtained from an individual, the presence of one the specific DISC1 or DISC2 nucleic acid molecules of the invention. Detection of the specific DISC1 or DISC2 nucleic acid molecule in the biological sample identifies the individual as one who has a neuropsychiatric disorder or who has a propensity for a neuropsychiatric disorder. In one exemplary aspect of this embodiment, the nucleic acid molecule detected is a nucleic acid molecule comprising the sequence set forth in FIGS. 1A-C (SEQ ID NO:1) and having one or more of the nucleotide substitutions set forth in TABLE 6A shown in the Examples, infra. In another exemplary aspect of this embodiment, the nucleic acid molecule detected is a nucleic acid molecule comprising a sequence selected from those set forth in TABLE 3 shown in the Examples, infra, and having one or more of the nucleotide substitutions set forth in TABLE 5 shown in the Examples, infra.

In an alternative embodiment, the invention provides a method for identifying an individual who has a neuropsychiatric disorder or a propensity for a neuropsychiatric disorder by detecting, in a biological sample obtained from an individual, the presence of a specific DISC1 or DISC2 gene product (e.g, a polypeptide) of the invention. Detection of the specific DISC1 or DISC2 gene product in the biological sample identifies the individual as one who has a neuropsychiatric disorder or who has a propensity for a neuropsychiatric disorder. In one particular aspect of this embodiment, the gene product detected is a polypeptide comprising the amino acid sequence set forth in FIG. 2 and having one or more of the amino acid substitutions set forth in TABLE 6B, shown in the Examples, infra. In other, exemplary, aspects of this embodiment, the polypeptide is detected by contacting, to the biological sample, an antibody that specifically binds to the polypeptide, and detecting the binding of this antibody to the polypeptide in the sample.

### 4. BRIEF DESCRIPTION OF THE DRAWINGS

FIGS. 1A-C sets forth the nucleotide sequence of the human DISC1 cDNA (SEQ ID NO:1) given in GenBank Accession No. AF222980.1 (GI:8163868).

FIG. 2 shows the predicted amino acid sequence (SEQ ID NO:2) encoded by the human DISC1 cDNA set forth in FIGS. 1A-C (SEQ ID NO:1). Amino acid residues of the predicted DISC1 polypeptide which are predicted to form α-helices are underlined, whereas a predicted globular domain of the DISC1 polypeptide is indicated by bold-faced type. Alternatively spliced amino acid residues of the DISC1 polypeptide are italicized.

FIGS. 3A-G sets forth the partial nucleotide sequence of the human DISC2 cDNA given in GenBank Accession No. AF222981 (GI:8163870) and presented, here, in SEQ ID NO:3.

FIG. 4 describes primer sequences (SEQ ID NOS: 44-127) for identifying and/or amplifying the DISC1 and/or DISC2 polymorphisms of the invention (see Example 3, infra).

### 5. DETAILED DESCRIPTION OF THE INVENTION

The present invention relates to a gene known as Disrupted-In-Schizophrenia 1 or DISC1 (GenBank Accession No. AF222980; GI:8163868) and to its gene product. The invention also relates to a gene known as Disrupted-In-Schizophrenia 2 or DISC2 (GenBank Accession No. AF222981; GI:8163870). Both DISC1 and DISC2 have been previously described. See, in particular, Millar et al., Human Molecular Genetics 2000, 9:1415-1423.

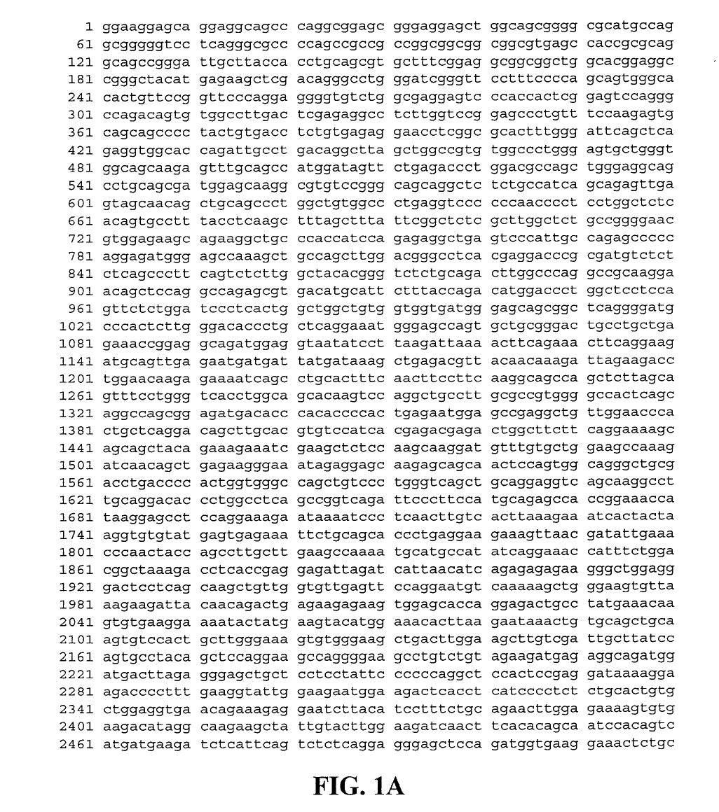

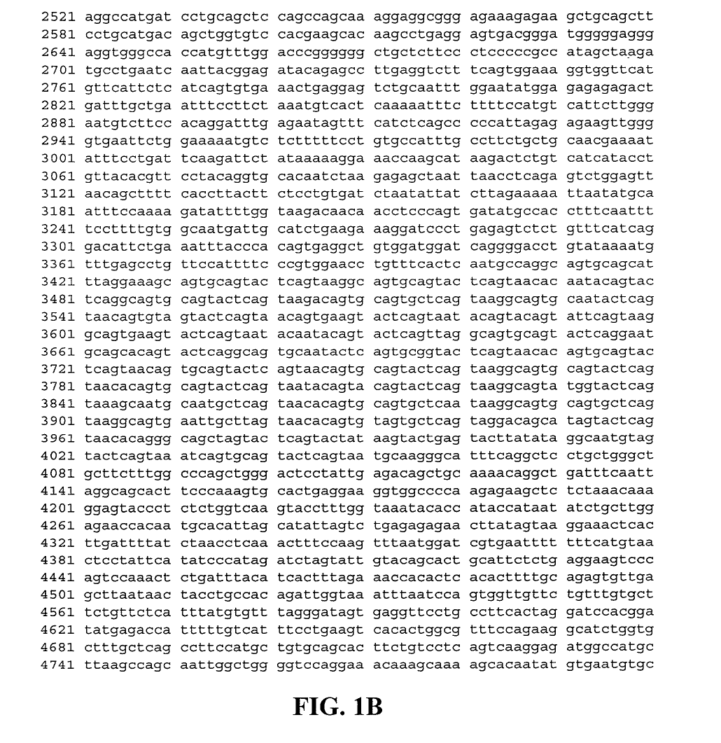

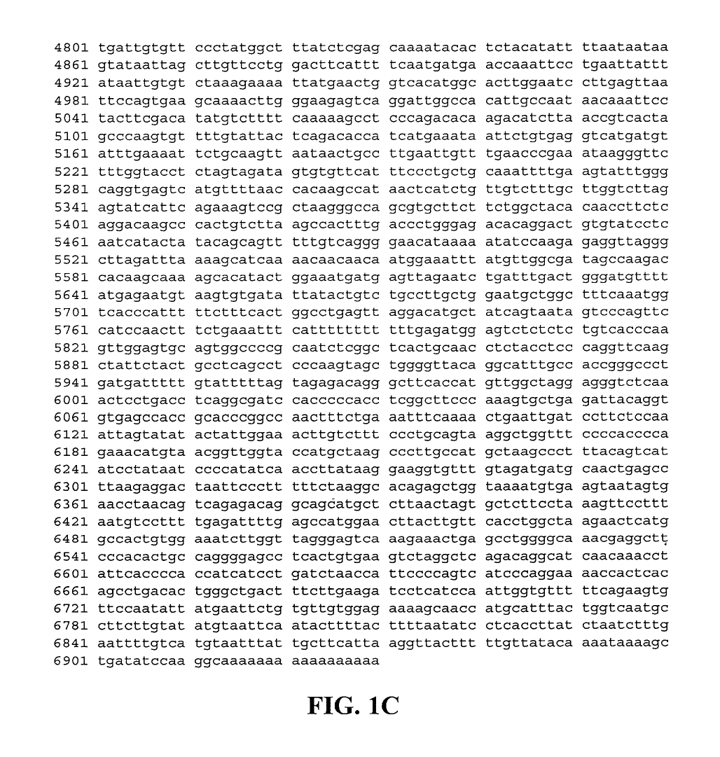

The DISC1 cDNA sequence (SEQ ID NO:1) is set forth here in FIGS. 1A-C. The complete cDNA sequence comprises a sequence of 6913 nucleotides (nucleotides 1-6913 of SEQ ID NO:1) and a poly(A) tail. The sequence further comprises a protein coding sequence 2565 nucleotides in length that begins with the ATG codon at nucleotide position 54 in FIGS. 1A-C (SEQ ID NO:1) and ends at the STOP codon located at position 2616 in FIGS. 1A-C (SEQ ID NO:1). The DISC1 cDNA sequence set forth in FIGS. 1A-C and in SEQ ID NO:1 also contains a 3′-untranslated region (UTR) of about 4294 nucleotides that begins after the STOP codon of the protein coding sequence (i.e., at about nucleotide position 2619 in SEQ ID NO:1). A common splice variant of the DISC1 cDNA sequence depicted in FIGS. 1A-C and in SEQ ID NO:1 is also known to exist. This splice variant comprises a sequence which is essentially identical to the DISC1 cDNA sequence set forth in FIGS. 1A-C and in SEQ ID NO:1, except that the nucleotide sequence corresponding to nucleotide positions 2295-2360 is omitted from this splice variant.

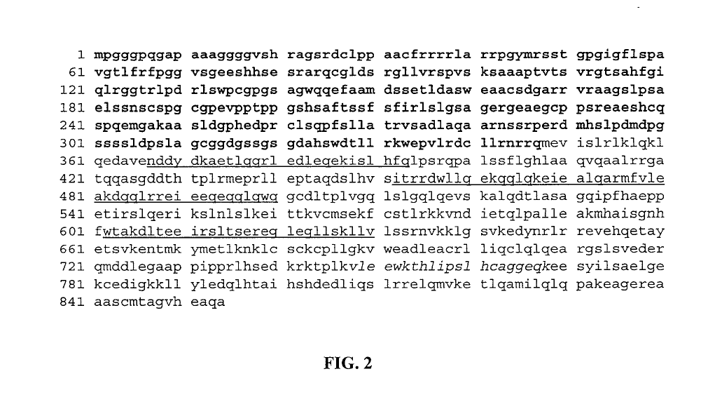

The coding sequence of the DISC1 cDNA set forth in FIGS. 1A-C and in SEQ ID NO:1 encodes a predicted DISC1 polypeptide having the amino acid sequence shown in FIG. 2 and set forth in SEQ ID NO:2. This DISC1 polypeptide may be divided into at least two regions of distinct secondary structure. The first region, which is also referred to as the N-terminal region, comprises approximately amino acid residues 1-347 of SEQ ID NO:1 and has been predicted to comprise at least one globular domain. The second region of secondary structure is also referred to as the C-terminal domain and comprises approximately amino acid residues 348-854 of SEQ ID NO:2. The C-terminal comprises at least three α-helix regions which are indicated by bold underlining in FIG. 2. These α-helices correspond to approximately amino acid residues 367-394, 452-500 and 602-630 of SEQ ID NO:2. It is understood that the α-helices contained in the DISC1 C-terminal region may interact with each other to form coiled coil structures. It is understood that the nucleotide and amino acid residue positions specified to delineate all regions and domains of the DISC1 gene and gene products are approximate.

As noted above, a splice variant of the DISC1 cDNA sequence set forth in FIGS. 1A-C (SEQ ID NO:1) is also known to exist. This DISC1 splice variant is predicted to encoded a polypeptide sequence that is essentially identical to the DISC1 polypeptide set forth in FIG. 2 (SEQ ID NO:2). However, the sequence corresponding to amino acid residues 748-768 of SEQ ID NO:2 is omitted from this DISC1 polypeptide splice variant.

The complete genomic DNA sequence for DISC1 has not been produced. However, the complete sequence may be partially represented by partial genomic sequences, provided herein in TABLE 3 (see, Examples, infra, and SEQ ID NO:4). The DISC1 genomic sequence comprises at least thirteen exons, which may be transcribed and spliced together to form a DISC1 mRNA. The boundaries of these exons/introns and their location in the partial genomic sequences are provided in TABLE 4 (see, Examples, infra, and SEQ ID NOS: 5-32).

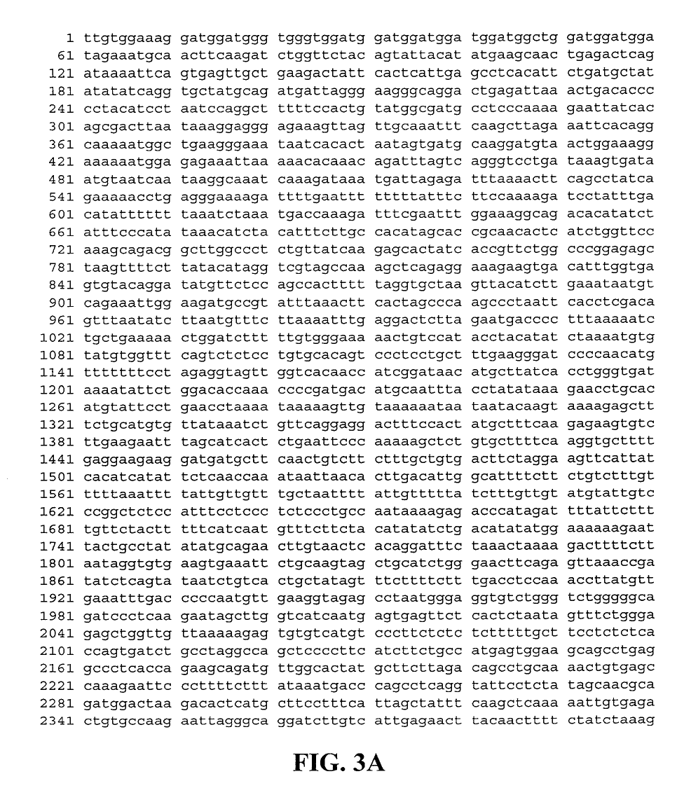

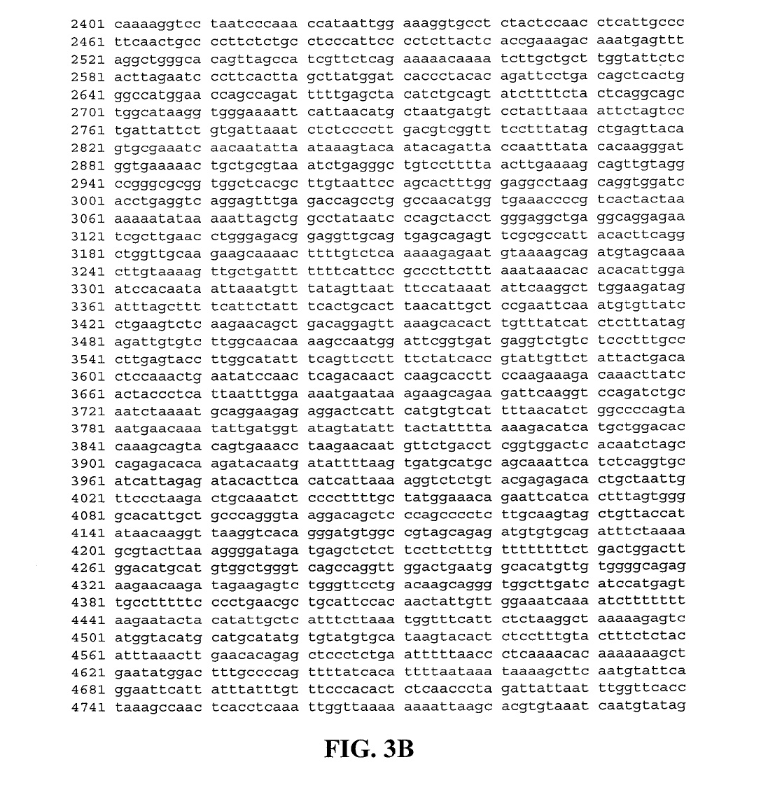

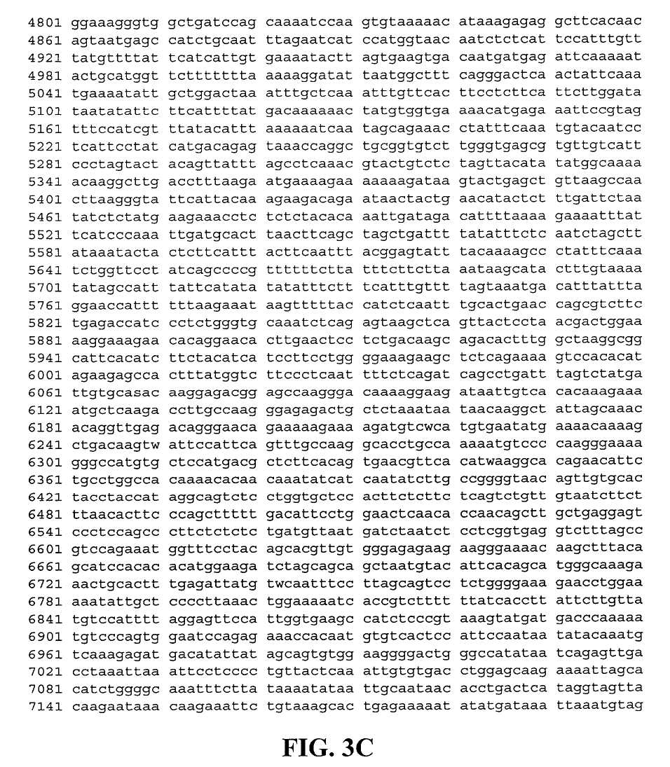

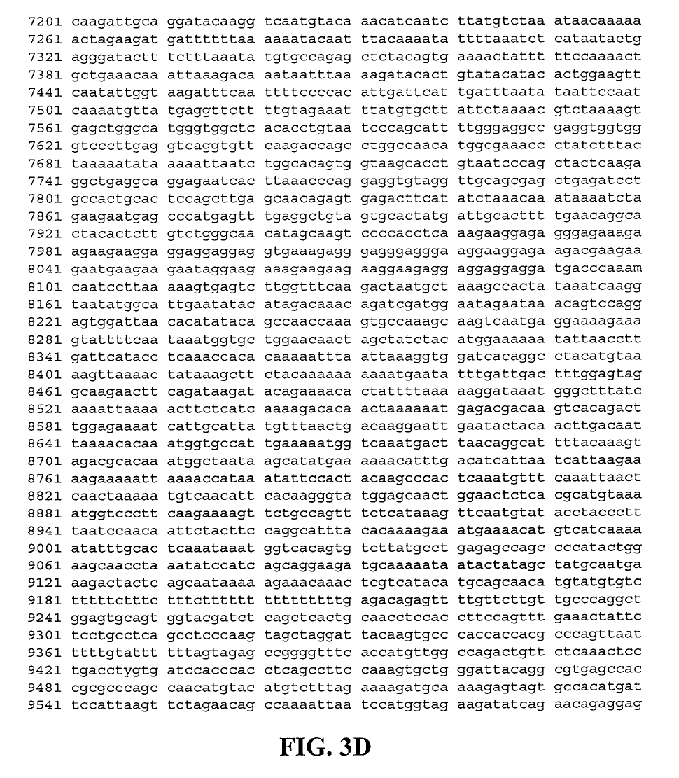

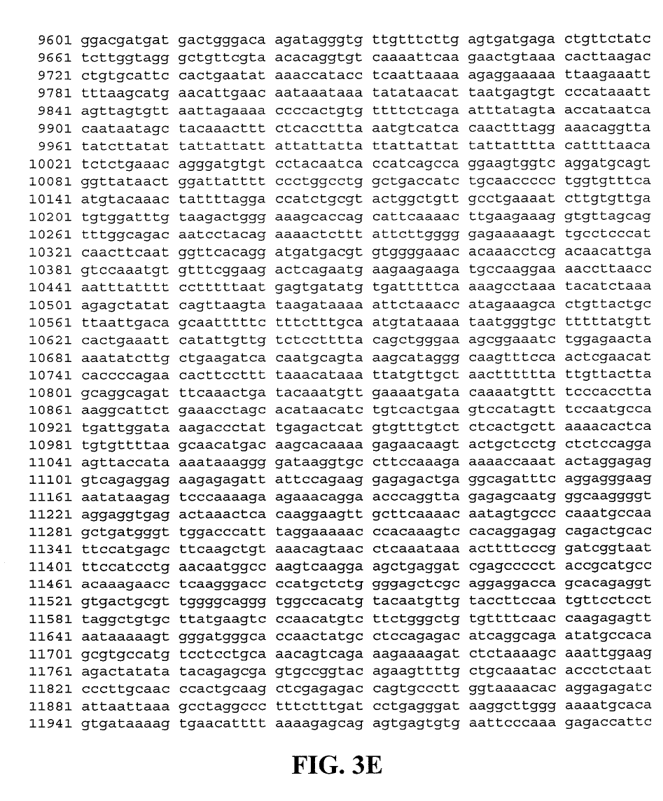

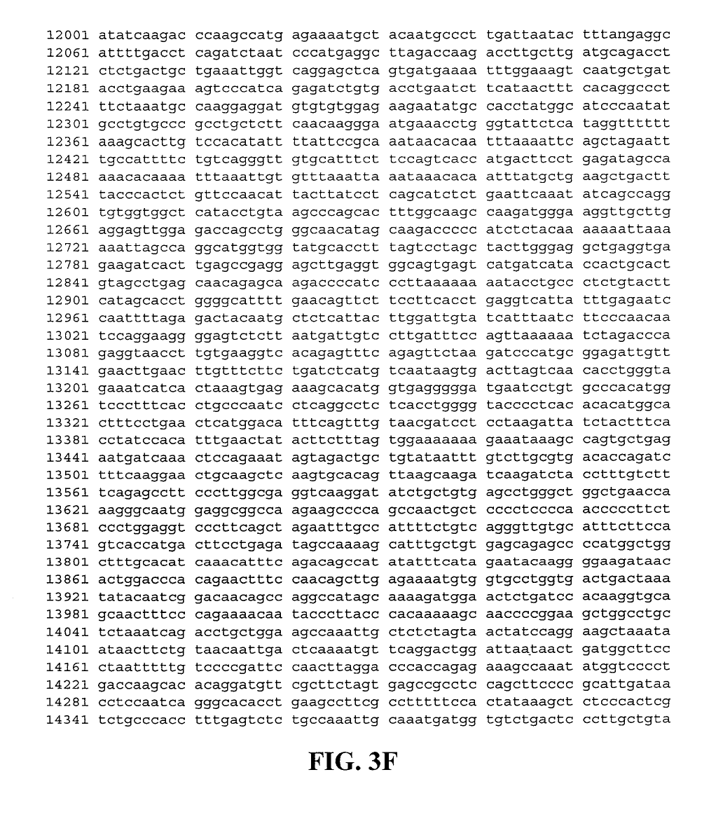

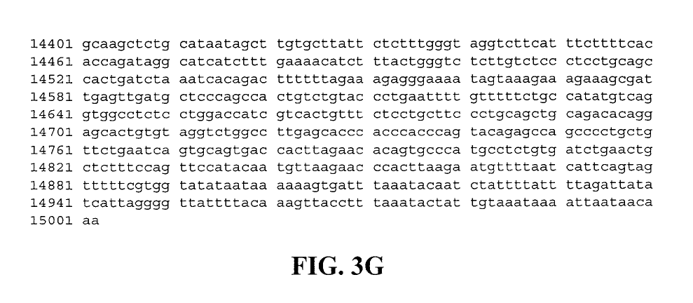

A partial DISC2 cDNA sequence (GenBank Accession No. AF222981, GI:8163870) is set forth in FIGS. 3A-G (SEQ ID NO:3). The genomic sequence of the human DISC2 gene is believed to overlap on human chromosome 1 with the sequence for DISC1, but is transcribed in the opposite direction. The DISC2 cDNA set forth in FIGS. 3A-G (SEQ ID NO:3) is not predicted to encode a DISC2 polypeptide or protein. Rather, DISC2 is thought to be a non-protein coding gene for a structural RNA.

The present invention relates, in particular, to novel variants of the DISC1 and DIS2 nucleic acids, including novel mutants, allelic variants and polymorphisms of full length DISC1 and DISC2 nucleic acids. Such variants include DISC1 and DISC2 nucleic acids which have one or more nucleic acid substitutions, insertions or deletions. In a preferred embodiment, the DISC1 and DISC2 nucleic acids of the invention are ones that have a single nucleotide polymorphism (SNP). Such SNPs include single base insertions, deletions and substitutions. In particularly preferred embodiments, the variant DISC1 and DISC2 nucleic acids of the invention are ones having a mutations or polymorphism which correlates with a neuropsychiatric disorder such as schizophrenia, schizoaffective disorder, bipolar disorder, unipolar affective disorder and adolescent conduct disorder. For example, preferred polymorphisms of the DISC1 and DISC2 nucleic acids of the invention are described in the Examples, infra, including ones that correlate to neuropsychiatric disorders such as schizophrenia, schizoaffective disorder, bipolar disorder, unipolar affective disorder and adolescent conduct disorder.

The present invention also relates to fragments of full length DISC1 and DISC2 nucleic acid sequences. For example, in preferred embodiments the DISC1 and DISC2 nucleic acid fragments of the invention comprise nucleotide sequences that correspond to at least 10 nucleotides, preferably at least 15 nucleotides, more preferably at least 20 nucleotides and still more preferably at least 25 nucleotides of a full length DISC1 or DISC2 nucleic acid sequence (e.g., of the DISC1 and DISC2 nucleic acid sequences set forth in FIGS. 1A-C and in FIGS. 3A-G, respectively, and in SEQ ID NOS:1 and 3). In other preferred embodiments, the DISC1 and DISC2 nucleic acid fragments of the invention comprise sequences of at least 10, preferably at least 15, more preferably at least 20 and still more preferably at least 25 nucleotides that hybridize, under conditions defined in detail below (e.g., for oligonucleotide molecules), to a full length DISC1 or DISC2 sequence (for example, to one of the DISC1 or DISC2 sequences set forth in FIGS. 1A-C and in FIGS. 3A-G, respectively, and in SEQ ID NOS:1 and 3) or to the complement of a full length DISC1 or DISC2 sequence.

Such DISC1 and DISC2 nucleic acid fragments may be used in the methods of the invention, e.g., to diagnose and/or treat a neuropsychiatric disorder such as schizophrenia, schizoaffective disorder, bipolar disorder, unipolar affective disorder and adolescent conduct disorder. Accordingly, in a preferred embodiment DISC1 and DISC2 nucleic acid fragments of the invention comprise nucleotide sequences of at least 10, at least 15, at least 20 or at least 25 nucleotides which comprise at least one polymorphic sequence of a DISC1 or DISC2 nucleic acid, such as one of the DISC1 or DISC2 polymorphic sequences described in the Examples, infra. In another preferred embodiment, DISC1 and DISC2 nucleic acid fragments of the invention may comprise nucleotide sequences of at least 10, at least 15, at least 20 or at least 25 nucleotides that specifically hybridize, under conditions defined in detail below (e.g., for oligonucleotides) to a DISC1 or DISC2 sequence (or its complement) that contains at least one polymorphism, such as one of the DISC1 or DISC2 polymorphisms described in the Examples, infra.

### 5.1 Definitions

The terms used in this specification generally have their ordinary meanings in the art, within the context of this invention and in the specific context where each term is used. Certain terms are discussed below, or elsewhere in the specification, to provide additional guidance to the practitioner in describing the devices and methods of the invention and how to make and use them.

### General definitions

The term “neuropsychiatric disorder”, which may also be referred to as a “major mental illness disorder” or “major mental illness”, refers to a disorder which may be generally characterized by one or more breakdowns in the adaptation process. Such disorders are therefore expressed primarily in abnormalities of thought, feeling and/or behavior producing either distress or impairment of function (i.e., impairment of mental function such with dementia or senility). Currently, individuals may be evaluated for various neuropsychiatric disorders using criteria set forth in the most recent version of the American Psychiatric Association's Diagnostic and Statistical Manual of Mental Health (DSM-IV).

Exemplary neuropsychiatric disorders include, but are not limited to, schizophrenia, attention deficit disorder (ADD), schizoaffective disorder, bipolar affective disorder, unipolar affective disorder, and adolescent conduct disorder.

As used herein, the term “isolated” means that the referenced material is removed from the environment in which it is normally found. Thus, an isolated biological material can be free of cellular components, i e., components of the cells in which the material is found or produced. In the case of nucleic acid molecules, an isolated nucleic acid includes a PCR product, an isolated mRNA, a cDNA, or a restriction fragment. In another embodiment, an isolated nucleic acid is preferably excised from the chromosome in which it may be found, and more preferably is no longer joined to non-regulatory, non-coding regions, or to other genes, located upstream or downstream of the gene contained by the isolated nucleic acid molecule when found in the chromosome. In yet another embodiment, the isolated nucleic acid lacks one or more introns. Isolated nucleic acid molecules include sequences inserted into plasmids, cosmids, artificial chromosomes, and the like. Thus, in a specific embodiment, a recombinant nucleic acid is an isolated nucleic acid. An isolated protein may be associated with other proteins or nucleic acids, or both, with which it associates in the cell, or with cellular membranes if it is a membrane-associated protein. An isolated organelle, cell, or tissue is removed from the anatomical site in which it is found in an organism. An isolated material may be, but need not be, purified.

The term “purified” as used herein refers to material that has been isolated under conditions that reduce or eliminate the presence of unrelated materials, i.e., contaminants, including native materials from which the material is obtained. For example, a purified protein is preferably substantially free of other proteins or nucleic acids with which it is associated in a cell; a purified nucleic acid molecule is preferably substantially free of proteins or other unrelated nucleic acid molecules with which it can be found within a cell. As used herein, the term “substantially free” is used operationally, in the context of analytical testing of the material. Preferably, purified material substantially free of contaminants is at least 50% pure; more preferably, at least 90% pure, and more preferably still at least 99% pure. Purity can be evaluated by chromatography, gel electrophoresis, immunoassay, composition analysis, biological assay, and other methods known in the art.

Methods for purification are well-known in the art. For example, nucleic acids can be purified by precipitation, chromatography (including preparative solid phase chromatography, oligonucleotide hybridization, and triple helix chromatography), ultracentrifugation, and other means. Polypeptides and proteins can be purified by various methods including, without limitation, preparative disc-gel electrophoresis, isoelectric focusing, HPLC, reversed-phase HPLC, gel filtration, ion exchange and partition chromatography, precipitation and salting-out chromatography, extraction, and countercurrent distribution. For some purposes, it is preferable to produce the polypeptide in a recombinant system in which the protein contains an additional sequence tag that facilitates purification, such as, but not limited to, a polyhistidine sequence, or a sequence that specifically binds to an antibody, such as FLAG and GST. The polypeptide can then be purified from a crude lysate of the host cell by chromatography on an appropriate solid-phase matrix. Alternatively, antibodies produced against the protein or against peptides derived therefrom can be used as purification reagents. Cells can be purified by various techniques, including centrifugation, matrix separation (e.g., nylon wool separation), panning and other immunoselection techniques, depletion (e.g. complement depletion of contaminating cells), and cell sorting (e.g., fluorescence activated cell sorting [FACS]). Other purification methods are possible. A purified material may contain less than about 50%, preferably less than about 75%, and most preferably less than about 90%, of the cellular components with which it was originally associated. The “substantially pure” indicates the highest degree of purity which can be achieved using conventional purification techniques known in the art.

A “sample” as used herein refers to a biological material which can be tested for the presence of DISC1 or DISC2 polypeptides, or for the presence of DISC1 or DISC2 nucleic acids, e.g., to evaluate a gene therapy or expression in a transgenic animal or to identify cells that express DISC1 or DISC2. Such samples can be obtained from any source, including tissue, blood and blood cells, including circulating hematopoietic stem cells (for possible detection of protein or nucleic acids), plural effusions, cerebrospinal fluid (CSF), ascites fluid, and cell culture. In a preferred embodiment, samples are obtained from brain tissue or from other tissues of the nervous system.

Non-human animals include, without limitation, laboratory animals such as mice, rats, rabbits, hamsters, guinea pigs, etc.; domestic animals such as dogs and cats; and, farm animals such as sheep, goats, pigs, horses, and cows, and especially such animals made transgenic with human DISC1 or DISC2.

In preferred embodiments, the terms “about” and “approximately” shall generally mean an acceptable degree of error for the quantity measured given the nature or precision of the measurements. Typical, exemplary degrees of error are within 20 percent (%), preferably within 10%, and more preferably within 5% of a given value or range of values. Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

The term “aberrant” or “abnormal”, as applied herein refers to an activity or feature which differs from (a) a normal or activity or feature, or (b) an activity or feature which is within normal variations of a standard value.

For example, an “abnormal” activity of a gene or protein such as the DISC1 or DISC2 gene or protein refers to an activity which differs from the activity of the wild-type or native gene or protein, or which differs from the activity of the gene or protein in a healthy subject, e.g., a subject not afflicted with a disease associated with a specific allelic variant of a DISC1 or DISC2 polymorphism. An activity of a gene includes, for instance, the transcriptional activity of the gene which may result from, e.g., an aberrant promoter activity. Such an abnormal transcriptional activity can result, e.g., from one or more mutations in a promoter region, such as in a regulatory element thereof. An abnormal transcriptional activity can also result from a mutation in a transcription factor involved in the control of gene expression.

An activity of a protein can be aberrant because it is stronger than the activity of its native counterpart. Alternatively, an activity can be aberrant because it is weaker or absent related to the activity of its native counterpart. An aberrant activity can also be a change in an activity. For example an aberrant protein can interact with a different protein relative to its native counterpart. A cell can have an aberrant activity due to overexpression or underexpression of the gene encoding DISC1 or DISC2. An aberrant DISC1 or DISC2 activity can result, e.g., from a mutation in the gene, which results, e.g., in lower or higher binding affinity of a ligand or substrate to the protein encoded by the mutated gene.

The term “molecule” means any distinct or distinguishable structural unit of matter comprising one or more atoms, and includes, for example, polypeptides and polynucleotides.

### Molecular Biology Definitions

In accordance with the present invention, there may be employed conventional molecular biology, microbiology and recombinant DNA techniques within the skill of the art. Such techniques are explained fully in the literature. See, for example, Sambrook, Fitsch & Maniatis, Molecular Cloning: A Laboratory Manual, Second Edition (1989) Cold Spring Harbor Laboratory Press, Cold Spring Harbor, N.Y. (referred to herein as “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins, eds. 1984); Animal Cell Culture (R. I. Freshney, ed. 1986); Immobilized Cells and Enzymes (IRL Press, 1986); B. E. Perbal, A Practical Guide to Molecular Cloning (1984); F. M. Ausubel et al. (eds.), Current Protocols in Molecular Biology, John Wiley & Sons, Inc. (1994).

The term “polymer” means any substance or compound that is composed of two or more building blocks (‘mers’) that are repetitively linked together. For example, a “dimer” is a compound in which two building blocks have been joined togther; a “trimer” is a compound in which three building blocks have been joined together; etc.

The term “polynucleotide” or “nucleic acid molecule” as used herein refers to a polymeric molecule having a backbone that supports bases capable of hydrogen bonding to typical polynucleotides, wherein the polymer backbone presents the bases in a manner to permit such hydrogen bonding in a specific fashion between the polymeric molecule and a typical polynucleotide (e.g., single-stranded DNA). Such bases are typically inosine, adenosine, guanosine, cytosine, uracil and thymidine. Polymeric molecules include “double stranded” and “single stranded” DNA and RNA, as well as backbone modifications thereof (for example, methylphosphonate linkages).

Thus, a “polynucleotide” or “nucleic acid” sequence is a series of nucleotide bases (also called “nucleotides”), generally in DNA and RNA, and means any chain of two or more nucleotides. A nucleotide sequence frequently carries genetic information, including the information used by cellular machinery to make proteins and enzymes. The terms include genomic DNA, cDNA, RNA, any synthetic and genetically manipulated polynucleotide, and both sense and antisense polynucleotides. This includes single- and double-stranded molecules; i.e., DNA-DNA, DNA-RNA, and RNA-RNA hybrids as well as “protein nucleic acids” (PNA) formed by conjugating bases to an amino acid backbone. This also includes nucleic acids containing modified bases, for example, thio-uracil, thio-guanine and fluoro-uracil.

The polynucleotides herein may be flanked by natural regulatory sequences, or may be associated with heterologous sequences, including promoters, enhancers, response elements, signal sequences, polyadenylation sequences, introns, 5′- and 3′-non-coding regions and the like. The nucleic acids may also be modified by many means known in the art. Non-limiting examples of such modifications include methylation, “caps”, substitution of one or more of the naturally occurring nucleotides with an analog, and internucleotide modifications such as, for example, those with uncharged linkages (e.g., methyl phosphonates, phosphotriesters, phosphoroamidates, carbamates, etc.) and with charged linkages (e.g., phosphorothioates, phosphorodithioates, etc.). Polynucleotides may contain one or more additional covalently linked moieties, such as proteins (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.) and alkylators to name a few. The polynucleotides may be derivatized by formation of a methyl or ethyl phosphotriester or an alkyl phosphoramidite linkage. Furthermore, the polynucleotides herein may also be modified with a label capable of providing a detectable signal, either directly or indirectly. Exemplary labels include radioisotopes, fluorescent molecules, biotin and the like. Other non-limiting examples of modification which may be made are provided, below, in the description of the present invention.

A “polypeptide” is a chain of chemical building blocks called amino acids that are linked together by chemical bonds called “peptide bonds”. The term “protein” refers to polypeptides that contain the amino acid residues encoded by a gene or by a nucleic acid molecule (e.g., an mRNA or a cDNA) transcribed from that gene either directly or indirectly. Optionally, a protein may lack certain amino acid residues that are encoded by a gene or by an mRNA. For example, a gene or mRNA molecule may encode a sequence of amino acid residues on the N-terminus of a protein (i.e., a signal sequence) that is cleaved from, and therefore may not be part of, the final protein. A protein or polypeptide, including an enzyme, may be a “native” or “wild-type”, meaning that it occurs in nature; or it may be a “mutant”, “variant” or “modified”, meaning that it has been made, altered, derived, or is in some way different or changed from a native protein or from another mutant.

A “ligand” is, broadly speaking, any molecule that binds to another molecule. In preferred embodiments, the ligand is either a soluble molecule or the smaller of the two molecules or both. The other molecule is referred to as a “receptor”. In preferred embodiments, both a ligand and its receptor are molecules (preferably proteins or polypeptides) produced by cells. In particularly preferred embodiments, a ligand is a soluble molecule and the receptor is an integral membrane protein (i.e., a protein expressed on the surface of a cell). However, the distinction between which molecule is the ligand and which is the receptor may be an arbitrary one.

The binding of a ligand to its receptor is frequently a step in signal transduction within a cell. Exemplary ligand-receptor interactions include, but are not limited to, binding of a hormone to a hormone receptor (for example, the binding of estrogen to the estrogen receptor) and the binding of a neurotransmitter to a receptor on the surface of a neuron.

“Amplification” of a polynucleotide, as used herein, denotes the use of polymerase chain reaction (PCR) to increase the concentration of a particular DNA sequence within a mixture of DNA sequences. For a description of PCR see Saiki et al., Science 1988, 239:487.

“Chemical sequencing” of DNA denotes methods such as that of Maxam and Gilbert (Maxam-Gilbert sequencing; see Maxam & Gilbert, Proc. Natl. Acad. Sci. U.S.A. 1977, 74:560), in which DNA is cleaved using individual base-specific reactions.

“Enzymatic sequencing” of DNA denotes methods such as that of Sanger (Sanger et al., Proc. Natl. Acad. Sci. U.S.A. 1977, 74:5463) and variations thereof well known in the art, in a single-stranded DNA is copied and randomly terminated using DNA polymerase.

A “gene” is a sequence of nucleotides which code for a functional “gene product”. Generally, a gene product is a functional protein. However, a gene product can also be another type of molecule in a cell, such as an RNA (e.g., a tRNA or a rRNA). For example, the gene DISC2, described supra, is not known to encode any particular protein and, rather, is believed encode a structural RNA molecule instead (see Millar et al., Human Molecular Genetics 2000, 9:1415-1423). For the purposes of the present invention, a gene also refers to an mRNA sequence which may be found in a cell. For example, measuring gene expression levels according to the invention may correspond to measuring mRNA levels. A gene may also comprise regulatory (i.e., non-coding) sequences as well as coding sequences. Exemplary regulatory sequences include promoter sequences, which determine, for example, the conditions under which the gene is expressed. The transcribed region of the gene may also include untranslated regions including introns, a 5′-untranslated region (5′-UTR) and a 3′-untranslated region (3′-UTR).

A “coding sequence” or a sequence “encoding” and expression product, such as a RNA, polypeptide, protein or enzyme, is a nucleotide sequence that, when expressed, results in the production of that RNA, polypeptide, protein or enzyme; i.e., the nucleotide sequence “encodes” that RNA or it encodes the amino acid sequence for that polypeptide, protein or enzyme.

A “promoter sequence” is a DNA regulatory region capable of binding RNA polymerase in a cell and initiation transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently found, for example, by mapping with nuclease S1), as well as protein binding domains (consensus sequences) responsible for the binding of RNA polymerase.

A coding sequence is “under the control of” or is “operatively associated with” transcriptional and translational control sequences in a cell when RNA polymerase transcribes the coding sequence into RNA, which is then trans-RNA spliced (if it contains introns) and, if the sequence encodes a protein, is translated into that protein.

The term “express” and “expression” means allowing or causing the information in a gene or DNA sequence to become manifest, for example producing RNA (such as rRNA or mRNA) or a protein by activating the cellular functions involved in transcription and translation of a corresponding gene or DNA sequence. A DNA sequence is expressed by a cell to form an “expression product” such as an RNA (e.g., a mRNA or a rRNA) or a protein. The expression product itself, e.g., the resulting RNA or protein, may also said to be “expressed” by the cell.

The term “transfection” means the introduction of a foreign nucleic acid into a cell. The term “transformation” means the introduction of a “foreign” (i.e., extrinsic or extracellular) gene, DNA or RNA sequence into a host cell so that the host cell will express the introduced gene or sequence to produce a desired substance, in this invention typically an RNA coded by the introduced gene or sequence, but also a protein or an enzyme coded by the introduced gene or sequence. The introduced gene or sequence may also be called a “cloned” or “foreign” gene or sequence, may include regulatory or control sequences (e.g., start, stop, promoter, signal, secretion or other sequences used by a cell's genetic machinery). The gene or sequence may include nonfunctional sequences or sequences with no known function. A host cell that receives and expresses introduced DNA or RNA has been “transformed” and is a “transformant” or a “clone”. The DNA or RNA introduced to a host cell can come from any source, including cells of the same genus or species as the host cell or cells of a different genus or species.

The terms “vector”, “cloning vector” and “expression vector” mean the vehicle by which a DNA or RNA sequence (e.g., a foreign gene) can be introduced into a host cell so as to transform the host and promote expression (e.g., transcription and translation) of the introduced sequence. Vectors may include plasmids, phages, viruses, etc and are discussed in greater detail below.

A “cassette” refers to a DNA coding sequence or segment of DNA that codes for an expression product that can be inserted into a vector at defined restriction sites. The cassette restriction sites are designed to ensure insertion of the cassette in the proper reading frame. Generally, foreign DNA is inserted at one or more restriction sites of the vector DNA, and then is carried by the vector into a host cell along with the transmissible vector DNA. A segment or sequence of DNA having inserted or added DNA, such as an expression vector, can also be called a “DNA construct.” A common type of vector is a “plasmid”, which generally is a self-contained molecule of double-stranded DNA, usually of bacterial origin, that can readily accept additional (foreign) DNA and which can readily introduced into a suitable host cell. A large number of vectors, including plasmid and fungal vectors, have been described for replication and/or expression in a variety of eukaryotic and prokaryotic hosts.

The term “host cell” means any cell of any organism that is selected, modified, transformed, grown or used or manipulated in any way for the production of a substance by the cell. For example, a host cell may be one that is manipulated to express a particular gene, a DNA or RNA sequence, a protein or an enzyme. Host cells can further be used for screening or other assays that are described infra. Host cells may be cultured in vitro or one or more cells in a non-human animal (e.g., a transgenic animal or a transiently transfected animal).

The term “expression system” means a host cell and compatible vector under suitable conditions, e.g. for the expression of a protein coded for by foreign DNA carried by the vector and introduced to the host cell. Common expression systems include E. coli host cells and plasmid vectors, insect host cells such as Sf9, Hi5 or S2 cells and Baculovirus vectors, Drosophila cells (Schneider cells) and expression systems, and mammalian host cells and vectors. For example, DISC1 and/or DISC2 may be expressed in PC12, COS-1, or C₂C₁₂ cells. Other suitable cells include CHO cells, HeLa cells, 293T (human kidney cells), mouse primary myoblasts, and NIH 3T3 cells.

The term “heterologous” refers to a combination of elements not naturally occurring. For example, the present invention includes chimeric RNA molecules that comprise an rRNA sequence and a heterologous RNA sequence which is not part of the rRNA sequence. In this context, the heterologous RNA sequence refers to an RNA sequence that is not naturally located within the ribosomal RNA sequence. Alternatively, the heterologous RNA sequence may be naturally located within the ribosomal RNA sequence, but is found at a location in the rRNA sequence where it does not naturally occur. As another example, heterologous DNA refers to DNA that is not naturally located in the cell, or in a chromosomal site of the cell. Preferably, heterologous DNA includes a gene foreign to the cell. A heterologous expression regulatory element is a regulatory element operatively associated with a different gene that the one it is operatively associated with in nature.

An “allele” refers to any one of a series of two or more genes that occupy the same position or locus on a chromosome. Generally, alleles refer to different forms of a gene that differ by at least one nucleic acid residue. Thus, as used here, the terms “allele” and “allelic variant” refer, not only to different forms of genomic sequences, but may also refer to different forms of sequences that are encoded by or otherwise derived from allelic variants of the genomic sequence. For example, the term allelic variant may refer to mRNA sequences that are encoded by allelic variants of a genomic sequence, or to cDNA sequences that are derived from such variant mRNA sequences. As it is used herein, the term allelic variant can also refer to protein or polypeptides sequences which are derived from (e.g., encoded by) allelic variants of a particular gene.

Allelic variants are usually described by comparing their nucleotide or (in the case of variant polypeptides) amino acid sequences to a common “wild-type” or “reference” sequence. Thus, a “wild-type” or “reference” allele of a gene refers to that allele of a gene having a genomic sequence designated as the wild-type sequence and/or encoding a polypeptide having an amino acid sequence that is also designated as a wild-type sequence. The wild-type allele may be arbitrarily selected from any of the different alleles that may exist for a particular gene. However, the allele is most typically selected to be the allele which is most prevalent in a population of individuals. Thus, for example, the wild-type DISC1 and 2 genomic sequences have been arbitrarily selected, here, as the genomic sequence deposited in GenBank (Accession No. AF222980, GI No. 8163868; and AF222981, GI No. 8163870; DISC1 and 2, respectively) and set forth here in SEQ ID NOS:1 and 3.

The term “polymorphism” refers, generally, to the coexistence of more than one form of a gene (e.g., more than one allele) within a population of individuals. The different alleles may differ at one or more positions of their nucleic acid sequences, which are referred to herein as “polymorphic locuses”. When used herein to describe polypeptides that are encoded by different alleles of a gene, the term “polymorphic locus” also refers to the positions in an amino acid sequence that differ among variant polypeptides encoded by different alleles.

The polymorphisms of the present invention include “single nucleotide polymorphisms” (SNPs) and microsatellite repeats. The term SNP refers to a polymorphic site occupied by a single nucleotide, which is the site of variation between allelic sequences. Typically, the polymorphic site of an SNP is flanked by highly conserved sequences (e.g., sequences that vary in lees than 1/100 and, more preferably, in less than 1/1000 individuals in a population). The polymorphic locus of an SNP may be a single base deletion, a single base insertion, or a single base substitution. Single base substitutions are particularly preferred.

A “microsatellite repeat” or “microsatellite”, as the term is used herein, refers to a short sequence of repeating nucleotides within a nucleic acid. Typically, a microsatellite repeat comprises a repeating sequence of two (i.e., a dinucleotide repeat), three (i.e., a trinucleotide repeat), four (i.e., a tetranucleotide repeat) or five (i.e., a pentanucleotide repeat) nucleotides. Microsatellites of the invention therefore have the general formula (N₁, N₂, . . . Nᵢ)ₙ, wherein N represents a nucleic acid residue (e.g., adenine, thymine, cytosine or guanine), i represents the number of the last nucleotide in the microsatellite, and n represents the number of times the motif is repeated in the microsatellite locus. In one embodiment the number of nucleotides in a microsatellite motif (i) is about six, preferably between two and five, and more preferably two, three or four. The total number of repeats (n) in a microsatellite repeat may be, e.g., from one to about 60, preferably from 4 to 40, and more preferably from 10 to 30 when i=2; is preferably between about 4-25, and more preferably between about 6-22 when i=3; and is preferably between about 4-15, and more preferably between about 5-10 when i=4. A DISC1 and/or DISC2 nucleic acid of the invention may comprise any microsatellite repeat of the above general formula. However, the following motifs are particularly preferred: CA, TC, and, AATTG; as well as all complements and permutations of such motifs (for example, TG, GA, and CAATT.

The term “locus” refers to a specific position on a chromosome. For example, the locus of a DISC1 and/or DISC2 gene refers to the chromosomal position of that gene.

The term “linkage” refers to the tendency of genes, alleles, loci or genetic markers to be inherited together as a result of their location on the same chromosome. Linkage may be measured, e.g., by the percent recombination between two genes, alleles, loci or genetic markers.

The terms “mutant” and “mutation” mean any detectable change in genetic material, e.g., DNA, or any process, mechanism or result of such a change. This includes gene mutations, in which the structure (e.g., DNA sequence) of a gene is altered, any gene or DNA arising from any mutation process, and any expression product (e.g., RNA, protein or enzyme) expressed by a modified gene or DNA sequence. The term “variant” may also be used to indicate a modified or altered gene, DNA sequence, RNA, enzyme, cell, etc.; i.e., any kind of mutant. “Sequence-conservative variants” of a polynucleotide sequence are those in which a change of one or more nucleotides in a given codon position results in no alteration in the amino acid encoded at that position.

“Function-conservative variants” of a polypeptide or polynucleotide are those in which a given amino acid residue in the polypeptide, or the amino acid residue encoded by a codon of the polynucleotide, has been changed or altered without altering the overall conformation and function of the polypeptide. For example, function-conservative variants may include, but are not limited to, replacement of an amino acid with one having similar properties (for example, polarity, hydrogen bonding potential, acidic, basic, hydrophobic, aromatic and the like). Amino acid residues with similar properties are well known in the art. For example, the amino acid residues arginine, histidine and lysine are hydrophilic, basic amino acid residues and may therefore be interchangeable. Similar, the amino acid residue isoleucine, which is a hydrophobic amino acid residue, may be replaced with leucine, methionine or valine. Such changes are expected to have little or no effect on the apparent molecular weight or isoelectric point of the polypeptide. Amino acid residues other than those indicated as conserved may also differ in a protein or enzyme so that the percent protein or amino acid sequence similarity (e.g., percent identity or homology) between any two proteins of similar function may vary and may be, for example, from 70% to 99% as determined according to an alignment scheme such as the Cluster Method, wherein similarity is based on the MEGALIGN algorithm. “Function-conservative variants” of a given polypeptide also include polypeptides that have at least 60% amino acid sequence identity to the given polypeptide as determined, e.g., by the BLAST or FASTA algorithms. Preferably, function-conservative variants of a given polypeptide have at least 75%, more preferably at least 85% and still more preferably at least 90% amino acid sequence identity to the given polypeptide and, preferably, also have the same or substantially similar properties (e.g., of molecular weight and/or isoelectric point) or functions (e.g., biological functions or activities) as the native or parent polypeptide to which it is compared.

The term “homologous”, in all its grammatical forms and spelling variations, refers to the relationship between two proteins that possess a “common evolutionary origin”, including proteins from superfamilies (e.g., the immunoglobulin superfamily) in the same species of organism, as well as homologous proteins from different species of organism (for example, myosin light chain polypeptide, etc.; see, Reeck et al., Cell 1987, 50:667). Such proteins (and their encoding nucleic acids) have sequence homology, as reflected by their sequence similarity, whether in terms of percent identity or by the presence of specific residues or motifs and conserved positions.

The term “sequence similarity”, in ail its grammatical forms, refers to the degree of identity or correspondence between nucleic acid or amino acid sequences that may or may not share a common evolutionary origina (see, Reeck et al., supra). However, in common usage and in the instant application, the term “homologous”, when modified with an adverb such as “highly”, may refer to sequence similarity and may or may not relate to a common evolutionary origin.

In specific embodiments, two nucleic acid sequences are “substantially homologous” or “substantially similar” when at least about 80%, and more preferably at least about 90% or at least about 95% of the nucleotides match over a defined length of the nucleic acid sequences, as determined by a sequence comparison algorithm known such as BLAST, FASTA, DNA Strider, CLUSTAL, etc. An example of such a sequence is an allelic or species variant of the specific genes of the present invention. Sequences that are substantially homologous may also be identified by hybridization, e.g., in a Southern hybridization experiment under, e.g., stringent conditions as defined for that particular system.

Similarly, in particular embodiments of the invention, two amino acid sequences are “substantially homologous” or “substantially similar” when greater than 80% of the amino acid residues are identical, or when greater than about 90% of the amino acid residues are similar (i.e., are functionally identical). Preferably the similar or homologous polypeptide sequences are identified by alignment using, for example, the GCG (Genetics Computer Group, Program Manual for the GCG Package, Version 7, Madison Wis.) pileup program, or using any of the programs and algorithms described above (e.g., BLAST, FASTA, CLUSTAL, etc.).

As used herein, the term “oligonucleotide” refers to a nucleic acid, generally of at least 10, preferably at least 15, and more preferably at least 20 nucleotides, preferably no more than 100 nucleotides, that is hybridizable to a genomic DNA molecule, a cDNA molecule, or an mRNA molecule encoding a gene, mRNA, cDNA, or other nucleic acid of interest. Oligonucleotides can be labeled, e.g., with ³²P-nucleotides or nucleotides to which a label, such as biotin or a fluorescent dye (for example, Cy3 or Cy5) has been covalently conjugated. In one embodiment, a labeled oligonucleotide can be used as a probe to detect the presence of a nucleic acid. In another embodiment, oligonucleotides (one or both of which may be labeled) can be used as PCR primers, either for cloning full length or a fragment of DISC1 or DISC2, or to detect the presence of nucleic acids encoding DISC1 or DISC2. In a further embodiment, an oligonucleotide of the invention can form a triple helix with a DISC1 DNA molecule or with a DISC2 DNA molecule. Generally, oligonucleotides are prepared synthetically, preferably on a nucleic acid synthesizer. Accordingly, oligonucleotides can be prepared with non-naturally occurring phosphoester analog bonds, such as thioester bonds, etc.

The present invention provides antisense nucleic acids (including ribozymes), which may be used to inhibit expression of a DISC1 or a DISC2 gene or their respective gene products. An “antisense nucleic acid” is a single stranded nucleic acid molecule which, on hybridizing under cytoplasmic conditions with complementary bases in an RNA or DNA molecule, inhibits the latter's role. If the RNA is a messenger RNA transcript, the antisense nucleic acid is a countertranscript or mRNA-interfering complementary nucleic acid. As presently used, “antisense” broadly includes RNA-RNA interactions, RNA-DNA interactions, triple helix interactions, ribozymes and RNase-H mediated arrest. Antisense nucleic acid molecules can be encoded by a recombinant gene for expression in a cell (e.g., U.S. Pat. No. 5,814,500; U.S. Pat. No. 5,811,234), or alternatively they can be prepared synthetically (e.g., U.S. Pat. No. 5,780,607). Other specific examples of antisense nucleic acid molecules of the invention are provided infra.

Specific non-limiting examples of synthetic oligonucleotides envisioned for this invention include, in addition to the nucleic acid moieties described above, oligonucleotides that contain phosphorothioates, phosphotriesters, methyl phosphonates, short chain alkyl, or cycloalkyl intersugar linkages or short chain heteroatomic or heterocyclic intersugar linkages. Most preferred are those with CH₂—NH—O—CH₂, CH₂—N(CH₃)—O—CH₂, CH₂—O—N(CH₃)—CH₂, CH₂—N(CH₃)—N(CH₃)—CH₂ and O—N(CH₃)—CH₂—CH₂ backbones (where phosphodiester is O—PO₂—O—CH₂). U.S. Pat. No. 5,677,437 describes heteroaromatic olignucleoside linkages. Nitrogen linkers or groups containing nitrogen can also be used to prepare oligonucleotide mimics (U.S. Pat. Nos.5,792,844 and 5,783,682). U.S. Pat. No. 5,637,684 describes phosphoramidate and phosphorothioamidate oligomeric compounds. Also envisioned are oligonucleotides having morpholino backbone structures (U.S. Pat. No. 5,034,506). In other embodiments, such as the peptide-nucleic acid (PNA) backbone, the phosphodiester backbone of the oligonucleotide may be replaced with a polyamide backbone, the bases being bound directly or indirectly to the aza nitrogen atoms of the polyamide backbone (Nielsen et al., Science 254:11497, 1991). Other synthetic oligonucleotides may contain substituted sugar moieties comprising one of the following at the 2′ position: OH, SH, SCH₃, F, OCN, O(CH₂)ₙNH₂ or O(CH₂)ₙCH₃ where n is from 1 to about 10; C₁ to C₁₀ lower alkyl, substituted lower alkyl, alkaryl or aralkyl; Cl; Br; CN; CF₃; OCF₃; O—; S—, or N-alkyl; O—, S—, or N-alkenyl; SOCH₃; SO₂CH₃; ONO₂; NO₂; N₃; NH₂; heterocycloalkyl; heterocycloalkaryl; aminoalkylamino; polyalkylamino; substitued silyl; a fluorescein moiety; an RNA cleaving group; a reporter group; an intercalator; a group for improving the pharmacokinetic properties of an oligonucleotide; or a group for improving the pharmacodynamic properties of an oligonucleotide, and other substituents having similar properties. Oligonucleotides may also have sugar mimetics such as cyclobutyls or other carbocyclics in place of the pentofuranosyl group. Nucleotide units having nucleosides other than adenosine, cytidine, guanosine, thymidine and uridine, such as inosine, may be used in an oligonucleotide molecule.

A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength (see Sambrook et al., supra). The conditions of temperature and ionic strength determine the “stringency” of the hybridization. For preliminary screening for homologous nucleic acids, low stringency hybridization conditions, corresponding to a Tₘ (melting temperature) of 55° C., can be used, e.g., 5×SSC, 0.1% SDS, 0.25% milk, and no formamide; or 30% formamide, 5×SSC, 0.5% SDS). Moderate stringency hybridization conditions correspond to a higher Tₘ, e.g., 40% formamide, with 5× or 6×SCC. High stringency hybridization conditions correspond to the highest Tₘ, e.g., 50% formamide, 5× or 6×SCC. SCC is a 0.15M NaCl, 0.015M Na-citrate. Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tₘ for hybrids of nucleic acids having those sequences. The relative stability (corresponding to higher Tₘ) of nucleic acid hybridizations decreases in the following order: RNA:RNA, DNA:RNA, DNA:DNA. For hybrids of greater than 100 nucleotides in length, equations for calculating Tₘ have been derived (see Sambrook et al., supra, 9.50-9.51). For hybridization with shorter nucleic acids, i.e., oligonucleotides, the position of mismatches becomes more important, and the length of the oligonucleotide determines its specificity (see Sambrook et al., supra, 11.7-11.8). A minimum length for a hybridizable nucleic acid is at least about 10 nucleotides; preferably at least about 15 nucleotides; and more preferably the length is at least about 20 nucleotides.

In a specific embodiment, the term “standard hybridization conditions” refers to a Tₘ of 55° C., and utilizes conditions as set forth above. In a preferred embodiment, the Tₘ is 60° C.; in a more preferred embodiment, the Tₘ is 65° C. In a specific embodiment, “high stringency” refers to hybridization and/or washing conditions at 68° C. in 0.2×SSC, at 42° C. in 50% formamide, 4×SSC, or under conditions that afford levels of hybridization equivalent to those observed under either of these two conditions.

Suitable hybridization conditions for oligonucleotides (e.g., for oligonucleotide probes or primers) are typically somewhat different than for full-length nucleic acids (e.g., full-length cDNA), because of the oligonucleotides' lower melting temperature. Because the melting temperature of oligonucleotides will depend on the length of the oligonucleotide sequences involved, suitable hybridization temperatures will vary depending upon the oligoncucleotide molecules used. Exemplary temperatures may be 37° C. (for 14-base oligonucleotides), 48° C. (for 17-base oligoncucleotides), 55° C. (for 20-base oligonucleotides) and 60° C. (for 23-base oligonucleotides). Exemplary suitable hybridization conditions for oligonucleotides include washing in 6×SSC/0.05% sodium pyrophosphate, or other conditions that afford equivalent levels of hybridization.

### 5.2 DISC1 and DISC2 Nucleic Acids

The DISC1 and DISC2 nucleic acid molecules of the invention are defined above, and include DNA and RNA molecules as well as nucleic acid molecules comprising any of the modifications (e.g., modified bases and/or backbone) described supra.

### DISC1

With respect, first, to DISC1 nucleic acids, a DISC1 nucleic acid molecule comprises a nucleic acid sequence that encodes a DISC1 polypeptide (described in detail, infra), the complement of a nucleic acid sequence that encodes a DISC1 polypeptide, and fragments thereof. Thus, in one preferred embodiment a DISC1 nucleic acid molecules of the invention comprises nucleotide sequences that encode the amino acid sequence set forth in FIG. 2 (SEQ ID NO:2), such as the nucleotide sequence set forth in FIGS. 1A-C (SEQ ID NO:1) and, in particular, the nucleotide sequence of positions 54-2616 of the sequence set forth in FIGS. 1A-C (SEQ ID NO:1). In another preferred embodiment, a DISC1 nucleic acid molecule of the invention comprises a nucleic acid sequence that encodes a splice variant of the DISC1 polypeptide set forth in FIG. 2 and in SEQ ID NO:2. For example, a DISC1 nucleic acid molecule of the invention may encode a DISC1 polypeptide that is essentially identical to the DISC1 polypeptide sequence set forth in FIG. 2 (SEQ ID NO:2), except that the amino acid residues corresponding to positions 748-768 of SEQ ID NO:2 are omitted. In a preferred aspect of this embodiment, the DISC1 nucleic acid molecule comprises a coding sequence that is essentially identical to the coding sequence set forth in FIGS. 1A-C (SEQ ID NO:1) except that the sequence corresponding to nucleotide positions 2295-2360 is omitted.

Alternatively, a DISC1 nucleic acid molecule of the invention may encode one or more exons of a DISC1 gene product. For example, in one embodiment a DISC1 nucleic acid molecule of the invention may encode the sequence of amino acid residues 768-768 of the sequence set forth in FIG. 2 (SEQ ID NO:2). In a preferred aspect of the embodiment, the DISC1 nucleic acid molecule comprises the sequence of nucleotide positions 2295-2360 of the sequence set forth in FIGS. 1A-C (SEQ ID NO:1).

In other embodiments, DISC1 nucleic acid molecules of the invention comprise a nucleotide sequence that encodes one or more domains of a DISC1 polypeptide. For example, a DISC1 nucleic acid molecule of the invention may encode an N-terminal domain of a DISC1 polypeptide (e.g., amino acid residues 1-247 of SEQ ID NO:2), a C-terminal domain of a DISC1 polypeptide (e.g., amino acid residues 348-854 of SEQ ID NO:2), one or more α-helix domains of a DISC1 polypeptide (e.g., amino acid residues 367-394, 452-500 or 602-630 of SEQ ID NO:2), a coiled-coil domain of a DISC1 polypeptide (e.g., two or more α-helix domain of a DISC1 polypeptide and, optionally, the intervening amino acid residues; for example, in a preferred embodiment the coiled-coil domain comprises amino acid residues 367-630 of SEQ ID NO:2), or a globular domain of a DISC1 polypeptide.

In another, preferred embodiment, a DISC1 nucleic acid molecule of the invention is one which comprises a nucleotide sequence selected from the sequences set forth in TABLE 3 shown in the Examples, infra, or a portion thereof. For example, a DISC1 nucleic acid molecule of the invention may also be a nucleic acid molecule comprising the sequence for one or more introns indicated in the DISC1 genomic sequences set forth in TABLE 3; for one or more exons, delineated in TABLE 4, infra, of the DISC1 genomic sequences in TABLE 3; for the 5′-promoter region comprised in one or more of the genomic sequence set forth in TABLE 3, or a combination of one or more of the individual exons, introns and/or the 5′-promoter region from the DISC1 genomic sequence set forth in TABLE3.

In other embodiments, DISC1 nucleic acid molecules of the invention comprise a nucleotide sequence which encodes one or more domains of a DISC1 polypeptide. For example, a DISC1 nucleic acid molecule of the invention may encode an N-terminal domain of a DISC1 polypeptide (e.g., amino acid residues 1-347 of SEQ ID NO:2), a C-terminal domain of a DISC1 polypeptide (e.g., amino acid residues 348-854 of SEQ ID NO:2), one or more α-helix domains of a DISC1 polypeptide (e.g., amino acid residues 367-394, 452-500 or 602-630 of SEQ ID NO:2) or a globular domain of a DISC1 polypeptide.

The DISC1 nucleic acid molecules of the present invention may also comprise a nucleic acid sequence that encodes one or more fragments (e.g., an epitope) of a DISC1 polypeptide.

The DISC1 nucleic acid molecules of the present invention also include nucleic acid molecules that comprise modified or variant DISC1 nucleic acid sequences, including DISC1 nucleic acid molecules that have one or more nucleic acid insertions, substitutions, deletions or truncations. Such modified and variant DISC1 nucleic acid molecules includes ones that comprise coding sequences for modified DISC1 polypeptides (e.g., having amino acid substitutions, deletions or truncations) and for variants (including analogs and homologs from the same and different species of organism) of DISC1 polypeptides. In preferred embodiments, such nucleic acid molecules have at least 50%, preferably at least 75% and more preferably at least 90% sequence identity to a DISC1 coding nucleotide sequence such as the coding sequence comprising nucleotide positions 54 through 2616 of the sequence set forth in FIGS. 1A-C (SEQ ID NO:1).

Preferably, the modified or variant DISC1 nucleic acid molecules of the invention comprise sequence modifications or variations that correlate with a neuropsychiatric disorder, such as schizophrenia, schizoaffective disorder, bipolar affective disorder, unipolar affective disorder and adolescent conduct disorder. Particularly preferred variant DISC1 nucleic acids include those described in the Examples, infra.

As used here, the term “homologous”, in all its grammatical forms and spelling variations, refers to the relationship between nucleic acids, and the proteins or gene products they encode, that are understood to possess a “common evolutionary origina”, including genes and proteins from superfamilies (for example, the immunoglobulin superfamily) and homologous proteins and nucleic acids (e.g., genes) from different species. See, for example, Reeck et al., Cell 1987, 50:667. Corresponding nucleic acid molecules (e.g., genes) and proteins from different species are referred to as “orthologs.” Homologous and orthologous proteins, and their encoding nucleic acids, have sequence homology which is reflected by their sequence similarity. Such sequence similarity may be indicated, for example, by the percent of sequence similarity (e.g., a percentage of nucleotide or amino acid sequence identity or homology), or by the presence of certain nucleotides or certain amino acid residues and conserved positions, or by the presence of specific “sequence motifs” (i.e., specific sequences of nucleotides or amino acid residues) at conserved positions.

The term “sequence similarity”, in all its grammatical forms, refers to the degree of identity or correspondence between nucleotide or amino acid sequences. Except as otherwise noted herein, the term “homologous” refers merely to sequence similarity and does not necessarily relate to a common evolutionary origin In specific embodiments, two polypeptide sequences are “substantially homologous” or “substantially similar” when the polypeptides are at least 35-40% similar as determined by one of the algorithms disclosed herein. Preferably, two polypeptides are substantially homologous when the polypeptides are at least about 60%, and more preferably at least about 90 or 95% similar in one or more highly conserved domains or, for alleles, across the entire amino acid sequence. Two nucleic acid sequences are “substantially homologous” or “substantially similar” when the polypeptides they encode are substantially homologous, as defined above. Alternatively, two nucleic acid sequences may be “substantially homologous” or “substantially similar” if their nucleotide sequences are, themselves, at least about 35-40% similar, preferably at least about 60% similar and more preferably at least about 90 to 95% similar, as determined by one of the algorithms disclosed herein.

Sequence comparison algorithms that may be used to compare amino acid or nucleic acid sequences include the BLAST algorithms (e.g., BLASTP, BLASTN and BLASTX), FASTA, DNA Strider, the GCG (Genetics Computer Group, Program Manual for the GCG Package, Version 7, Madison, Wis.) pileup program, etc. Unless otherwise stated, all sequence comparisons referred to herein are done using the default parameters provided with these algorithms.

In one embodiment, the homologous DISC1 nucleic acids of the invention (including, e.g., DISC1 analogs or variants) encode functionally similar molecules (i.e., molecules that perform one or more DISC1 functions or have one or more DISC1 bioactivities). Thus, in a specific embodiment, an analog or variant of a DISC1 polypeptide is a function-conservative variant. “Function-conservative variants” of a polypeptide are those in which a given amino acid residue of the polypeptide has been changed without altering the overall conformation and/or function (e.g., bioactivity) of the polypeptide. Such changes include, but are not limited to, replacement of an amino acid with one having similar properties; such as similar properties of polarity, hydrogen bonding potential, acidity, alkalinity, hydrophobicity, aromaticity and the like. For example and not by way of limitation, arginine, histidine and lysine or hydrophilic-basic amino acids and may be interchangeable. Similarly, isoleucine, a hydrophobic amino acid, may be replaced with leucine, methionine or valine. Such changes are expected to have little or no effect on the apparent molecular weight or isoelectric point of the protein or polypeptide.

In another embodiment, the homologous DISC1 nucleic acids of the invention (including, e.g., DISC1 analogs or variants) are sequence conservative variants. “Sequence-conservative variants” of a nucleic acid are ones that have a different polynucleotide sequence but encode the same amino acid sequence.

In preferred embodiments, the homologous DISC1 nucleic acids of the invention (including, e.g., DISC1 analogs or variants) are ones which are associated with a neuropsychiatric disorder, such as schizophrenia, schizoaffective disorder, bipolar affective disorder, unipolar affective disorder and adolescent conduct disorder.

Examples of homologous DISC1 nucleic acids include allelic or species variants of the DISC1 nucleic acid sequence set forth in FIGS. 1A-C (SEQ ID NO:1). Preferred examples of such homologous sequences include the particular variants set forth in the Examples, infra.

Alternatively, DISC1 nucleic acid molecules of the invention may also be ones that hybridize to the complement of another DISC1 nucleic acid molecules (e.g., in a Southern blot assay) under any of the hybridization conditions defined in this specification. In a particular embodiment, a DISC1 nucleic acid molecule of the invention comprises a nucleotide sequence which hybridizes to the complement of a DISC1 nucleic acid sequence, such as the sequence set forth in FIGS. 1A-C (SEQ ID NO:1) or a portion thereof, under highly stringent hybridization conditions that comprise 50% formamide and 5× or 6×SSC. In other embodiments, the nucleic acid molecule of the invention hybridizes to a complement of a DISC1 nucleic acid sequence (e.g., the sequence set forth in FIGS. 1A-C and in SEQ ID NO:1, or a portion thereof) under moderately stringent hybridization conditions (e.g., 40% formamide with 5× or 6×SSC), or under low stringency hybridization conditions (e.g., in 5×SSC, 0.1% SDS, 0.25% mile, no formamide, 30% formamide, 5×SSC or 0.5% SDS). Particularly preferred hybridization conditions comprise hybridization at 42° C. in a low stringency hybridization buffer (e.g., 30% formamide, 10 mM Tris pH 7.6, 2.5×Denhardt's solution, 5×SSC, 0.5% SDS and 1.5 mg/ml sonicated salmon sperm DNA) followed by washing (preferably twice) at 50° C. using a low stringency washing buffer (e.g., 0.5×SSC and 1% SDS).

In other embodiments, the DISC1 nucleic acid molecules of the invention comprise fragments of a full length DISC1 sequence. For example, in preferred embodiments such DISC1 nucleic acid fragments comprise a nucleotide sequence that corresponds to a sequence of at least 10 nucleotides, preferably at least 15 nucleotides, more preferably at least 20 nucleotides and still more preferably at least 25 nucleotides of the full length DISC1 nucleotide sequence. In specific embodiments, the fragments correspond to a portion (e.g., of at least 10, 15, 20 or 25 nucleotides) of the DISC1 sequence set forth in FIGS. 1A-C (SEQ ID NO:1) or of another nucleotide sequence encoding the polypeptide sequence set forth in FIG. 2 (SEQ ID NO:2). In another embodiment, the fragments correspond to a portion (e.g., of at least 10, 15, 20, or 25 nucleotides) of a variant DISC1 sequence, such as a DISC1 sequence having a polymorphism. For example, in preferred embodiments such fragments may comprise one or more of the polymorphisms described in the Examples, infra.

In other preferred embodiments, the DISC1 nucleic acid fragments of the invention comprise sequence of at least 10, preferably at least 15, more preferably at least 20 and still more preferably at least 25 nucleotides that are complementary to and/or hybridize to a full length DISC1 nucleic acid sequence (e.g., the sequence set forth in FIGS. 1A-C and in SEQ ID NO:1) or to a portion thereof. In preferred embodiments, the nucleic acid fragments are complementary to a variant DISC1 nucleic acid sequence (for example, any of the variant sequences described in the Examples, infra) and/or specifically hybridize to such a variant DISC1 nucleic acid sequence. More preferably, the nucleic acid fragments of the invention are fragments which specifically hybridize to a variant DISC1 nucleic acid sequence and do not hybiridze to a regular or wild-type DISC1 sequence under the same, defined conditions.

Suitable hybridization conditions for such oligonucleotides are described supra, and include washing in 6×SSC/0.05% sodium pyrophosphate. Because the melting temperature of oligonucleotides will generally depend on the length of the oligonucleotide sequence, suitable hybridization temperatures may be expected to vary depending upon the oligonucleotide molecule used. Exemplary temperatures include 37° C. (for 14-base oligonucleotides), 48° C. (for 17-base oligonucleotides), 55° C. (for 20-base oligonucleotides) and 60° C. (for 23 or 25-base oligoncueltodies).

The DISC1 nucleic acid molecules of the invention also include “chimeric” DISC1 nucleic acids. Such chimeric nucleic acid molecules are polynucleotides which comprise at least one DISC1 nucleic acid sequence, and also at least one non-DISC1 nucleic acid sequence. The DISC1 nucleic acid sequence contained in a chimeric DISC1 nucleic acid molecule may be any of the wild-type, variant or polymorphic full length DISC1 sequences described here above, or may be any portion thereof (for example, a portion encoding a particular domain or containing a particular polymorphism). The non-DISC1 nucleic acid sequence may be, for example, a regulatory sequence (e.g., a promoter sequence) that is derived from another, non-DISC1 gene and is not normally associated with a naturally occurring DISC1 gene. The non-DISC1 nucleic acid sequence may also be a coding sequence for another, non-DISC1 polypeptide such as a FLAG, a histidine tag, glutathione S-transferase (GST), hemaglutinin, β-galactosidase, Green Fluorescent Protein (GFP), thioreductase or an immunoglobulin protein. In preferred embodiments, a chimeric nucleic acid molecule of the invention encodes a DISC1 fusion polypeptide of the invention (described below).

### DISC2

The DISC2 gene, as described supra, is a gene on human chromosome 1 which overlaps with, but is transcribed in the opposite direction of the human DISC1 gene described above. Unlike DISC1, DISC2 nucleic acids are not believed to encode polypeptides but, rather, encode a structural RNA gene product.

In preferred embodiments, a DISC2 nucleic acid molecule of the invention comprises the nucleotide sequence set forth in FIGS. 3A-G (SEQ ID NO:3), or a portion or fragment thereof. In another preferred embodiment, a DISC2 nucleic acid molecule of the invention may be a splice variant of the DISC2 sequence set forth in FIGS. 3A-G (SEQ ID NO:3). For example, DISC2 transcripts of at least 9.5 kb, and of approximately 6, 3 and 2.5 kb have been detected in heart tissue (see, Millar et al., Human Molecular Genetics 2000, 9:1415-1425). Such transcripts are among the DISC2 nucleic acid molecules of the present invention and, unless otherwise stated, may be used in any of the compositions and methods of the invention which recite use of a DISC2 nucleic acid.

The DISC2 nucleic acids of the present invention also include nucleic acid molecules that comprise modified or variant DISC2 nucleic acid sequences, including DISC2 sequences that have on or more nucleic acid insertions, substitutions, deletions or truncations. Such variant DISC2 nucleic acids may be, for example, analogs or homologs of a DISC2 nucleic acid in the same or in different species of organism. In preferred embodiments, such sequences have at least 50%, preferably at least 75% and more preferably at least 90% sequence identity to a DISC2 nucleotide sequence, such as the one set forth in FIGS. 3A-G (SEQ ID NO:3). The meanings of the terms “homologous” and “sequence identity” are as defined, supra, for DISC1 nucleic acid sequences of the invention.

In preferred embodiments, the modified or variant DISC2 nucleotide sequences are ones which are associated with a neuropsychiatric disorder such as schizophrenia, schizoaffective disorder, bipolar affective disorder, unipolar disorder and adolescent conduct disorder. Variant DISC1 nucleotides are described in the Examples, infra. Although specific DISC2 polymorphisms are not explicitly identified herein, the similarities between DISC1 and DISC2 enables a skilled artisan to practice the invention with respect to DISC2 based on the specific information provided for DISC1.

Alternatively, DISC2 nucleic acid molecules of the invention may also be ones that hybridize to the complement of another DISC2 nucleic acid molecules (e.g., in a Southern blot assay) under any of the hybridization conditions defined in this specification. In a particular embodiment, a DISC2 nucleic acid molecule of the invention comprises a nucleotide sequence which hybridizes to the complement of a DISC2 nucleic acid sequence, such as the sequence set forth in FIGS. 3A-G (SEQ ID NO:3) or a portion thereof, under highly stringent hybridization conditions that comprise 50% formamide and 5× or 6×SSC. In other embodiments, the nucleic acid molecule of the invention hybridizes to a complement of a DISC2 nucleic acid sequence (e.g., the sequence set forth in FIGS. 3A-G and in SEQ ID NO:3, or a portion thereof) under moderately stringent hybridization conditions (e.g., 40% formamide with 5× or 6×SSC), or under low stringency hybridization conditions (e.g., in 5×SSC, 0.1% SDS, 0.25% mile, no formamide, 30% formamid, 5×SSC or 0.5% SDS). Particularly preferred hybridization conditions comprise hybridization at 42° C. in a low stringency hybridization buffer (e.g., 30% formamide, 10 mM Tris pH 7.6, 2.5×Denhardt's solution, 5×SSC, 0.5% SDS and 1.5 mg/ml sonicated salmon sperm DNA) followed by washing (preferably twice) at 50° C. using a low stringency washing buffer (e.g. 0.5×SSC and 1% SDS).

In other embodiments, the DISC2 nucleic acid molecules of the invention comprise fragments of a full length DISC2 sequence. For example, in preferred embodiments such OSCAR nucleic acid fragments comprise a nucleotide sequence that corresponds to a sequence of at least 10 nucleotides, preferably at least 15 nucleotides, more preferably at least 20 nucleotides and still more preferably at least 25 nucleotides of the full length DISC2 nucleotide sequence. In specific embodiments, the fragments correspond to a portion (e.g., of at least 10, 15, 20 or 25 nucleotides) of the DISC2 sequence set forth in FIGS. 3A-G (SEQ ID NO:3). In another embodiment, the fragments correspond to a portion (e.g., of at least 10, 15, 20, or 25 nucleotides) of a variant DISC2 sequence, such as a DISC2 sequence having a polymorphism. Variant DISC1 nucleotide fragments are described in the Examples, infra. Again, although specific DISC2 fragments containing polymorphisms are not explicitly identified herein, the similarities between DISC1 and DISC2 enables a skilled artisan to practice the invention with respect to DISC2 based on the specific information provided for DISC1.

In other preferred embodiments, the DISC2 nucleic acid fragments of the invention comprise sequence of at least 10, preferably at least 15, more preferably at least 20 and still more preferably at least 25 nucleotides that are complementary to and/or hybridize to a full length DISC2 nucleic acid sequence (e.g., the sequence set forth in FIGS. 3A-G and in SEQ ID NO:3) or to a portion thereof. In preferred embodiments, the nucleic acid fragments are complementary to a variant DISC2 nucleic acid sequence (for example, any of the variant sequences described in the Examples, infra) and/or specifically hybridize to such a variant DISC2 nucleic acid sequence. More preferably, the nucleic acid fragments of the invention are fragments which specifically hybridize to a variant DISC2 nucleic acid sequence and do not hybridize to a regular or wild-type DISC2 sequence under the same, defined conditions.

Suitable hybridization conditions for such oligonucleotides are described supra, and include washing in 6×SSC/0.05% sodium pyrophosphate. Because the melting temperature of oligonucleotides will generally depend on the length of the oligonucleotide sequence, suitable hybridization temperatures may be expected to vary depending upon the oligonucleotide molecule used. Exemplary temperatures include 37° C. (for 14-base oligonucleotides), 48° C. (for 17-base oligonucleotides), 55° C. (for 20-base oligonucleotides) and 60° C. (for 23 or 25-base oligoncueltodies).

The DISC2 nucleic acid molecules of the invention also include “chimeric” DISC2 nucleic acids. Such chimeric nucleic acid molecules are polynucleotides which comprise at least one DISC2 nucleic acid sequence, and also at least one non-DISC2 nucleic acid sequence. The DISC2 nucleic acid sequence contained in a chimeric DISC2 nucleic acid molecule may be any of the wild-type, variant or polymorphic full length DISC2 sequences described here above, or may be any portion thereof (for example a portion encoding a particular domain or containing a particular polymorphism). The non-DISC2 nucleic acid sequence may be, for example, a regulatory sequence (e.g., a promoter sequence) that is derived from another, non-DISC2 gene and is not normally associated with a naturally occurring DISC2 gene. The non-DISC2 nucleic acid sequence may also be a coding sequence for another, non-DISC2 polypeptide such as a FLAG, a histidine tag, glutathione S-transferase (GST), hemaglutinin, β-galactosidase, Green Fluorescent Protein (GFP), thioreductase or an immunoglobulin protein.

Nucleic acid molecules comprising the above described DISC1 and DISC2 fragments are useful, for example, as oligonucleotide probes and primers (e.g., PCR primers) to detect and amplify other nucleic acid molecules encoding a DISC1 polypeptide or DISC2 RNA, including genes that encode variant DISC1 polypeptides or DISC2 RNA such as analogs, homologs and variants. Oligonucleotide fragments of the invention may also be used, e.g., as antisense nucleic acids, triple helix forming oligonucleotides or as ribozymes; e.g., to modulate levels of DISC1 or DISC2 gene expression or transcription in cells.

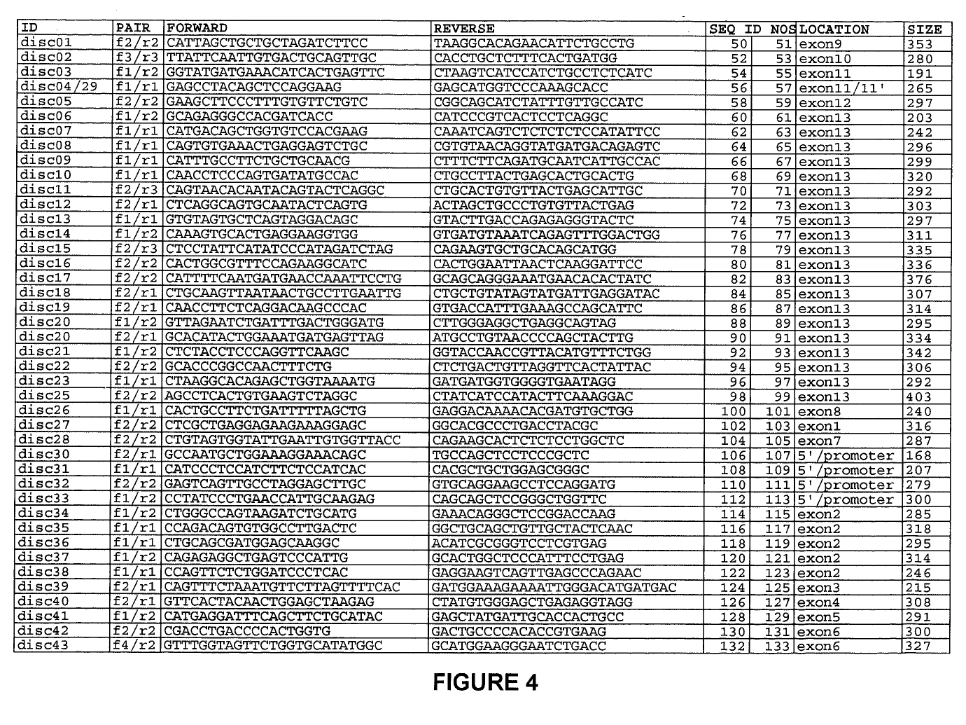

For example, FIG. 4 describes several specific nucleic acids, comprising the nucleotide sequences set forth in SEQ ID NOS:44-127, that may be used to amplify regions of a DISC1 or DISC2 gene or genomic sequence as described in the Examples. In particular, these sequences are used in the Examples to amplify particular segments of the DISC1 and DISC2 genomic sequence set forth in SEQ ID NOS:1 and 3, and identify nucleic acid mutations or polymorphisms (including single nucleotide polymorphisms) which correlate with and are therefore associated with a neuropsychiatric disorder. The nucleic acids of the present invention therefore include ones which comprise any of the nucleotide sequences set forth in FIG. 4, and in SEQ ID NOS:44-127.

The “primers” and “probes” of the invention are nucleic acid sequence which can be used for amplifying and/or identifying a DISC1 or DISC2 gene sequence. Primers can be used alone in a detection method, or a primer can be used together with at least one other primer or probe in a detection method. Primers can also be used to amplify at least a portion of a nucleic acid. Probes of the invention refer to nucleic acids which hybridize to the region of interest and which are not further extended. For example, a probe is a nucleic acid which specifically hybridizes to a polymorphic region of a DISC1 or DISC2 gene, and which by hybridization or absence of hybridization to the DNA of a subject will be indicative of the identity of the allelic variant of the polymorphic region of the DISC1 or DISC2 gene. Preferred probes include nucleic acid sequences comprising all or part of any of the nucleic acid sequences set forth in TABLE 5B (SEQ ID NOS:33-43).

Numerous procedures for determining the nucleotide sequence of a nucleic acid molecule, or for determining the presence of mutations in nucleic acid molecules include a nucleic acid amplification step, which can be carried out by, e.g., the polymerase chain reaction (PCR). Accordingly, in one embodiment, the invention provides primers for amplifying portions of a DISC1 or DISC2 gene, such as portions of exons and/or portions of introns. In a preferred embodiment, the exons and/or sequences adjacent to the exons of the human DISC1 or DISC2 gene will be amplified to, e.g., detect which allelic variant of a polymorphic region is present in the DISC1 or DISC2 gene of a subject. Preferred primers comprise a nucleotide sequence complementary a specific allelic variant of a DISC1 or DISC2 polymorphic region and of sufficient length to selectively hybridize with a DISC1 or DISC2 gene. In a preferred embodiment, the primer, e.g., a substantially purified oligonucleotide, comprises a region having a nucleotide sequence which hybridizes under stringent conditions to about 6, 8, 10, or 12, preferably 25, 30, 40, 50, or 75 consecutive nucleotides of a DISC1 or DISC2 gene. In an even more preferred embodiment, the primer is capable of hybridizing to a DISC1 or DISC2 nucleotide sequence and has a nucleotide sequence of any sequence set forth in any of SEQ ID NOS:33-127, complements thereof, allelic variants thereof, or complements of allelic variants thereof. For example, primers comprising a nucleotide sequence of at least about 15 consecutive nucleotides, at least about 25 nucleotides or having from about 15 to about 20 nucleotides set forth in any of SEQ ID NOS:33-127, or complement thereof are provided by the invention. Primers having a sequence of more than about 25 nucleotides are also within the scope of the invention. Preferred primers of the invention are primers that can be used in PCR for amplifying each of the exons of a DISC1 or DISC2 gene.

Primers can be complementary to nucleotide sequences located close to each other or further apart, depending on the use of the amplified DNA. For example, primers can be chosen such that they amplify DNA fragments of at least about 10 nucleotides or as much as several kilobases. Preferably, the primers of the invention will hybridize selectively to nucleotide sequences located about 150 to about 350 nucleotides apart.

For amplifying at least a portion of a nucleic acid, a forward primer (i.e., 5′ primer) and a reverse primer (i e., 3′ primer) will preferably be used. Forward and reverse primers hybridize to complementary strands of a double stranded nucleic acid, such that upon extension from each primer, a double stranded nucleic acid is amplified. Forward and reverse primers are shown in FIG. 4 (SEQ ID NOS:44-127).

DISC1 or DISC2 nucleic acid molecules of the invention, whether genomic DNA, cDNA, mRNA or otherwise, can be isolated from any source including, for example, cDNA or genomic libraries. Preferably, the cDNA library is a library generated from cells, tissue or organ, such as brain, which expresses a DISC1 or DISC2 gene of the invention. Methods for obtaining particular genes (i.e., DISC1 or DISC2 genes and nucleic acids) from such libraries are well known in the art, as described above (see, e.g., Sambrook et al., 1989, supra).

The DNA may be obtained by standard procedures known in the art from cloned DNA (for example, from a DNA “library”), and preferably is obtained from a cDNA library prepared from cells or tissue with high level expression of the gene or its gene product (for example, from brain cells or tissue). In one embodiment, the DNA may be obtained from a “subtraction” library to enrich the library for cDNAs of genes specifically expressed by a particular cell type or under certain conditions. In still other embodiments, a library may be prepared by chemical synthesis, by cDNA cloning, or by the cloning of genomic DNA or fragments thereof purified from the desired cell (see, for example, Sambrook et al., 1989, supra; Glover, D. M. edl, 1985. DNA Cloning: A Practical Approach, MRL. Press, Ltd., Oxford, U.K. Vols. I and II).

Clones derived from genomic DNA may contain regulatory and intron DNA regions in addition to coding regions. Clones derived from cDNA generally will not contain intron sequences. Whatever the source, the gene is preferably molecularly cloned into a suitable vector for propagation of the gene. Identification of the specific DNA fragment containing the desired DISC1 or DISC2 gene may be accomplished in a number of ways. For example, a portion of a DISC1 or DISC2 gene exemplified infra can be purified and labeled to prepare a labeled probe (Benton & Davis, Science 1977, 196:180; Grunstein & Hogness, Proc. Natl. Acad. Sci. U.S.A. 1975, 72:3961). Those DNA fragments with substantial homology to the probe, such as an allelic variant from another individual, will hybridize thereto. In a specific embodiment, highest stringency hybridization conditions are used to identify a homologous DISC1 or DISC2 gene.

Further selection can be carried out on the basis of properties of the DISC1 or DISC2 gene product; such as if the gene encodes a protein product having the isoelectric electrophoretic, amino acid composition, partial or complete amino acid sequence, antibody binding activity or ligand binding profile of a DISC1 polypeptide as disclosed herein. Thus, the presence of the gene may be detected by assays based on the physical, chemical, immunological or functional properties of its expressed product.

Other DNA sequences which encode substantially the same amino acid sequence as a DISC1 gene may be used in the practice of the present invention. These include, but are not limited to allelic variants, species variants, sequence conservative variants, and functional variants. In particular, the nucleic acid sequences of the invention include both “function-conservative variants” and “sequence-conservative variants”. Nucleic acid substitutions may be made, for example, to alter the amino acid residue encoded by a particular codon, and thereby substitute an amino acid sequence in a DISC1 polypeptide for one with a particularly preferred property.

### Polymorphisms in DISC1 and DISC2

The present invention also provides, in preferred embodiments, variant DISC1 and DISC2 nucleic acids including variants which comprise one or more single nucleotide polymorphisms (SNPs). As an example, and not by way of limitation, TABLE 5, infra, discloses several single nucleotide polymorphisms (SNPs) of the DISC1 genomic sequences set forth in TABLE 3. TABLE 6A discloses similar SNPs of the DISC1 cDNA sequence set forth in SEQ ID NO:1. In addition, the Examples, infra, demonstrate that these SNPs are ones which correlate with neuropsychiatric disorders. Accordingly, DISC1 and DISC2 nucleic acid molecules which comprise one or more of these SNPs are particularly preferred embodiments of DISC1 and DISC2 nucleic acids of the present invention.

The polymorphic sequences of the invention can advantageously be used as primers to amplify an allelic variant of a DISC1 and/or DISC2 gene, i.e., nucleic acids which are capable of selectively hybridizing to an allelic variant of a polymorphic region of a DISC1 and/or DISC2 gene. Thus, such primers ;an be specific for a DISC1 and/or DISC2 gene sequence, so long as they have a nucleotide sequence which is capable of hybridizing to a DISC1 and/or DISC2 gene. Preferred primers are capable of specifically hybridizing to any of the allelic variants listed in TABLE 5. Such primers can be used, e.g., in sequence specific oligonucleotide priming as described further herein.

The DISC1 and DISC2 nucleic acids of the invention can also be used as probes, e.g., in therapeutic and diagnostic assays. For instance, the present invention provides a probe comprising a substantially purified oligonucleotide, which oligonucleotide comprises a region having a nucleotide sequence that is capable of hybridizing specifically to a region of a DISC1 and DISC2 gene which is polymorphic (see TABLE 5, infra). In an even more preferred embodiment of the invention, the probes are capable of hybridizing specifically to an allelic variant of a DISC1 and/or DISC2 gene, and comprise all or part of the sequences set forth in TABLE 5B (SEQ ID NOS:33-43). Such probes can then be used to specifically detect which allelic variant of a polymorphic region of a DISC1 and/or DISC2 gene is present in a subject. The polymorphic region can be located in the promoter, exon, or intron sequences of a DISC1 or DISC2 gene.

For example, preferred probes of the invention are those probes listed in TABLE 5B, wherein the bold nucleotides represent the location of the nucleotide polymorphism. For each probe listed in TABLE 5B, the complement of that probe is also included in the table as a preferred probe of the invention. Particularly preferred probes of the invention have a number of nucleotides sufficient to allow specific hybridization to the target nucleotide sequence. Where the target nucleotide sequence is present in a large fragment of DNA, such as a genomic DNA fragment of several tens or hundreds of kilobases, the size of the probe may have to be longer to provide sufficiently specific hybridization, as compared to a probe which is used to detect a target sequence which is present in a shorter fragment of DNA. For example, in some diagnostic methods, a portion of a DISC1 and/or DISC2 gene may first be amplified and thus isolated from the rest of the chromosomal DNA and then hybridized to a probe. In such a situation, a shorter probe will likely provide sufficient specificity of hybridization. For example, a probe having a nucleotide sequence of about 10 nucleotides may be sufficient.

In preferred embodiments, the probe or primer further comprises a label attached thereto, which, e.g., is capable of being detected, e.g. the label group is selected from amongst radioisotopes, fluorescent compounds, enzymes, and enzyme co-factors.

In another preferred embodiment of the invention, the isolated nucleic acid, which is used, e.g., as a probe or a primer, is modified, such as to become more stable. Exemplary nucleic acid molecules which are modified include phosphoramidate, phosphothioate and methylphosphonate analogs of DNA (see also U.S. Pat. Nos. 5,176,996; 5,264,564; and 5,256,775).

The nucleic acids of the invention can also be modified at the base moiety, sugar moiety, or phosphate backbone, for example, to improve stability of the molecule. The nucleic acids, e.g., probes or primers, may include other appended groups such as peptides (e.g., for targeting host cell receptors in vivo), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. WO88/09810, published Dec. 15, 1988), hybridization-triggered cleavage agents. (See, e.g., Krol et al, 1988, BioTechniques 6:958-976) or intercalating agents. (See, e.g., Zon, 1988, Pharm. Res. 5:539-549). To this end, the nucleic acid of the invention may be conjugated to another molecule, e.g., a peptide, hybridization triggered cross-linking agent, transport agent, hybridization-triggered cleavage agent, etc.

The isolated nucleic acid comprising a DISC1 and DISC2 intronic sequence may comprise at least one modified base moiety which is selected from the group including but not limited to 5-fluorouracil, 5-bromouracil, 5-chlorouracil, 5-iodouracil, hypoxanthine, xantine, 4-acetylcytidine, 5-(carboxyhydroxymethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethyl guanine, 2-methyladenine, 2-methylguanine, 3-methylcytidine, 5-methylcytidine, N6-adenine, 7-methylguanine,5-methylaminomethyluracil,5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytidine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

The isolated nucleic acid may also comprise at least one modified sugar moiety selected from the group including but not limited to arabinose, 2-fluoroarabinose, xylulose, and hexose.

In yet another embodiment, the nucleic acid comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet a further embodiment, the nucleic acid is an α-anomeric oligonucleotide. An α-anomeric oligonucleotide forms specific double-stranded hybrids with complementary RNA in which, contrary to the usual β-units, the strands run parallel to each other (Gautier et al., 1987, Nucl. Acids Res. 15:6625-6641). The oligonucleotide is a 2′-0-methylribonucleotide (Inoue et al., 1987, Nucl. Acids Res. 15:6131-6148), or a chimeric RNA-DNA analogue (Inoue et al., 1987, FEBS Lett. 215:327-330).

Any nucleic acid fragment of the invention can be prepared according to methods well known in the art and described, e.g., in Sambrook, J. Fritsch, E. F., and Maniatis, T. (1989) Molecular Cloning: A Laboratory Manual, Cold Spring Harbor Laboratory Press, Cold Spring Harbor, N.Y. For example, discrete fragments of the DNA can be prepared and cloned using restriction enzymes. Alternatively, discrete fragments can be prepared using the Polymerase Chain Reaction (PCR) using primers having an appropriate sequence.

Oligonucleotides of the invention may be synthesized by standard methods known in the art, e.g. by use of an automated DNA synthesizer (such as are commercially available from Biosearch, Applied Biosystems, etc.). As examples, phosphorothioate oligonucleotides may be synthesized by the method of Stein et al. 1988, Nucl. Acids Res. 16:3209), methylphosphonate oligonucleotides can be prepared by use of controlled pore glass polymer supports (Sarin et al., 1988, Proc. Natl. Acad. Sci. U.S.A. 85:7448-7451), or other means know in the art.

The invention also provides other variants of DISC1 and DISC2 nucleic acids, including nucleic acids having variant microsatellite repeats. A “microsatellite repeat” or “microsatellite”, as the term is used herein, refers to a short sequence of repeating nucleotides within a nucleic acid. Typically, a microsatellite repeat comprises a repeating sequence of two (i.e., a dinucleotide repeat), three (i.e., a trinucleotide repeat), four (i.e., a tetranucleotide repeat) or five (i.e., a pentanucleotide repeat) nucleotides. Microsatellites of the invention therefore have the general formula (N₁, N₂, . . . Nᵢ)ₙ, wherein N represents a nucleic acid residue (e.g., adenine, thymine, cytosine or guanine), i represents the number of the last nucleotide in the microsatellite, and n represents the number of times the motif is repeated in the microsatellite locus. In one embodiment the number of nucleotides in a microsatellite motif (i) is about six, preferably between two and five, and more preferably two, three or four. The total number of repeats (n) in a microsatellite repeat may be, e.g., from one to about 60, preferably from 4 to 40, and more preferably from 10 to 30 when i=2; is preferably between about 4-25, and more preferably between about 6-22 when i=3; and is preferably between about 4-15, and more preferably between about 5-10 when i=4. A DISC1 and DISC2 nucleic acid of the invention may comprise any microsatellite repeat of the above general formula. However, the following motifs are particularly preferred: CA, TC, and, AATTG; as well as all complements and permutations of such motifs (for example, TG, GA, and CAATT. These variant DISC1 and DISC2 nucleic acids are also considered part of the present invention.

Accordingly, the nucleic acid molecules of the present invention include DISC1 and DISC2 nucleic acid molecules having one or more of the polymorphisms described in TABLES 5and 6 (SEQ ID NOS: 33-43). In preferred embodiments, the nucleic acid molecules of the invention include specific DISC1 and DISC2 allelic variants, which differ from the reference or wild-type DISC1 and DISC2 nucleic acid molecules described supra (i.e., nucleic acid molecules having the nucleotide sequence set forth in SEQ ID NOS 1 and 3, and in TABLE 3).

The genes encoding DISC1 and/or DISC2 derivatives and analogs of the invention can be produced by various methods known in the art. The manipulations which result in their production can occur at the gene or protein level. For example, the cloned DISC1/DISC2 gene sequence can be modified by any of numerous strategies known in the art (see, e.g., Sambrook et al., 1989, supra). The sequence can be cleaved at appropriate sites with restriction endonuclease(s), followed by further enzymatic modification if desired, isolated, and ligated in vitro. In the production of the gene encoding a derivative or analog DISC1 and/or DISC2, care should be taken to ensure that the modified gene remains within the same translational reading frame as the DISC1/DISC2 gene, uninterrupted by translational stop signals, in the gene region where the desired activity is encoded.

Additionally, the DISC1 and DISC2-encoding nucleic acid sequences can be mutated in vitro or in vivo, to create and/or destroy translation, initiation and/or termination sequences, or to create variations in coding regions and/or form new restriction endonuclease sites or destroy preexisting ones, to facilitate further in vitro modification. Modifications can also be made to introduce restriction sites and facilitate cloning the DISC1 /DISC2 genes into an expression vector. An technique for mutagenesis known in the art can be used, including but not limited to, in vitro site-directed mutagenesis (Hutchinson et al., J. Biol. Chem. 1978, 253:6551; Zoller & Smith, DNA 1984, 3:479-488; Oliphant et al., Gene 1986, 44:177; Hutchinson et al., Proc. Natl. Acad. Sci. U.S.A. 1986, 83:710), use of TAB″ linkers (Pharmacia), etc. PCR techniques are preferred for site directed mutagenesis (see, Higuchi, 1989, “Using PCR to Engineer DNA” in PCR Technology: Principles and Applications for DNA Amplification, H. Erlich, ed., Stockton Press, Chapter 6, pp. 61-70).

The identified and isolated gene can then be inserted into an appropriate cloning vector. A large number of vector-host systems known in the art may be used. Possible cloning vectors include, but are not limited to, plasmids or modified viruses. The vector system must, however, by compatible with the host cell used. Examples of vectors include, but are not limited to, E. coli, bacteriophages such as lambda derivatives, or plasmids such as pBR322 derivatives or pUC plasmid derivatives, e.g., pGEX vectors, pmal-c, pFLAG, pKK plasmids (Clonetech), pET plasmids (Novagen, Inc., Madison, Wis.), pRSET or pREP plasmids, pcDNA (Invitrogen, Carlsbad, Calif.), pMAL plasmids (New England Biolabs, Beverly, Mass.), etc. The insertion into a cloning vector can, for example, be accomplished by ligating the DNA fragment into a cloning vector which has complementary cohesive termini. However, if the complementary restriction sites used to fragment the DNA are not present in the cloning vector, the ends of the DNA molecules may be enzymatically modified. Alternatively, any site desired may be produced by ligating nucleotide sequences (i.e., “linkers”) onto the DNA termini. These ligated linkers may comprise specific chemically synthesized oligonucleotides encoding restriction endonuclease recognition sequences.

Recombinant molecules can be introduced into host cells via transformation, transfection, infection, electroporation, etc., so that many copies of the gene sequence are generated. Preferably, the cloned gene is contained on a shuttle vector plasmid, which provides for expansion in a cloning cell (for example, E. coli) and facile purification for subsequent insertion into an appropriate expression cell line, if such is desired. For example, a shuttle vector, which is a vector that can replicate in more than one type of organism, can be prepared for replication in both E coli and Saccharomyces cerevisiae by linking sequences from an E. coli plasmid with sequence from the yeast 2 m plasmid.

### Isolating DISC1 and DISC2 Nucleic Acids

DISC1 and DISC2 nucleic acid molecules of the invention, whether genomic DNA, cDNA or otherwise, can be isolated from any source. Methods for obtaining DISC1 and DISC2 gene are well known in the art, as described above (see, e.g., Sambrook et al., 1989, supra).

The DNA may be obtained by standard procedures known in the art from cloned DNA (for example, from a DNA “library”), and preferably is obtained from a cDNA library prepared from tissues with high level expression of the gene product. In other embodiments, a library may be prepared by chemical synthesis, by cDNA cloning, or by the cloning of genomic DNA or fragments thereof, purified from the desired cell (See, for example, Sambrook et al., 1989, supra; Glover, D. M. ed., 1985, DNA Cloning: A Practical Approach, MRL Press, Ltd., Oxford, U.K. Vols. I and II).

Clones derived from genomic DNA may contain regulatory and intron DNA region in addition to coding regions. Clones derived from cDNA generally will not contain intron sequences. Whatever the source, the gene should be molecularly cloned into a suitable vector for propagation of the gene. Identification of the specific DNA fragment containing the desired DISC1 or DISC2 gene may be accomplished in a number of ways. For example, a portion of an DISC1 or DISC2 gene exemplified infra can be purified and labeled to prepare a labeled probe (Benton & Davis, Science 1977, 196:180; Grunstein & Hogness, Proc. Natl. Acad. Sci. U.S.A. 1975, 72:3961). Those DNA fragments with substantial homology to the probe, such as an allelic variant from another individual, will hybridize. In a specific embodiment, highest stringency hybridization conditions are used to identify a homologous DISC1 or DISC2 gene.

Further selection can be carried out on the basis of the properties of the gene, e.g., if the gene encodes a protein product having the isoelectric, electrophoretic, amino acid composition, partial or complete amino acid sequence, antibody binding activity, or ligand binding profile of DISC1 or DISC2 protein as disclosed herein. Thus, the presence of the gene may be detected by assays based on the physical, chemical, immunological, or functional properties of its expressed product.

Other DNA sequences which encode substantially the same amino acid sequence as an OSCAR gene may be used in the practice of the present invention. These include but are not limited to allelic variants, species variants, sequence conservative variants, and functional variants. In particular, the nucleic acid sequences of the invention include both “function-conservative variants” and “sequence-conservative variants”. Function-conservative variants of a nucleic acid are those nucleic acids which encode a function-conservative variant of a polypeptide, as defined supra. “Sequence-conservative variants” of a nucleic acid are nucleic acids that have a different polynucleotide sequence but encode the same amino acid sequence.

Amino acid substitutions may also be introduced to substitute an amino acid with a particularly preferable property. For example, a Cys may be introduced a potential site for disulfide bridges with another Cys.

The genes encoding DISC1 or DISC2 derivatives and analogs of the invention can be produced by various methods known in the art. The manipulations which result in their production can occur at the gene or protein level. For example, the cloned OSCAR gene sequence can be modified by any of numerous strategies known in the art (Sambrook et al., 1989, supra). The sequence can be cleaved at appropriate sites with restriction endonuclease(s), followed by further enzymatic modification if desired, isolated, and ligated in vitro. In the production of the gene encoding a derivative or analog of DISC1 or DISC2, care should be taken to ensure that the modified gene remains within the same translational reading frame as the wild-type gene, uninterrupted by translational stop signals, in the gene region where the desired activity is encoded.

Additionally, the DISC1 or DISC2 nucleic acid sequence can be mutated in vitro or in vivo, to create and/or destroy translation, initiation, and/or termination sequences, or to create variations in coding regions and/or form new restriction endonuclease sites or destroy preexisting ones, to facilitate further in vitro modification. Modifications can also be made to introduce restriction sites and facilitate cloning the DISC1 or DISC2 gene into an expression vector. Any technique for mutagenesis known in the art can be used, including but not limited to, in vitro site-directed mutagenesis (Hutchinson, C., et al., J. Biol. Chem. 253:6551, 1978; Zoller and Smith, DNA 3:479-488, 1984; Oliphant et al., Gene 44:177, 1986; Hutchinson et al., Proc. Natl. Acad. Sci. U S.A. 83:710, 1986), use of TAB″ linkers (Pharmacia), etc. PCR techniques are preferred for site directed mutagenesis (see Higuchi, 1989, “Using PCR to Engineer DNA” in PCR Technology: Principles and Applications for DNA Amplification, H. Erlich, ed., Stockton Press, Chapter 6, pp. 61-70).

The identified and isolated gene can then be inserted into an appropriate cloning vector. A large number of vector-host systems known in the art may be used. Possible vectors include, but are not limited to, plasmids or modified viruses, but the vector system must be compatible with the host cell used. Examples of vectors include, but are not limited to, E. coli, bacteriophages such as lambda derivatives or plasmids such as pBR322 derivatives or pUC plasmid derivatives, e.g., pGEX vectors, pmal-c, pFLAG, pKK plasmids (Clonetech), pET plasmids (Novagen, Inc., Madison, Wis.), pRSET or pREP plasmids (Invitrogen, San Diego, Calif.), or pMAL plasmids (New England Biolabs, Beverly, Mass.), etc. The insertion into a cloning vector can, for example, be accomplished by ligating the DNA fragment into a cloning vector which has complementary cohesive termini. However, if the complementary restriction sites used to fragment the DNA are not present in the cloning vector, the ends of the DNA molecules may be enzymatically modified. Alternatively, any site desired may be produced by ligating nucleotide sequences (linkers) onto the DNA termini; these ligated linkers may comprise specific chemically synthesized oligonucleotides encoding restriction endonuclease recognition sequences.

Recombinant molecules can be introduced into host cells via transformation, transfection, infection, electroporation, etc., so that many copies of the gene sequence are generated. Preferably, the cloned gene is contained on a shuttle vector plasmid, which provides for expansion in a cloning cell, e.g., E. coli, and facile purification for subsequent insertion into an appropriate expression cell line, if such is desired. For example, a shuttle vector, which is a vector that can replicate in more than one type of organism, can be prepared for replication in both E coli and Saccharomyces cerevisiae by linking sequences from an E. coli plasmid with sequences form the yeast 2 m plasmid.

### 5.3 DISC1 and DISC2 Gene Products

Gene products of the DISC1 and DISC2 genes are described above. As explained above, the DISC2 gene is not predicted to encode a polypeptide or protein. Rather, DISC2 is thought to be a non-protein coding gene for a structural RNA. As such, the DISC2 gene products of the present invention are, themselves, DISC2 nucleic acids. These DISC2 gene products may include, therefore, all of the variations and modifications described supra for DISC2 nucleic acids of the invention.

The DISC1 gene is predicted to encode a polypeptide having the amino acid sequence shown in FIG. 2 and set forth in SEQ ID NO:2. This DISC1 polypeptide may be divided into at least two regions of distinct secondary structure. The first region, which is referred to as the N-terminal region, comprises approximately amino acid residues 1-347 of SEQ ID NO:2 and is predicted to comprise at least one globular domain. The second region of secondary structure is also referred to as the C-terminal domain and comprises approximately amino acid residues 348-854 of SEQ ID NO:2. The C-terminal region comprises at least three α-helix regions which are indicated by bold underlining in FIG. 2. The α-helix regions correspond to approximately amino acid residues 367-394, 452-500 and 602-630 of SEQ ID NO:2. It is understood that the α-helices contained in the C-terminal region may interact with each other to form coiled coil structures.

A splice variant of the DISC1 gene product is also known to exist. This splice variant of the DISC1 gene product is predicted to encode a polypeptide having the amino acid sequence shown in FIG. 2 (SEQ ID NO:2). However, the sequence corresponding to the amino acid residues indicated by light underlining in FIG. 2 (i.e., amino acid residues 748-768 of SEQ ID NO:2) is omitted from this splice variant of the DISC1 gene product.

In one specific embodiment, a DISC1 polypeptide is a polypeptide from a human cell or tissue and, more preferably, from a human brain cell or tissue. For example, a human DISC1 polypeptide of the invention may comprise the amino acid sequence set forth in SEQ ID NO:2. Likewise, a DISC2 RNA is RNA from a human cell or tissue and, more preferably, from a human brain cell or tissue, which sequence corresponds to a sequence transcribed from a wild-type or variant DISC2 gene.

In other embodiments, DISC1 and DISC2 gene products also include fragments of a full length DISC1 protein or DISC1 RNA. For example, polypeptides which may be used in the compositions and methods of the present invention include polypeptides comprising an epitope of a full length DISC1 polypeptide, such as epitopes of the full length DISC1 polypeptide shown in FIG. 1B (SEQ ID NO:2) or a splice variant thereof (for example, the splice variant described above). An epitope of a DISC1 polypeptide represents a site on the polypeptide against which an antibody may be produced and to which the antibody binds. Thus, polypeptides comprising the amino acid sequence of a DISC1 epitope are useful for making antibodies to a DISC1 protein. Preferably, an epitope comprises a sequence of at least 5, more preferably at least 10, 15, 20, 25 or 50 amino acid residues in length. Thus, DISC1 polypeptides of the invention that comprise epitopes of a DISC1 protein preferably contain at least 5, at least 10, at least 15, at least 20, at least 25 or at least 50 amino acid residues of a DISC1 protein sequence. For example, in certain preferred embodiments wherein the epitope is an epitope of the DISC1 polypeptide set forth in FIG. 2 (SEQ ID NO:2), a DISC1 polypeptide of the invention may comprise an amino acid sequence corresponding to a sequence of at least 5, at least 10, at least 15, at least 20, at least 25 or at least 50 amino acid residues of the sequence set forth in FIG. 2 (SEQ ID NO:2).

The DISC1 polypeptides or DISC2 RNA sequences of the invention also include analogs and derivatives of the full length DISC1 polypeptides set forth in FIG. 2 (SEQ ID NO:2) or the full length DISC2 RNA sequence. Analogs and derivatives of the DISC1 polypeptides or DISC2 RNA sequences of the invention have the same or homologous characteristics of DISC1 polypeptides or DISC2 RNA sequences set forth above.

A DISC1 chimeric fusion polypeptide can be prepared in which the DISC1 portion of the fusion protein has one or more characteristics of the DISC1 polypeptide. Such fusion polypeptides therefore represent embodiments of the DISC1 polypeptides of the present invention. Exemplary DISC1 fusion polypeptides include ones which comprise a full length, derivative or truncated DISC1 amino acid sequence, as well as fusions which comprise a fragment of an OSCAR polypeptide sequence (e g., a fragment corresponding to an epitope or to one or more domains or regions). Such fusion polypeptides may also comprise the amino acid sequence of a marker polypeptide; for example FLAG, a histidine tag, glutathione S-transferase (GST) or hemaglutinin. In other embodiments, a DISC1 polypeptide may be expressed with a bacterial protein such as β-galactosidase. Additionally, DISC1 fusion polypeptides may comprise amino acid sequences that increase solubility of the polypeptide, such as a thioreductase amino acid sequence or the sequence of one or more immunoglobulin proteins (e.g., IgG1 or IgG2).

DISC1 analogs or variants can also be made by altering the wild-type DISC1 polypeptide sequence, e.g., by introducing one or more amino acid residue substitutions, additions or deletions. In one embodiment, such altered polypeptides are functionally similar molecules (i.e., molecules that perform one or more DISC1 functions or have one or more DISC1 bioactivities). Thus, in a specific embodiment, an analog of a DISC1 polypeptide is a function-conservative variant, as the term is defined supra.

In a preferred embodiment, an analog of a DISC1 polypeptide is associated with a neuropsychiatric disorder, such as schizophrenia, schizoaffective disorder, bipolar affective disorder, unipolar affective disorder and adolescent conduct disorder. For example, the Examples infra describe various mutations to the DISC1 gene which encode an analog DISC1 gene product. Such analog DISC1 gene products represent exemplary, specific embodiments of the analog DISC1 gene products of the present invention.

Amino acid residues, other than the ones that are specifically identified herein as being conserved, may differ among variants of a protein or polypeptide. Accordingly, the percentage of protein or amino acid sequence similarity between any two DISC1 polypeptides of similar function may vary. Typically, the percentage of protein or amino acid sequence similarity between different DISC1 polypeptide analogs or variants may be from 70% to 99%, as determined according to an alignment scheme such as the Cluster Method and/or the MEGALIGN algorithm. Analogs and variants of a DISC1 polypeptide therefore also include polypeptide that have at least 50%, preferably at least 75%, more preferably at least 85% and still more preferably at least 90% amino acid sequence identity as determined, e.g., by BLAST or FASTA algorithms. In one embodiment, such analogs and variants of a DISC1 polypeptide are function-conservative variants which have the same or similar properties, functions or bioactivities as the native polypeptide to which they are compared. In another, preferred embodiment, such analogs and variants of a DISC1 polypeptide are ones which are associated with a neuropsychiatric disorder, such as schizophrenia, schizoaffective disorder, bipolar affective disorder, unipolar affective disorder and adolescent conduct disorder. It is further noted that the analogs of the DISC1 polypeptides of the present invention include, not only homologs and variants of the full length DISC1 polypeptides of the present invention (e.g., variants of a DISC1 polypeptide comprising the sequence set forth in FIG. 2 and in SEQ ID NO:2), but also include variants of modified DISC1 polypeptides (e.g., truncations and deletions) and of fragments (e.g., corresponding to particular domains, regions or epitopes) of a full length DISC1 polypeptide.

In yet other embodiment, an analog of a DISC1 polypeptide is an allelic variant or mutant of a DISC1 polypeptide. The term allelic variant and mutant, when used to describe a polypeptide, refers to a polypeptide encoded by an allelic variant or mutant gene. Thus, the allelic variant and mutant DISC1 polypeptides of the invention are polypeptides encoded by allelic variants of mutants of the DISC1 nucleic acid molecules of the present invention.

In yet other embodiments, an analog of a DISC1 polypeptide is a substantially homologous polypeptide from the same species (e.g., an allelic variant) or from another species (e.g., an orthologous polypeptide); preferably from another mammalian species such as mouse, rat, rabbit, hamster, guinea pig, primate (e.g., monkey or human), cats, dogs, sheep, goats, pigs, horses, cows, etc. However, DISC1 homologous of the invention may be from any species, including chickens, Xenopus, yeast (e.g., Saccharomyces cerevisiae), and bacteria (e.g., E. coli) to name a few.

In a specific embodiment, two polypeptide sequence are “substantially homologous” or “substantially similar” when the polypeptides are at least 35-40% similar as determined by one of the algorithms disclosed herein, preferably at least about 60% similar, and more preferably at least about 90 or 95% similar in one or more highly conserved domains or, for allelic variants, across the entire amino acid sequence.

In other embodiments, variants of a DISC1 polypeptide (including analogs, orthologs and homologs) are polypeptides encoded by nucleic acid molecules that hybridize to the complement of a nucleic acid molecule encoding a DISC1 polypeptide; e.g., in a Southern hybridization experiment under defined conditions. For example, in a particular embodiment analogs and/or homologs of a DISC1 polypeptide comprise amino acid sequences encoded by nucleic acid molecules that hybridize to a complement of a DISC1 nucleic acid sequence, such as the coding sequence of the DISC1 cDNA sequence set froth in FIGS. 1A-C (SEQ ID NO:1), under highly stringent hybridization conditions that comprise 50% formamide and 5× or 6×SSC. in other embodiments, the analogs and/or homologs of a DISC1 polypeptide may comprise amino acid sequences encoded by nucleic acid molecules that hybridize to a complement of a DISC1 nucleic acid sequence (e.g., the complement of the coding sequence of the DISC1 cDNA sequence set forth in FIGS. 1A-C and in SEQ ID NO:1) under moderately stringent hybridization conditions (i.e., 40% formamide with 5× or 6×SSC), or under low stringency conditions (e.g., in 5×SSC, 0.1% SDS, 0.25% milk, no formamide, 30% formamide, 5×SSC or 0.5% SDS).

In still other embodiments, variants (including analogs, homologs and orthologs) of a DISC1 polypeptide can also be identified by isolating variant DISC1 genes, e.g., by PCR using degenerate oligonucleotide primers designed on the basis of amino acid sequence of the DISC1 polypeptide (see, supra).

Derivatives of the DISC1 polypeptides of the invention further include, but are by no means limited to, phosphorylated DISC1, myristylated DISC1, methylated DISC1 and other DISC1 polypeptides that are chemically modified. DISC1 polypeptides of the invention may include labeled variants; form example, radio-labeled with iodine or phosphorous (see, e.g., EP 372707B) or other detectable molecule such as, but by no means limited to, biotin, a fluorescent dye (e.g., Cy5 or Cy3), a chelating group complexed with a metal ion, a chromophore or fluorophore, a gold colloid, a particle -such as a latex bead, or attached to a water soluble polymer.

Chemical modification of a biologically active component or components of DISC1 (or DISC2) nucleic acids or polypeptides may provide additional advantages under certain circumstances. See, for example, U.S. Pat. No. 4,179,337 issued Dec. 18, 1970 to Davis et al. Also, for a review see Abuchowski et al., in Enzymes as Drugs (J. S. Holcerberg and J. Roberts, eds. 1981), pp. 367-383. A review article describing protein modification and fusion proteins is found in Francis, Focus on Growth Factors 1992, 3:4-10, Mediscript: Mountview Court, Friem Barnet Lane, London N20, OLD, UK.

### Polymorphisms in the DISC1 polypeptide

The present invention provides isolated polymorphic DISC1 polypeptides, such as DISC1 polypeptides which are encoded by specific allelic variants of DISC1 genes, including those identified herein. Accordingly, preferred DISC1 polypeptides of the invention have an amino acid sequence which differs from SEQ ID NO:2. In one embodiment, the DISC1 polypeptides are isolated from, or otherwise substantially free of other cellular proteins. The term “substantially free of other cellular proteins” (also referred to herein as “contaminating proteins”) or “substantially pure or purified preparations” are defined as encompassing preparations of DISC1 polypeptides having less than about 20% (by dry weight) contaminating protein, and preferably having less than about 5% contaminating protein. It will be appreciated that functional forms of the subject polypeptides can be prepared, for the first time, as purified preparations by using a cloned gene as described herein.

Preferred DISC1 proteins of the invention have an amino acid sequence which is at least about 60%, 70%, 80%, 85%, 90%, or 95% identical or homologous to an amino acid sequence of SEQ ID NO:2. Even more preferred DISC1 proteins comprise an amino acid sequence which is at least about 97, 98, or 99% homologous or identical to an amino acid sequence of SEQ ID NO:2. Such proteins can be recombinant proteins, and can be, e.g., produced in vitro from nucleic acids comprising a specific allele of a DISC1 polymorphic region. For example, recombinant polypeptides preferred by the present invention can be encoded by a nucleic acid, which is at least 85% homologous and more preferably 90% homologous and most preferably 95% homologous with a nucleotide sequence set forth in SEQ ID NO:1, and comprises an allele of a polymorphic region that differs from that set forth in SEQ ID NO:1. Polypeptides which are encoded by a nucleic acid that is at least about 98-99% homologous with the sequence of SEQ ID NO:1, and comprise an allele of a polymorphic region that differs from that set forth in SEQ ID NO:1 are also within the scope of the invention.

In a preferred embodiment, a DISC1 protein of the present invention is a mammalian DISC1 protein. In an even more preferred embodiment, the DISC1 protein is a human protein, such as a DISC1 polypeptide comprising an amino acid sequence from SEQ ID NO: 2 in which amino acid 607 is an phenylalanine residue and/or amino acid 704 is a cysteine.

DISC1 polypeptides are preferably capable of functioning in one of either role of an agonist or antagonist of at least one biological activity of a wild-type (“authentic”) DISC1 protein of the appended sequence listing. The term “evolutionarily related to”, with respect to amino acid sequences of DISC1 proteins, refers to both polypeptides having amino acid sequences which have arisen naturally, and also to mutational variants of human DISC1 polypeptides which are derived, for example, by combinatorial mutagenesis.

Full length proteins or fragments corresponding to one or more particular motifs and/or domains or to arbitrary sizes, for example, at least 5, 10, 25, 50, 75 and 100, amino acids in length are within the scope of the present invention.

Isolated peptidyl portions of DISC1 proteins can be obtained by screening peptides recombinantly produced from the corresponding fragment of the nucleic acid encoding such peptides. In addition, fragments can be chemically synthesized using techniques known in the art such as conventional Merrifield solid phase f-Moc or t-Boc chemistry. For example, a DISC1 polypeptide of the present invention may be arbitrarily divided into fragments of desired length with no overlap of the fragments, or preferably divided into overlapping fragments of a desired length. The fragments can be produced (recombinantly or by chemical synthesis) and tested to identify those peptidyl fragments which can function as either agonists or antagonists of a wild-type (e.g., “authentic”) DISC1 protein.

In general, polypeptides referred to herein as having an activity (e.g., are “bioactive”) of a DISC1 protein are defined as polypeptides which mimic or antagonize all or a portion of the biological/biochemical activities of a DISC1 protein having SEQ ID NO: 2, such as the ability to bind a substrate or ligand. Other biological activities of the subject DISC1 proteins will be reasonably apparent to those skilled in the art. According to the present invention, a polypeptide has biological activity if it is a specific agonist or antagonist of a naturally-occurring form of a DISC1 protein.

Assays for determining whether a DISC1 protein or variant thereof has one or more biological activities are well known in the art.

Other preferred proteins of the invention are those encoded by the nucleic acids set forth in the section pertaining to nucleic acids of the invention. In particular, the invention provides fusion proteins, e.g., DISC1-immunoglobulin fusion proteins. Such fusion proteins can provide, e.g., enhanced stability and solubility of DISC1 proteins and may thus be useful in therapy. Fusion proteins can also be used to produce an immunogenic fragment of a DISC1 protein. For example, the VP6 capsid protein of rotavirus can be used as an immunologic carrier protein for portions of the DISC1 polypeptide, either in the monomeric form or in the form of a viral particle. The nucleic acid sequences corresponding to the portion of a subject DISC1 protein to which antibodies are to be raised can be incorporated into a fusion gene construct which includes coding sequences for a late vaccinia virus structural protein to produce a set of recombinant viruses expressing fusion proteins comprising DISC1 epitopes as part of the virion. It has been demonstrated with the use of immunogenic fusion proteins utilizing the Hepatitis B surface antigen fusion proteins that recombinant Hepatitis B virions can be utilized in this role as well. Similarly, chimeric constructs coding for fusion proteins containing a portion of a DISC1 protein and the poliovirus capsid protein can be created to enhance immunogenicity of the set of polypeptide antigens (see, for example, EP Publication No. 0259149; and Evans et al. (1989) Nature 339:385; Huang et al. (1988) J. Virol. 62:3855; and Schlienger et al (1992) J. Virol. 66:2).

The Multiple antigen peptide system for peptide-based immunization can also be utilized to generate an immunogen, wherein a desired portion of a DISC1 polypeptide is obtained directly from organo-chemical synthesis of the peptide onto an oligomeric branching lysine core (see, for example, Posnett et al. (1988) JBC 263:1719 and Nardelli et al. (1992) J. Immunol 148:914). Antigenic determinants of DISC1 proteins can also be expressed and presented by bacterial cells.

In addition to utilizing fusion proteins to enhance immunogenicity, it is widely appreciated that fusion proteins can also facilitate the expression of proteins, and accordingly, can be used in the expression of the DISC1 polypeptides of the present invention. For example, DISC1 polypeptides can be generated as glutathione-S-transferase (GST-fusion) proteins. Such GST-fusion proteins can enable easy purification of the DISC1 polypeptide, as for example by the use of glutathione-derivatized matrices (see, for example, Current Protocols in Molecular Biology, eds. Ausubel et al. (N.Y.: John Wiley & Sons, 1991)).

The present invention further pertains to methods of producing the subject DISC1 polypeptides. For example, a host cell transfected with a nucleic acid vector directing expression of a nucleotide sequence encoding the subject polypeptides can be cultured under appropriate conditions to allow expression of the peptide to occur. Suitable media for cell culture are well known in the art. The recombinant DISC1 polypeptide can be isolated from cell culture medium, host cells, or both using techniques known in the art for purifying proteins including ion-exchange chromatography, gel filtration chromatography, ultrafiltration, electrophoresis, and immunoaffinity purification with antibodies specific for such peptide. In a preferred embodiment, the recombinant DISC1 polypeptide is a fusion protein containing a domain which facilitates its purification, such as GST fusion protein.

Moreover, it will be generally appreciated that, under certain circumstances, it may be advantageous to provide homologs of one of the subject DISC1 polypeptides which function in a limited capacity as one of either a DISC1 agonist (mimetic) or a DISC1 antagonist, in order to promote or inhibit only a subset of the biological activities of the naturally-occurring form of the protein. Thus, specific biological effects can be elicited by treatment with a homolog of limited function, and with fewer side effects relative to treatment with agonists or antagonists which are directed to all of the biological activities of naturally occurring forms of DISC1 proteins.

Homologs of each of the subject DISC1 proteins can be generated by mutagenesis, such as by discrete point mutation(s), or by truncation. For instance, mutation can give rise to homologs which retain substantially the same, or merely a subset, of the biological activity of the DISC1 polypeptide from which it was derived. Alternatively, antagonistic forms of the protein can be generated which are able to inhibit the function of the naturally occurring form of the protein, such as by competitively binding to a substrate or ligand.

The recombinant DISC1 polypeptides of the present invention also include homologs of DISC1 polypeptides which differ from the DISC1 proteins having SEQ ID NO: 2, such as versions of those protein which are resistant to proteolytic cleavage, as for example, due to mutations which alter ubiquitination or other enzymatic targeting associated with the protein.

DISC1 polypeptides may also be chemically modified to create derivatives by forming covalent or aggregate conjugates with other chemical moieties, such as glycosyl groups, lipids, phosphate, acetyl groups and the like. Covalent derivatives of DISC1 proteins can be prepared by linking the chemical moieties to functional groups on amino acid side-chains of the protein or at the N-terminus or at the C-terminus of the polypeptide.

Modification of the structure of the subject DISC1 polypeptides can be for such purposes as enhancing therapeutic or prophylactic efficacy, stability (e.g., ex vivo shelf life and resistance to proteolytic degradation), or post-translational modifications (e.g., to alter phosphorylation pattern of protein). Such modified peptides, when designed to retain at least one activity of the naturally-occurring form of the protein, or to produce specific antagonists thereof, are considered functional equivalents of the DISC1 polypeptides described in more detail herein. Such modified peptides can be produced, for instance, by amino acid substitution, deletion, or addition. The substitutional variant may be a substituted conserved amino acid or a substituted non-conserved amino acid. For example, it is reasonable to expect that an isolated replacement of a leucine with an isoleucine or valine, an aspartate with a glutamate, a threonine with a serine, or a similar replacement of an amino acid with a structurally related amino acid (i.e. isosteric and/or isoelectric mutations) will not have a major effect on the biological activity of the resulting molecule. Conservative replacements are those that take place within a family of amino acids that are related in their side chains. Genetically encoded amino acids can be divided into four families: (1) acidic=aspartate, glutamate; (2) basic=lysine, arginine, histidine: (3) nonpolar=alanine, valine, leucine, isoleucine, proline, phenylalanine, methionine, tryptophan; and (4) uncharged polar=glycine, asparagine, glutamine, cysteine, serine, threonine, tyrosine. In similar fashion, the amino acid repertoire can be grouped as (1) acidic=aspartate, glutamate; (2) basic=lysine, arginine histidine, (3) aliphatic=glycine, alanine, valine, leucine, isoleucine, serine, threonine, with serine and threonine optionally be grouped separately as aliphatic-hydroxyl; (4) aromatic=phenylalanine, tyrosine, tryptophan; (5) amide=asparagine, glutamine; and (6) sulfur-containing=cysteine and methionine. (see, for example, Biochemistry, 2^{nd} ed., Ed. by L. Stryer, WH Freeman and Co.: 1981). Whether a change in the amino acid sequence of a peptide results in a functional DISC1 homolog (e.g., functional in the sense that the resulting polypeptide mimics or antagonizes the wild-type form) can be readily determined by assessing the ability of the variant peptide to produce a response in cells in a fashion similar to the wild-type protein, or competitively inhibit such a response. Polypeptides in which more than one replacement has taken place can readily be tested in the same manner.

### 5. 4 Expression of DISC1 and DISC2 Gene Products

The nucleotide sequence coding for DISC1 or DISC2, or for an antigenic fragment, derivative or analog thereof, for a functionally active derivative (including a chimeric protein) thereof, or for a mutant or variant DISC1 or DISC2 (such as for one of the variants or mutants described in the Examples, infra) can be inserted into an appropriate expression vector, i.e., a vector which contains the necessary elements for the transcription and translation of the inserted protein-coding sequence. Thus, a nucleic acid encoding DISC1 or DISC2 of the invention can be operationally associated with a promoter in an expression vector of the invention. Both cDNA and genomic sequences can be cloned and expressed under control of such regulatory sequences. Such vectors can be used to express functional or functionally inactivated DISC1 and DISC2 gene products (for example DISC1 and DISC2 RNA or DISC1 polypeptides). These include, for example, a DISC1 or DISC2 nucleic acid having one or more mutations or polymorphisms that are associated wish a neuropsychiatric disorder, such as DISC1 nucleic acids having one or more of the polymorphisms specified in TABLE 5 and in TABLE 6A of the Examples, infra. In addition, nucleic acids that encode a variant DISC1 polypeptide, for example a variant DISC1 polypeptide associated with a neuropsychiatric disorder and/or having one or more of the amino acid substitutions disclosed in TABLE 6B of the Examples, infra, may be cloned and expressed according to the methods described here.

The necessary transcriptional and translational signals can be provided on a recombinant expression vector.

Potential host-vector systems include but are not limited to mammalian cell systems transfected with expression plasmids or infected with virus (e.g., vaccinia virus, adenovirus, adeno-associated virus, herpes virus, etc.); insect cell systems infected with virus (e.g., baculovirus); microorganisms such as yeast containing yeast vectors; or bacteria transformed with bacteriophage, DNA, plasmid DNA, or cosmid DNA. The expression elements of vectors vary in their strengths and specificities. Depending on the host-vector system utilized, any one of a number of suitable transcription and translation elements may be used.

Expression of DISC1 or DISC2 may be controlled by any promoter/enhancer element known in the art, but these regulatory elements must be functional in the host selected for expression. Promoters which may be used to control DISC1 or DISC2 gene expression include, but are not limited to, cytomegalovirus (CMV) promoter (U.S. Pat. Nos. 5,385,839 and No. 5,168,062), the SV40 early promoter region (Benoist and Chambon, 1981, Nature 290:304-310), the promoter contained in the 3′ long terminal repeat of Rous sarcoma virus (Yamamoto, et al., Cell 22:787-797, 1980), the herpes thymidine kinase promoter (Wagner et al., Proc. Natl. Acad. Sci. U.S.A. 78:1441-1445, 1981), the regulatory sequences of the metallothionein gene (Brinster et al., Nature 296:39-42, 1982); prokaryotic expression vectors such as the b-lactamase promoter (Villa-Komaroff, et al., Proc. Natl. Acad. Sci. U.S.A. 75:3727-3731, 1978), or the tac promoter (DeBoer, et al., Proc. Natl. Acad. Sci. U.S.A. 80:21-25, 1983); see also “Useful proteins from recombinant bacteria” in Scientific American, 242:74-94, 1980; promoter elements from yeast or other fungi such as the Gal 4 promoter, the ADC (alcohol dehydrogenase) promoter, PGK (phosphoglycerol kinase) promoter, alkaline phosphatase promoter; and transcriptional control regions that exhibit hematopoietic tissue specificity, in particular: beta-globin gene control region which is active in myeloid cells (Mogram et al., Nature 315:338-340, 1985; Kollias et al., Cell 46:89-94, 1986), hematopoietic stem cell differentiation factor promoters, erythropoietin receptor promoter (Maouche et al., Blood, 15:2557, 1991), etc.

Indeed, any type of plasmid, cosmid, YAC or viral vector may be used to prepare a recombinant nucleic acid construct which can be introduced to a cell, or to tissue, where expression of an a DISC1 or a DISC2 gene product is desired. Alternatively, wherein expression of a recombinant DISC1 or DISC2 gene product in a particular type of cell or tissue is desired, viral vectors that selectively infect the desired cell type or tissue type can be used.

In another embodiment, the invention provides methods for expressing DISC1 polypeptides or DISC2 RNA by using a non-endogenous promoter to control expression of an endogenous DISC1 or DISC2 gene within a cell. An endogenous DISC1 or DISC2 gene within a cell is a gene of the present invention which is ordinarily (i.e., naturally) found in the genome of that cell. A non-endogenous promoter, however, is a promoter or other nucleotide sequence that may be used to control expression of a gene but is not ordinarily or naturally associated with the endogenous DISC1 or DISC2 gene. As an example, methods of homologous recombination may be employed (preferably using non-protein encoding DISC1 or DISC2 nucleic acid sequences of the invention) to insert an amplifiable gene or other regulatory sequence in the proximity of an endogenous DISC1 or DISC2 gene. The inserted sequence may then be used, e.g., to provide for higher levels of DISC1 or DISC2 gene expression than normally occurs in that cell, or to overcome one or more mutations in the endogenous DISC1 or DISC2 regulatory sequences which prevent normal levels of DISC1 or DISC2 gene expression. Such methods of homologous recombination are well known in the art. See, for example, International Patent Publication No. WO 91/06666, published May 16, 1991 by Skoultchi; International Patent Publication No. WO 91/099555, published Jul. 11, 1991 by Chappel; and International Patent Publication No. WO 90/14092, published Nov. 29, 1990 by Kucherlapati and Campbell.

Soluble forms of the protein can be obtained by collecting culture fluid, or solubilizing inclusion bodies, e.g., by treatment with detergent, and if desired sonication or other mechanical processes, as described above. The solubilized or soluble protein can be isolated using various techniques, such as polyacrylamide gel electrophoresis (PAGE), isoelectric focusing, 2-dimensional gel electrophoresis, chromatography (e.g., ion exchange, affinity, immunoaffinity, and sizing column chromatography), centrifugation, differential solubility, immunoprecipitation, or by any other standard technique for the purification of proteins.

A wide variety of host/expression vector combinations may be employed in expressing the DNA sequences of this invention. Useful expression vectors, for example, may consist of segments of chromosomal, non-chromosomal and synthetic DNA sequences. Suitable vectors include derivatives of SV40 and known bacterial plasmids, e.g., E. coli plasmids col E1, pCR1, pBR322, pMal-C2, pET, pGEX (Smith et al., Gene 67:31-40,1988), pMB9 and their derivatives, plasmids such as RP4; phage DNAs, e.g., the numerous derivatives of phage 1, e.g., NM989, and other phage DNA, e.g., M13 and filamentous single stranded phage DNA; yeast plasmids such as the 2 m plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like.

Preferred vectors are viral vectors, such as lentiviruses, retroviruses, herpes viruses, adenoviruses, adeno-associated viruses, vaccinia virus, baculovirus, and other recombinant viruses with desirable cellular tropism. Thus, a gene encoding a functional or mutant DISC1 or DISC2 gene product or a fragment thereof (e.g., a polypeptide domain of a DISC1 or DISC2 gene product) can be introduced in vivo, ex vivo, or in vitro using a viral vector or through direct introduction of DNA. Expression in targeted tissues can be effected by targeting the transgenic vector to specific cells, such as with a viral vector or a receptor ligand, or by using a tissue-specific promoter, or both. Targeted gene delivery is described in International Patent Publication WO 95/28494, published October 1995.

According to the present invention, vectors may be specifically targeted to cells expressing either a DISC1 or a DISC2 gene product (e.g., on their cell surface) using, for example, a DISC1- or DISC2-specific antibody (i.e., an antibody that specifically binds to a DISC1 or a DISC2 gene product), or using a DISC1 or a DISC2 binding partner (for example, a DIS1- or DISC2-specific ligand). Vectors may also be specifically targeted to cells expressing DISC1 or DISC2 using fragments (e.g., peptide or polypeptide fragments) of a DISC1 or DISC2 binding partner, particularly fragments which comprise DISC1 or DISC2 binding sequence. Such methods may be used to target vectors expressing any gene to cells, including but not limited to, vectors that express DISC1 or DISC2 specific antisense nucleic acids, or ribozymes which are specific for DISC1 or DISC2.

Viral vectors commonly used for in vivo or ex vivo targeting and therapy procedures are DNA-based vectors and retroviral vectors. Methods for constructing and using viral vectors are known in the art (see, e.g., Miller and Rosman, BioTechniques, 7:980-990, 1992). Preferably, the viral vectors are replication defective, that is, they are unable to replicate autonomously in the target cell. In general, the genome of the replication defective viral vectors which are used within the scope of the present invention lack at least one region which is necessary for the replication of the virus in the infected cell. These regions can either be eliminated (in whole or in part), be rendered non-functional by any technique known to a person skilled in the art. These techniques include the total removal, substitution (by other sequences, in particular by the inserted nucleic acid), partial deletion or addition of one or more bases to an essential (for replication) region. Such techniques may be performed in vitro (on the isolated DNA) or in site, using the techniques of genetic manipulation or by treatment with mutagenic agents. Preferably, the replication defective virus retains the sequences of its genome which are necessary for encapsidating the viral particles.

DNA viral vectors include an attenuated or defective DNA virus, such as but not limited to herpes simplex virus (HSV), papillomavirus, Epstein Barr virus (EBV). adenovirus, adeno-associated virus (AAV), and the like. Defective viruses, which entirely or almost entirely lack viral genes, are preferred. Defective virus is not infective after introduction into a cell. Use of defective viral sectors allows for administration to cells in a specific, localized area, without concern that the vector can infect other cells. Thus, a specific tissue can be specifically targeted. Examples of particular vectors include, but are not limited to, a defective herpes virus 1 (HSV1) vector (Kaplitt et al., Molec. Cell. Neurosci. 2:320-330, 1991), defective herpes virus vector lacking a glyco-protein L gene (Patent Publication RD 371005 A), or other defective herpes virus vectors (International Patent Publication No. WO 94/21807, published Sep. 29, 1994; International Patent Publication No. WO 92/05263, published Apr. 2, 1994); an attenuated adenovirus vector, such as the vector described by Stratford-Perricaudet et al. (J. Clin. Invest. 90:626-630, 1992; see also La Salle et al., Science 259:988-990, 1993); and a defective adeno-associated virus vector (Samulski et al., J. Virol. 61:3096-3101, 1987; Samulski et al., J. Virol. 63:3822-3828, 1989; Lebkowski et al., Mol. Cell. Biol. 8:3988-3996, 1988).

Various companies produce viral vectors commercially, including but by no means limited to Avigen, Inc. (Alameda, Calif.; AAV vectors), Cell Genesys (Foster City, Calif.; retroviral, adenoviral, AAV vectors, and lentiviral vectors), Clontech (retroviral and baculoviral vectors), Genovo, Inc. (Sharon Hill, Pa.; adenoviral and AAV vectors), Genvec (adenoviral vectors), IntroGene (Leiden, Netherlands; adenoviral vectors), Molecular Medicine (retroviral, adenoviral, AAV, and herpes viral vectors), Norgen (adenoviral vectors), Oxford BioMedica (Oxford, United Kingdom; lentiviral vectors), and Transgene (Strasbourg, France; adenoviral, vaccinia, retroviral, and lentiviral vectors).

In another embodiment, the vector can be introduced in vivo by lipofection, as naked DNA, or with other transfection facilitating agents (peptides, polymers, etc.). Synthetic cationic lipids can be used to prepare liposomes for in vivo transfection of a gene encoding a marker (Felgner, et. al., Proc. Natl. Acad. Sci. U.S.A. 84:7413-7417, 1987; Felgner and Ringold, Science 337:387-388, 1989; see Mackey, et al., Proc. Natl. Acad. Sci. U.S.A. 85:8027-8031, 1988; Ulmer et al., Science 259:1745-1748, 1993). Useful lipid compounds and compositions for transfer of nucleic acids are described in International Patent Publications WO95/18863 and WO96/17823, and in U.S. Pat. No. 5,459,127. Lipids may be chemically coupled to other molecules for the purpose of targeting (see Mackey, et. al., supra). Targeted peptides, e.g., hormones or neurotransmitters, and proteins such as antibodies, or non-peptide molecules could be coupled to liposomes chemically. Other molecules are also useful for facilitating transfection of a nucleic acid in vivo, such as a cationic oligopeptide (e.g., International Patent Publication WO95/21931), peptides derived from DNA binding proteins (e.g., International Patent Publication WO96/25508), or a cationic polymer (e.g., International Patent Publication WO95/21931).

It is also possible to introduce the vector in vivo as a naked DNA plasmid. Naked DNA vectors for gene therapy can be introduced into the desired host cells by methods known in the art, e.g., electroporation, microinjection, cell fusion, DEAE dextran, calcium phosphate precipitation, use of a gene gun, or use of a DNA vector transporter (see, e.g., Wu et al., J. Biol. Chem. 267:963-967, 1992; Wu and Wu, J. Biol. Chem. 263:14621-14624, 1988; Hartmut et al., Canadian Patent Application No. 2,012,311, filed Mar. 15, 1990; Williams et al., Proc. Natl. Acad. Sci. USA 88:2726-2730, 1991). Receptor-mediated DNA delivery approaches can also be used (Curiel et al., Hum. Gene Ther. 3:147-154, 1992; Wu and Wu, J. Biol. Chem. 262:4429-4432, 1987). U.S. Pat. Nos. 5,580,859 and 5,589,466 disclose delivery of exogenous DNA sequences, free of transfection facilitating agents, in a mammal. Recently, a relatively low voltage, high efficiency in vivo DNA transfer technique, termed electrotransfer, has been described (Mir et al., C. P. Acad. Sci., 321:893, 1998; WO 99/01157; WO 99/01158; WO 99/01175).

Preferably, for in vivo administration, an appropriate immunosuppressive treatment is employed in conjunction with the viral vector, e.g., adenovirus vector, to avoid immuno-deactivation of the viral vector and transfected cells. For example, immunosuppressive cytokines, such as interleukin-12 (IL-12), interferon-g (IFN-γ), or anti-CD4 antibody, can be administered to block humoral or cellular immune responses to the viral vectors (see, e.g., Wilson, Nature Medicine, 1995). In that regard, it is advantageous to employ a viral vector that is engineered to express a minimal number of antigens.

### 5.5 Antibodies to DISC1 and DISC2

Antibodies to DISC1 and DISC2 are useful, inter alia, for diagnostics and intracellular regulation of DISC1 and DISC2 activity, as set forth below. For example, such antibodies may be used in the methods and compositions of this invention to diagnose and/or treat disorders, including neuropsychiatric disorders (for example, schizophrenia, schizoaffective disorder, bipolar affective disorder, unipolar affective disorder and adolescent conduct disorder) associated with either DISC1, DISC2 or both DISC1 and DISC2.

According to the invention, DISC1 and DISC2 polypeptides and/or nucleic acids that are produced recombinantly or by chemical synthesis, as well as fragments or other derivatives or analogs thereof (including fusion proteins), may be used as an immunogen to generate antibodies that recognize DISC1 or DISC2. Such antibodies include but are not limited to polyclonal, monoclonal, chimeric, single chain, Fab fragments, and an Fab expression library. Such an antibody is specific for human DISC1 or DISC2; it may recognize a mutant form of DISC1 or DISC2, a wild-type DISC1 or DISC2, or both.

For example, in preferred embodiments an antibody may recognize and specifically bind to a mutant or variant DISC1 or DISC2. Such an antibody may recognize and specifically bind to a DISC1 or DISC2 gene product (e.g., a polypeptide or nucleic acid) encoded by a DISC1 or DISC2 nucleic acid that has at least one polymorphism (e.g., at least one of the specific DISC1 and DISC2 polymorphisms described in the Examples, infra).

Preferably, such an antibody recognizes and specifically binds to a gene product encoded by a variant DISC1 or DISC2 nucleic acid but does not bind to a gene product encoded by a wild-type DISC1 or DISC2 nucleic acid (e.g., a gene product encoded by any of the DISC1 and DISC2 nucleic acids set forth in FIGS. 1A-C and in FIGS. 3A-G, respectively, and in SEQ ID NOS:1 and 3).

Various procedures known in the art may be used for the production of polyclonal antibodies to OSCAR polypeptide or derivative or analog thereof. For the production of antibody, various host animals can be immunized by injection with the OSCAR polypeptide, or a derivative (e.g., fragment or fusion protein) thereof, including but not limited to rabbits, mice, rats, sheep, goats, etc. In one embodiment, a DISC1 or DISC2 polypeptide or fragment thereof can be conjugated to an immunogenic carrier, e.g., bovine serum albumin (BSA) or keyhole limpet hemocyanin (KLH). Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum.

For preparation of monoclonal antibodies directed toward the DISC1 or DISC2 gene product, or a fragment, analog, or derivative thereof, any technique that provides for the production of antibody molecules by continuous cell lines in culture may be used. These include but are not limited to the hybridoma technique originally developed by Kohler and Milstein (Nature 1975, 256:495-497), as well as the trioma technique, the human B-cell hybridoma technique (Kozbor et al., Immunology Today 1983, 4:72; Cote et al., Proc. Natl. Acad. Sci. U.S.A. 1983, 80:2026-2030), and the EBV-hybridoma technique to produce human monoclonal antibodies (Cole et al., in Monoclonal Antibodies and Cancer Therapy, Alan R. Liss, Inc., 1985, pp. 77-96). In an additional embodiment of the invention, monoclonal antibodies can be produced in germ-free animals (International Patent Publication No. WO 89/12690). In fact, according to the invention, techniques developed for the production of “chimeric antibodies” (Morrison et al., J. Bacteriol. 1984, 159:870; Neuberger et al., Nature 1984, 312:604-608; Takeda et al., Nature 1985, 314:452-454) by splicing the genes from a mouse antibody molecule specific for an OSCAR polypeptide together with genes from a human antibody molecule of appropriate biological activity can be used; such antibodies are within the scope of this invention. Such human or humanized chimeric antibodies are preferred for use in therapy of human diseases or disorders (described infra), since the human or humanized antibodies are much less likely than xenogenic antibodies to induce an immune response, in particular an allergic response, themselves.

Antibody fragments which contain the idiotype of the antibody molecule can be generated by known techniques. For example, such fragments include but are not limited to: the F(ab′)₂ fragment which can be produced by pepsin digestion of the antibody molecule; the Fab′ fragments which can be generated by reducing the disulfide bridges of the F(ab′)₂ fragment, and the Fab fragments which can be generated by treating the antibody molecule with papain and a reducing agent.

According to the invention, techniques described for the production of single chain antibodies (U.S. Pat. Nos. 5,476,786, 5,132,405, and 4,946,778) can be adapted to produce DISC1- or DISC2-specific single chain antibodies. An additional embodiment of the invention utilizes the techniques described for the construction of Fab expression libraries (Huse et al., Science 1989, 246:1275-1281) to allow rapid and easy identification of monoclonal Fab fragments with the desired specificity for DISC1 or DISC2, or for its derivatives or analogs.

In the production and use of antibodies, screening for or testing with the desired antibody can be accomplished by techniques known in the art, e.g., radioimmunoassay, ELISA (enzyme-linked immunosorbant assay), “sandwich” immunoassays, immunoradiometric assays, gel diffusion precipitin reactions, immunodiffusion assays, in situ immunoassays (using colloidal gold, enzyme or radioisotope labels, for example), western blots, precipitation reactions, agglutination assays (e.g., gel agglutination assays, hemagglutination assays), complement fixation assays, immunofluorescence assays, protein A assays, and immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention. For example, to select antibodies which recognize a specific epitope of a DISC1 or DISC2 gene product, one may assay generated hybridomas for a product which binds to a DISC1 or DISC2 fragment containing such epitope. For selection of an antibody specific to either a DISC1 or a DISC2 gene product from a particular species of animal, one can select on the basis of positive binding with a DISC1 or a DISC2 gene product expressed by or isolated from cells of that species of animal.

The foregoing antibodies can be used in methods known in the art relating to the localization and activity of a DISC1 or a DISC2 gene product, such as a polypeptide. For example, the antibodies may be used for Western blotting, imaging DISC1 or DISC2 in situ, measuring levels thereof in appropriate physiological samples, etc. using any of the detection techniques mentioned above or known in the art. Such antibodies can also be used in assays for ligand binding, e.g., as described in U.S. Pat. No. 5,679,582. Antibody binding generally occurs most readily under physiological conditions, e.g., pH of between about 7 and 8, and physiological ionic strength. The presence of a carrier protein in the buffer solutions stabilizes the assays. While there is some tolerance of perturbation of optimal conditions, e.g., increasing or decreasing ionic strength, temperature, or pH, or adding detergents or chaotropic salts, such perturbations will decrease binding stability.

In still other embodiments, anti-DISC1 and/or anti-DISC2 antibodies may also be used to isolate cells which express either DISC1 or DISC2, by panning or related immunoadsorption techniques.

In a specific embodiment, antibodies that agonize or antagonize the activity of DISC1 or DISC2 can be generated. In particular, intracellular single chain Fv antibodies can be used to regulate (inhibit) DISC1 or DISC2 activity (Marasco et al., Proc. Natl. Acad. Sci. U.S.A. 1993, 90:7884-7893; Chen., Mol. Med. Today 1997, 3:160-167; Spitz et al., Anticancer Res. 1996, 16:3415-22; Indolfi et al., Nat. Med. 1996, 2:634-635; Kijma et al., Pharmacol. Ther. 1995, 68:247-267). Such antibodies can be tested using the assays described infra for identifying ligands.

### 5.6 In Vivo Testing Using Transgenic Animals

Transgenic animals, including transgenic mammals, may be prepared for evaluating the molecular mechanism(s) of DISC1 or I)ISC2 and, particularly, for evaluating the molecular mechanism(s) of disease and disorders, for example neuropsychiatric disorders (e.g., schizophrenia, schizoaffective disorder, bipolar affective disorder, unipolar affective disorder and adolescent conduct disorder), that are associated with DISC1 or DISC2. Such animals provide excellent models for screening and/or testing drug candidates for such disorders. Thus, human DISC1 and/or DISC2 “knock-in” animals, including human DISC1 and/or DISC2 “knock-in” mammals, can be prepared for evaluating the molecular biology to this system in greater detail than is possible with human subjects. It is also possible to evaluate compounds or diseases in “knockout” animals, e.g., to identify a compound that can compensate for a defect in DISC1 or DISC2 activity. Both technologies permit manipulation of single units of genetic information in their natural position in a cell genome and to examine the results of that manipulation in the background of a terminally differentiated organism. Trangenic mammals can be prepared by any method, including but not limited to modification of embryonic stem (ES) cells and heteronuclear injecion into blast cells.

A “knock-in” animal is an animal (e.g., a mammal such as a mouse) in which an endogenous gene is substituted with a heterologous gene (Roamer et al., New Biol. 1991, 3:331). Preferably, the heterologous gene is “knocked-in” to a locus of interest, either the subject of evaluation (in which case the gene may be a reporter gene; see Elegant et al., Proc. Natl. Acad. Sci. USA 1998, 95:11897) of expression or function of a homologous gene, thereby linking the heterologous gene expression to transcription from the appropriate promoter. This can be achieved by homologous recombination, transposon (Westphal and Leder, Curr Biol 1997, 7:530), using mutant recombination sites (Araki et al., Nucleic Acids Res 1997, 25:868) or PCR (Zhang and Henderson, Biotechniques 1998, 25:784).

A “knockout animal” is an animal (e.g., a mammal such as a mouse) that contains within its genome a specific gene that has been inactivated by the method of gene targeting (see, e.g., U.S. Pat. Nos. 5,777,195 and 5,616,491). A knockout animal includes both a heterozygote knockout (i.e., one defective allele and one wild-type allele) and a homozygous mutant. Preparation of a knockout animal requires first introducing a nucleic acid construct that will be used to suppress expression of a particular gene into an undifferentiated cell type termed an embryonic stem cell. This cell is then injected into a mammalian embryo. In preferred embodiments for which the knockout animal is a mammal, a mammalian embryo with an integrated cell is then implanted into a foster mother for the duration of gestation. Zhou, et al. (Genes and Development, 1995, 9:2623-34) describes PPCA knock-out mice.

The term “knockout” refers to partial or complete suppression of the expression of at least a portion of a protein encoded by an endogenous DNA sequence in a cell. The term “knockout construct” refers to a nucleic acid sequence that is designed to decrease or suppress expression of a protein encoded by endogenous DNA sequences in a cell. The nucleic acid sequence used as the knockout construct is typically comprised of: (1) DNA from some portion of the gene (exon sequence, intron sequence, and/or promoter sequence) to be suppressed; and (2) a marker sequence used to detect the presence of the knockout construct in the cell. The knockout construct is inserted into a cell, and integrates with the genomic DNA of the cell in such a position so as to prevent or interrupt transcription of the native DNA sequence. Such insertion usually occurs by homologous recombination (i.e., regions of the knockout construct that are homologous to endogenous DNA sequences hybridize to each other when the knockout construct is inserted into the cell and recombine so that the knockout construct is incorporated into the corresponding position of the endogenous DNA). The knockout construct nucleic acid sequence may comprise: (1) a full or partial sequence of one or more exons and/or introns of the gene to be suppressed; (2) a full or partial promoter sequence of the gene to be suppressed; or (3) combinations thereof. Typically, the knockout construct is inserted into an embryonic stem cell (ES cell) and is integrated into the ES cell genomic DNA, usually by the process of homologous recombination. This ES cell is then injected into, and integrates with, the developing embryo.

The phrases “disruption of the gene” and “gene disruption” refer to insertion of a nucleic acid sequence into one region of the native DNA sequence (usually one or more exons) and/or the promoter region of a gene so as to decrease or prevent expression of that gene in the cell as compared to the wild-type or naturally occurring sequence of the gene. By way of example, a nucleic acid construct can be prepared containing a DNA sequence encoding an antibiotic resistance gene which is inserted into the DNA sequence that is complementary to the DNA sequence (promoter and/or coding region) to be disrupted. When this nucleic acid construct is then transfected into a cell, the construct will integrate into the genomic DNA Thus, many progeny of the cell will no longer express the gene at least in some cells, or will express it at a decreased level, as the DNA is now disrupted by the antibiotic resistance gene.

Generally, for homologous recombination, the DNA will be at least about 1 kilobase (kb) in length and preferably 3-4 kb in length, thereby providing sufficient complementary sequence for recombination when the knockout construct is introduced into the genomic DNA of the ES cell (discussed below).

Included within the scope of this invention is an animal, preferably a mammal (e.g., a mouse) in which two or more genes have been knocked out or knocked in, or both. Such animals can be generated by repeating the procedures set forth herein for generating each knockout construct, or by breeding two animals, each with a single gene knocked out, to each other, and screening for those with the double knockout genotype.

Regulated knockout animals can be prepared using various systems, such as the tet-repressor system (see U.S. Pat. No. 5,654,168) or the Cre-Lox system (see U.S. Pat. Nos. 4,959,317 and No. 5,801,030).

In another series of embodiments, transgenic animals are created in which: (i) a human DISC1 and/or DISC2 gene(s) is(are) stably inserted into the genome of the transgenic animal; and/or (ii) the endogenous DISC1 and/or DISC2 genes are inactivated and replaced with their human counterparts (see, e.g., Coffman, Semin. Nephrol. 1997, 17:404; Esther et al., Lab. Invest. 1996, 74:953; Murakami et al., Blood Press. Suppl. 1996, 2:36). In one aspect of these embodiments, the human DISC1 and/or DISC2 genes inserted into and/or expressed by the transgenic animal comprise a wild-type DISC1 or DISC2 gene. For example, the wild-type human DISC1 gene may be a gene that encodes a polypeptide having the amino acid sequence set forth in FIG. 2 (SEQ ID NO:2). The wild-type human DISC2 gene may be a gene that encodes a nucleic acid gene product having the sequence shown in FIGS. 3A-G (SEQ ID NO:3). In another aspect of these embodiments, the human DISC1 and/or DISC2 genes inserted into and/or expressed by the transgenic animal comprise a mutant or variant DISC1 or DISC2 gene. For example, a DISC1 or DISC2 gene having one or more of the polymorphisms described in the Examples infra may be inserted into and/or expressed by a transgenic animal of the invention. In a particularly preferred aspect of these embodiments, the polymorphism or mutation is one that is associated with a neuropsychiatric disorder such as schizophrenia, schizoaffective disorder, bipolar affective disorder, unipolar affective disorder and adolescent conduct disorder.

Such transgenic animals can be treated with candidate compounds and monitored for neuronal development, neurodegeneration, or efficacy of a candidate therapeutic compound.

### 5.7 Applications and Uses

Described herein are various applications and uses for DISC1 and DISC2 gene sequences (including fragments of full length DISC1 and DISC2 gene sequences), DISC1 and DISC2 polypeptides (including fragments of full length DISC1 and DISC2 proteins, as well as DISC1 and DISC2 fusion polypeptides) and of antibodies directed against DISC1 or DISC2 nucleic acids, and antibodies directed against DISC1 or DISC2 polypeptides (including fragments of full length DISC1 and DISC2 genes and proteins). Such applications may include, for example, both prognostic and diagnostic applications for evaluating neuropsychiatric disorders (for example, schizophrenia, schizoaffective disorder, bipolar affective disorder, unipolar affective disorder and adolescent conduct disorder) associated with a DISC1 or DISC2 gene and/or with a DISC1 or DISC2 gene product or polypeptide, including the identification of subjects having such a disorder or having a predisposition to such a disorder. Additionally, such applications may include methods for treating disorders associated with a DISC1 or DISC2 gene, with a DISC1 or DISC2 gene product, or with DISC1 or DISC2 polypeptide, as well as screening methods to identify compounds (including natural ligands and other cellular compounds) that modulate the synthesis, expression or activity of either a DISC1 or DISC2 gene, a DISC1 or DISC2 gene product, a DISC1 or DISC2 polypeptide, or a combination thereof.

As explained supra, the DISC1 and DISC2 genes and their gene products are associated with neuropsychiatric disorders such as schizophrenia, schizoaffective disorder, bipolar disorder, unipolar affective disorder and adolescent conduct disorder. In addition, the Examples infra provide novel mutations and polymorphisms in the DISC1 and DISC2 genes which are associated with such neuropsychiatric disorders. The Examples also describe variant DISC1 and DISC2 gene products that are encoded by these altered DISC1 and DISC2 genes and therefore are also associated with such neuropsychiatric disorders.

Accordingly, in preferred embodiments the DISC1 and DISC2 nucleic acids and polypeptides of the invention, as well as antibodies directed against such DISC1 and DISC2 nucleic acids and polypeptides, may be used: (1) in prognostic and diagnostic applications to identify individuals having a neuropsychiatric disorder (for example, schizophrenia, schizoaffective disorder, bipolar affective disorder, unipolar affective disorder and adolescent conduct disorder) or having a predisposition to such a neuropsychiatric disorder; and (2) in screening methods for identifying compounds (including natural ligands and other cellular compounds, as well as synthetic chemical compounds) that modulate activity of DISC1 and/or DISC2 genes or gene products and may be used, therefore, to treat a neuropsychiatric disorder such as schizophrenia, schizoaffective disorder, bipolar affective disorder, unipolar affective disorder and adolescent conduct disorder.

### Prognostic and Diagnostic Assays

The present methods provide means for determining if a subject has (diagnostic) or is at risk of developing (prognostic) a disease, condition or disorder that is associated with a DISC1 or DISC2 allele, e.g., neuropsychiatric disorders such as schizophrenia, ADD, schizoaffectiove disorder, BAD, unipolar affective disorder, and adolescent conduct disorder, or a neuropsychiatric disease or disorder/disorders resulting therefrom.

The present invention provides methods for determining the molecular structure of a DISC1 or DISC2 gene, such as a human DISC1 or DISC2 gene or a portion thereof. In one embodiment, determining the molecular structure of at least a portion of a DISC1 or DISC2 gene comprises determining the identity of the allelic variant of at least one polymorphic region of the gene (determining the presence or absence of one or more of the allelic variants, or their complements, of SEQ ID NOs.:1-4, and/or the sequences set forth in TABLE 3). A polymorphic region of the DISC1 or DISC2 gene can be located in an exon, an intron, at an intron/exon border, or in the promoter of the gene.

The invention provides methods for determining whether a subject has, or is at risk of developing, a disease or condition associated with a specific allelic variant of a polymorphic region of a DISC1 or DISC2 gene. Such diseases can be associated with an abnormal neurological activity, such as, e.g., those associated with the onset of a neuropsychiatric disorder such as schizophrenia, schizoaffective disorder, bipolar disorder, unipolar affective disorder and adolescent conduct disorder. An aberrant DISC1 protein level can result from an aberrant transcription or post-transcriptional regulation. Thus, allelic differences in specific regions of a DISC1 gene can result in differences in the encoded protein due to differences in regulation of expression. In particular, some of the identified polymorphisms in the human DISC1 or DISC2 gene may be associated with differences in the level of transcription, RNA maturation, splicing, or translation of the gene or transcription product.

Analysis of one or more DISC1 or DISC2 polymorphic region in a subject can be useful for predicting whether a subject has or is likely to develop aberrant neurological activities or disorders resulting therefrom, such as neuropsychatric disorders or diseases, e.g., schizophrenia, ADD, schizoaffectiove disorder, BAD, unipolar affective disorder, and adolescent conduct disorder.

In preferred embodiments, the methods of the invention can be characterized as comprising detecting, in a sample of cells from the subject, the presence or absence of a specific allelic variant of one or more polymorphic regions of a DISC1 or DISC2 gene. The allelic differences can be: (i) a difference in the identity of at least one nucleotide or (ii) a difference in the number of nucleotides, which difference can be a single nucleotide or several nucleotides. The invention also provides methods for detecting differences in DISC1 or DISC2 genes such as chromosomal rearrangements, e.g., chromosomal dislocation. The invention can also be used in prenatal diagnostics.

A preferred detection method is allele specific hybridization using probes overlapping the polymorphic site and having about 5, 10, 20, 25, or 30 nucleotides around the polymorphic region. Examples of probes for detecting specific allelic variants of a polymorphic region located in the DISC1 gene are nucleic acid sequences comprising a nucleotide sequence from any of SEQ ID NOS: 33-43, as set forth in TABLE 5A and 5B, infra. For instance, a probe for detecting a specific allelic variant in exon 9 is set forth in SEQ ID NO:33; a probe for detecting specific allelic variants of the polymorphic region located in intron 10 is set forth in SEQ ID NO:34; a probe for detecting specific allelic variants of the polymorphic region located in exon 11 is set forth in SEQ ID NO:35; probes for detecting specific allelic variants of the polymorphic region located in exon 13 is set forth in any of SEQ ID NOS:36-41; a probe for detecting a specific allelic variant of the 5′-promoter is provided in SEQ ID NO:42; and a probe for detecting a specific allelic variant of a polymorphic region located in exon 6 is set forth in SEQ ID NO:43. In a preferred embodiment of the invention, several probes capable of hybridizing specifically to allelic variants are attached to a solid phase support, e.g., a “chip”. Oligonucleotides can be bound to a solid support by a variety of processes, including lithography. For example a chip can hold up to 250,000 oligonucleotides (GeneChip, Affymetrix). Mutation detection analysis using these chips comprising oligonucleotides, also termed “DNA probe arrays” is described e.g., in Cronin et al. (1996) Human Mutation 7:244. In one embodiment, a chip comprises all the allelic variants of at least one polymorphic region of a gene. The solid phase support is then contacted with a test nucleic acid and hybridization to the specific probes is detected. Accordingly, the identity of numerous allelic variants of one or more genes can be identified in a simple hybridization experiment. For example, the identity of the allelic variant of the nucleotide polymorphism in the 5′ promoter region can be determined in a single hybridization experiment.

In other detection methods, it is necessary to first amplify at least a portion of the DISC1 or DISC2 gene prior to identifying the allelic variant. Amplification can be performed, e.g., by PCR and/or LCR (see Wu and Wallace, (1989) Genomics 4:560). according to methods known in the art. In one embodiment, genomic DNA of a cell is exposed to two PCR primers and amplification for a number of cycles sufficient to produce the required amount of amplified DNA. In preferred embodiments, the primers are located between 150 and 350 base pairs apart. Preferred primers, such as primers for amplifying each of the exons of the human DISC1 or DISC2 gene, are listed in FIG. 4.

Alternative amplification methods include: self sustained sequence replication (Guatelli, J. C. et al., 1990, Proc. Natl. Acad. Sci. USA 87:1874-1878), transcriptional amplification system (Kwoh, D. Y. et al., 1989, Proc. Natl. Acad. Sci. USA 86:1173-1177), Q-Beta Replicase (Lizardi, P. M. et al., 1988, Bio/Technology 6:1197), and self-sustained sequence replication (Guatelli et al., (1989) Proc. Nat. Acad. Sci. 87:1874), and nucleic acid based sequence amplification (NABSA), or any other nucleic acid amplification method, followed by the detection of the amplified molecules using techniques well known to those of skill in the art. These detection schemes are especially useful for the detection of nucleic acid molecules if such molecules are present in very low numbers.

In one embodiment, any of a variety of sequencing reactions known in the art can be used to directly sequence at least a portion of a DISC1 or DISC2 gene and detect allelic variants, e.g., mutations, by comparing the sequence of the sample sequence with the corresponding wild-type (control) sequence. Exemplary sequencing reactions include those based on techniques developed by Maxam and Gilbert (Proc. Natl Acad Sci USA (1977) 74:560) or Sanger (Sanger et al (1977) Proc. Nat. Acad. Sci 74:5463). It is also contemplated that any of a variety of automated sequencing procedures may be utilized when performing the subject assays (Biotechniques (1995) 19:448), including sequencing by mass spectrometry (see, for example, U.S. Pat. No. 5,547,835 and international patent application Publication Number WO 94/16101, entitled DNA Sequencing by Mass Spectrometry by H. Köster; U.S. Pat. No. 5,547,835 and international patent application Publication Number WO 94/21822 entitled “DNA Sequencing by Mass Spectrometry Via Exonuclease Degradation” by H. Köster), and U.S. Pat. No. 5,605,798 and International Patent Application No. PCT/US96/03651 entitled DNA Diagnostics Based on Mass Spectrometry by H. Köster;. Cohen et al. (1996) Adv Chromatogr 36:127-162; and Griffin et al. (1993) Appl Biochem Biotechnol 38:147-159). It will be evident to one skilled in the art that, for certain embodiments, the occurrence of only one, two or three of the nucleic acid bases need be determined in the sequencing reaction. For instance, A-track or the like, e.g., where only one nucleotide is detected, can be carried out.

Yet other sequencing methods are disclosed, e.g., in U.S. Pat. No. 5,580,732 entitled “Method of DNA sequencing employing a mixed DNA-polymer chain probe” and U.S. Pat. No. 5,571,676 entitled “Method for mismatch-directed in vitro DNA sequencing”.

In some cases, the presence of a specific allele of a DISC1 or DISC2 gene in DNA from a subject can be shown by restriction enzyme analysis. For example, a specific nucleotide polymorphism can result in a nucleotide sequence comprising a restriction site which is absent from the nucleotide sequence of another allelic variant. Thus, polymorphisms can be determined by analyzing the products or restriction digests.

In a further embodiment, protection from cleavage agents (such as a nuclease, hydroxylamine or osmium tetroxide and with piperidine) can be used to detect mismatched bases in RNA/RNA DNA/DNA, or RNA/DNA heteroduplexes (Myers, et al. (1985) Science 230:1242). In general, the technique of “mismatch cleavage” starts by providing heteroduplexes formed by hybridizing a control nucleic acid, which is optionally labeled, e.g., RNA or DNA, comprising a nucleotide sequence of a 5-LO allelic variant with a sample nucleic acid, e.g, RNA or DNA, obtained from a tissue sample. The double-stranded duplexes are treated with an agent which cleaves single-stranded regions of the duplex such as duplexes formed based on base-pair mismatches between the control and sample strands. For instance, RNA/DNA duplexes can be treated with RNase and DNA/DNA hybrids treated with S1 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine whether the control and sample nucleic acids have an identical nucleotide sequence or in which nucleotides they are different. See, for example, Cotton et al (1988) Proc. Natl Acad Sci USA 85:4397; Saleeba et al (1992) Methods Enzymod. 217:286-295. In a preferred embodiment, the control or sample nucleic acid is labeled for detection.

In another embodiment, an allelic variant can be identified by denaturing high-performance liquid chromatography (DHPLC; see Examples, infra) (Oefner and Underhill, (1995) Am. J. Human Gen. 57:Suppl. A266). In general, PCR products are produced using PCR primers flanking the DNA of interest. DHPLC analysis is carried out and the resulting chromatograms are analyzed to identify base pair alterations or deletions based on specific chromatographic profiles (see O'Donovan et al. (1998) Genomics 52:44-49).

In other embodiments, alterations in electrophoretic mobility is used to identify the type of DISC1 or DISC2 allelic variant. For example, single strand conformation polymorphism (SSCP; see Examples, infra) may be used to detect differences in electrophoretic mobility between mutant and wild type nucleic acids (Orita et al. (1989) Proc Natl. Acad. Sci USA 86:2766, see also Cotton (1993) Mutat Res 285:125-144; and Hayashi (1992) Genet Anal Tech Appl 9:73-79). Single-stranded DNA fragments of sample and control nucleic acids are denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In another preferred embodiment, the subject method utilizes heteroduplex analysis to separate double stranded heteroduplex molecules on the basis of changes in electrophoretic mobility (Keen et al. (1991) Trends Genet 7:5).

In yet another embodiment, the identity of an allelic variant of a polymorphic region is obtained by analyzing the movement of a nucleic acid comprising the polymorphic region in polyacrylamide gels containing a gradient of denaturant is assayed using denaturing gradient gel electrophoresis (DGGE) (Myers et al (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a Temperature gradient is used in place of a denaturing agent gradient to identify differences in the mobility of control and sample DNA (Rosenbaum and Reissner (1987) Biophys Chem 265.1275).

Examples of techniques for detecting differences of at least one nucleotide between 2 nucleic acids include, but are not limited to, selective oligonucleotide hybridization, selective amplification, or selective primer extension. For example, oligonucleotide probes may be prepared in which the known polymorphic nucleotide is placed centrally (allele-specific probes) and then hybridized to target DNA under conditions which permit hybridization only if a perfect match is found (Saiki et al. (1986) Nature 324:163); Saiki et al (1989) Proc. Natl Acad. Sci USA 86:6230; and Wallace et al. (1979) Nucl. Acids Res. 6:3543). Such allele specific oligonucleotide hybridization techniques may be used for the simultaneous detection of several nucleotide changes in different polymorphic regions of the DISC1 or DISC2 gene. For example, oligonucleotides having nucleotide sequences of specific allelic variants are attached to a hybridizing membrane and this membrane is then hybridized with labeled sample nucleic acid. Analysis of the hybridization signal will then reveal the identity of the nucleotides of the sample nucleic acid.

Alternatively, allele specific amplification technology which depends on selective PCR amplification may be used in conjunction with the instant invention. Oligonucleotides used as primers for specific amplification may carry the allelic variant of interest in the center of the molecule (so that amplification depends on differential hybridization) (Gibbs et al (1989) Nucleic Acids Res. 17:2437-2448) or at the extreme 3′ end of one primer where, under appropriate conditions, mismatch can prevent, or reduce polymerase extension (Prossner (1993) Tibtech 11:238; Newton et al. (1989) Nucl. Acids Res. 17:2503). This technique is also termed “PROBE” for Probe Oligo Base Extension. In addition it may be desirable to introduce a novel restriction site in the region of the mutation to create cleavage-based detection (Gasparini et al (1992) Mol Cell Probes 6:1).

In another embodiment, identification of the allelic variant is carried out using an oligonucleotide ligation assay (OLA), as described, e.g., in U.S. Pat. No. 4,998,617 and in Landegren, U. et al., (1988) Science 241:1077-1080 The OLA protocol uses two oligonucleotides which are designed to be capable of hybridizing to abutting sequences of a single strand of a target. One of the oligonucleotides is linked to a separation marker, e.g,. biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand. Nickerson, D. A. et al. have described a nucleic acid detection assay that combines attributes of PCR and OLA (Nickerson, D. A. et al., (1990) Proc. Natl. Acad. Sci. (U.S.A.) 87:8923-8927. In this method, PCR is used to achieve the exponential amplification of target DNA, which is then detected using OLA.

Several techniques based on this OLA method have been developed and can be used to detect specific allelic variants of a polymorphic region of a DISC1 or DISC2 gene. For example, U.S. Pat. No. 5,593,826 discloses an OLA using an oligonucleotide having 3′-amino group and a 5′-phosphorylated oligonucleotide to form a conjugate having a phosphoramidate linkage. In another variation of OLA described in Tobe et al. ((1996) Nucleic Acids Res 24: 3728), OLA combined with PCR permits typing of two alleles in a single microtiter well. By marking each of the allele-specific primers with a unique hapten, i.e. digoxigenin and fluorescein, each OLA reaction can be detected by using hapten specific antibodies that are labeled with different enzyme reporters, alkaline phosphatase or horseradish peroxidase. This system permits the detection of the two alleles using a high throughput format that leads to the production of two different colors.

The invention further provides methods for detecting single nucleotide polymorphisms (SNPs) in a DISC1 or DISC2 gene. Because single nucleotide polymorphisms constitute sites of variation flanked by regions of invariant sequence, their analysis requires no more than the determination of the identity of the single nucleotide present at the site of variation and it is unnecessary to determine a complete gene sequence for each patient. Several methods have been developed to facilitate the analysis of such single nucleotide polymorphisms.

In one embodiment, the single base polymorphism can be detected by using a specialized exonuclease-resistant nucleotide, as disclosed, e.g., in Mundy, C. R. (U.S. Pat. No. 4,656,127). According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amounts of extraneous sequence data.

In another embodiment of the invention, a solution-based method is used for determining the identity of the nucleotide of a polymorphic site. Cohen, D. et al. (French Patent 2,650,840; PCT Appln. No. WO91/02087). As in the Mundy method of U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

An alternative method, known as Genetic Bit Analysis (“GBA”) is described by Goelet, P. et al. (PCT Appln. No. 92/15712). The method of Goelet, P. et al. uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Appln. No. WO91/02087) the method of Goelet, P. et al. is preferably a heterogeneous phase assay, in which the primer or the target molecule is immobilized to a solid phase.

Recently, several primer-guided nucleotide incorporation procedures for assaying polymorphic sites in DNA have been described (Komher, J. S. et al., Nucl. Acids. Res. 17:7779-7784 (1989); Sokolov, B. P., Nucl. Acids Res. 18:3671 (1990); Syvanen, A. -C., et al., Genomics 8:684-692 (1990); Kuppuswamy, M. N. et al., Proc. Natl. Acad. Sci. (U.S.A.) 88:1143-1147 (1991); Prezant, T. R. et al., Hum. Mutat. 1:159-164 (1992); Ugozzoli, L. et al., GATA 9:107-112 (1992); Nyren, P. et al., Anal. Biochem. 208:171-175 (1993)). These methods differ from GBAÔ in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A. -C., et al., Amer. J. Hum. Genet. 52:46-59 (1993)).

For determining the identity of the allelic variant of a polymorphic region located in the coding region of a DISC1 or DISC2 gene, yet other methods than those described above can be used. For example, identification of an allelic variant which encodes a mutated DISC1 protein can be performed by using an antibody specifically recognizing the mutant protein in, e.g., immunohistochemistry or immunoprecipitation. Antibodies to wild-type DISC1 protein or mutated forms of DISC1 proteins can be prepared according to methods known in the art and are also described herein. Preferred antibodies specifically bind to a human DISC1 protein comprising any of the amino acid substitutions set forth in TABLE 5A. Alternatively, one can also measure an activity of a wild-type or mutant DISC1 protein, such binding or agonist/antagonist activity. Such assays are known in the art. For example, a ligand to the DISC1 protein can be mixed with both wild-type and mutant DISC1 protein to evaluate whether ligand binding of the mutant protein differs from ligand binding to the wild-type protein.

Antibodies directed against wild type or mutant DISC1 polypeptides or allelic variant thereof, which are discussed above, may also be used in disease diagnostics and prognostics. Such diagnostic methods, may be used to detect abnormalities in the level of DISC1 polypeptide expression, or abnormalities in the structure and/or tissue, cellular, or subcellular location of a DISC1 polypeptide. Structural differences may include, for example, differences in the size, electronegativity, or antigenicity of the mutant DISC1 polypeptide relative to the wild-type polypeptide. Protein from the tissue or cell type to be analyzed may easily be detected or isolated using techniques which are well known to one of skill in the art, including but not limited to western blot analysis. For a detailed explanation of methods for carrying out Western blot analysis, see Sambrook et al., 1989, supra, at Chapter 18. The protein detection and isolation methods employed herein may also be such as those described in Harlow and Lane, for example, (Harlow, E. and Lane, D., 1988, “Antibodies: A Laboratory Manual”, Cold Spring Harbor Laboratory Press, Cold Spring Harbor, N.Y.), which is incorporated herein by reference in its entirety.

This can be accomplished, for example, by immunofluorescence techniques employing a fluorescently labeled antibody (see below) coupled with light microscopic, flow cytometric, or fluorimetric detection. The antibodies (or fragments thereof) useful in the present invention may, additionally, be employed histologically, as in immunofluorescence or immunoelectron microscopy, for in situ detection of DISC1 polypeptides. In situ detection may be accomplished by removing a histological specimen from a patient, and applying thereto a labeled antibody of the present invention. The antibody (or fragment) is preferably applied by overlaying the labeled antibody (or fragment) onto a biological sample. Through the use of such a procedure, it is possible to determine not only the presence of the DISC1 polypeptide, but also its distribution in the examined tissue. Using the present invention, one of ordinary skill will readily perceive that any of a wide variety of histological methods (such as staining procedures) can be modified in order to achieve such in situ detection.

Often a solid phase support or carrier is used as a support capable of binding an antigen or an antibody. Well-known supports or carriers include glass, polystyrene, polypropylene, polyethylene, dextran, nylon, amylases, natural and modified celluloses polyacrylamides, gabbros, and magnetite. The nature of the carrier can be either soluble to some extent or insoluble for the purposes of the present invention. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. Preferred supports include polystyrene beads Those skilled in the art will know many other suitable carriers for binding antibody or antigen, or will be able to ascertain the same by use of routine experimentation.

One means for labeling an anti-DISC1 polypeptide specific antibody is via linkage to an enzyme and use in an enzyme immunoassay (EIA) (Voller, “The Enzyme Linked Immunosorbent Assay (ELISA)”, Diagnostic Horizons 2:1-7, 1978, Microbiological Associates Quarterly Publication, Walkersville, Md.; Voller, et al., J. Clin. Pathol. 31:507-520 (1978); Butler, Meth. Enzymol. 73:482-523 (1981); Maggio, (ed.) Enzyme Immunoassay, CRC Press, Boca Raton, Fla., 1980; Ishikawa, et al., (eds.) Enzyme Immunoassay, Kgaku Shoin, Tokyo, 1981). The enzyme which is bound to the antibody will react with an appropriate substrate, preferably a chromogenic substrate, in such a manner as to produce a chemical moiety which can be detected, for example, by spectrophotometric, fluorimetric or by visual means. Enzymes which can be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. The detection can be accomplished by calorimetric methods which employ a chromogenic substrate for the enzyme. Detection may also be accomplished by visual comparison of the extent of enzymatic reaction of a substrate in comparison with similarly prepared standards.

Detection may also be accomplished using any of a variety of other immunoassays. For example, by radioactively labeling the antibodies or antibody fragments, it is possible to detect fingerprint gene wild type or mutant peptides through the use of a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a gamma counter or a scintillation counter or by autoradiography.

It is also possible to label the antibody with a fluorescent compound. When the fluorescently labeled antibody is exposed to light of the proper wave length, its presence can then be detected due to fluorescence. Among the most commonly used fluorescent labeling compounds are fluorescein isothiocyanate, rhodamine, phycoerythrin, phycocyanin, allophycocyanin, o-phthaldehyde and fluorescamine.

The antibody can also be detectably labeled using fluorescence emitting metals such as ¹⁵²Eu, or others of the lanthanide series. These metals can be attached to the antibody using such metal chelating groups as diethylenetriaminepentacetic acid (DTPA) or ethylenediaminetetraacetic acid (EDTA).

The antibody also can be detectably labeled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

Likewise, a bioluminescent compound may be used to label the antibody of the present invention. Bioluminescence is a type of chemiluminescence found in biological systems in, which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

Moreover, it will be understood that any of the above methods for detecting alterations in a gene or gene product or polymorphic variants can be used to monitor the course of treatment or therapy.

If a polymorphic region is located in an exon, either in a coding or non-coding portion of the gene, the identity of the allelic variant can be determined by determining the molecular structure of the mRNA, pre-mRNA, or cDNA. The molecular structure can be determined using any of the above described methods for determining the molecular structure of the genomic DNA, e.g., DHPLC, sequencing and SSCP.

The methods described herein may be performed, for example, by utilizing pre-packaged diagnostic kits, such as those described above, comprising at least one probe or primer nucleic acid described herein, which may be conveniently used, e.g., to determine whether a subject has or is at risk of developing a disease associated with a specific DISC1 or DISC2 allelic variant.

Sample nucleic acid for using in the above-described diagnostic and prognostic methods can be obtained from any cell type or tissue of a subject. For example, a subject's bodily fluid (e.g. blood) can be obtained by known techniques (e.g. venipuncture). Alternatively, nucleic acid tests can be performed on dry samples (e.g. hair or skin). Fetal nucleic acid samples can be obtained from maternal blood as described in International Patent Application No. WO91/07660 to Bianchi. Alternatively, amniocytes or chorionic villi may be obtained for performing prenatal testing.

Diagnostic procedures may also be performed in situ directly upon tissue sections (fixed and/or frozen) of patient tissue obtained from biopsies or resections, such that no nucleic acid purification is necessary. Nucleic acid reagents may be used as probes and/or primers for such in situ procedures (see, for example, Nuovo, G. J., 1992, PCR in situ hybridization: protocols and applications, Raven Press, New York).

In addition to methods which focus primarily on the detection of one nucleic acid sequence, profiles may also be assessed in such detection schemes. Fingerprint profiles may be generated, for example, by utilizing a differential display procedure, Northern analysis and/or RT-PCR.

### Pharmacogenomics

Knowledge of the identity of the allele of one or more DISC1 of DISC2 gene polymorphic regions in an individual (the DISC1 or DISC2 genetic profile), alone or in conjunction with information on other genetic defects contributing to the same disease (the genetic profile of the particular disease) also allows a customization of the therapy for a particular disease to the individual's genetic profile, the goal of “pharmacogenomics”. For example, subjects having a specific allele of a DISC1 or DISC2 gene may or may not exhibit symptoms of a particular disease or be predisposed to developing symptoms of a particular disease. Further, if those subjects are symptomatic, they may or may not respond to a certain drug, e.g., a specific DISC1 or DISC2 therapeutic, such as, e.g., an inhibitor of DISC1 or DISC2 activity or binding, but may respond to another. Thus, generation of a DISC1 or DISC2 genetic profile, (e.g., categorization of alterations in DISC1 or DISC2 genes which are associated with the development of a particular disease), from a population of subjects, who are symptomatic for a disease or condition that is caused by or contributed to by a defective and/or deficient DISC1 or DISC2 gene and/or protein (a DISC1 or DISC2 genetic population profile) and comparison of an individual's DISC1 or DISC2 profile to the population profile, permits the selection or design of drugs that are expected to be safe and efficacious for a particular patient or patient population (i.e., a group of patients having the same genetic alteration).

For example, a DISC1 or DISC2 population profile can be performed by determining the DISC1 or DISC2 profile, e.g., the identity of DISC1 or DISC2 alleles, in a patient population having a disease, which is associated with one or more specific alleles of DISC1 or DISC2 polymorphic regions. Optionally, the DISC1 or DISC2 population profile can further include information relating to the response of the population to a DISC1 or DISC2 therapeutic, using any of a variety of methods, including, monitoring: 1) the severity of symptoms associated with the DISC1 or DISC2 related disease, 2) DISC1 or DISC2 gene expression level, 3) DISC1 or DISC2 mRNA level, 4) DISC1 protein level, 5) eosinophil level, and/or 6) leukotriene level, and dividing or categorizing the population based on particular DISC1 or DISC2 alleles. The DISC1 or DISC2 genetic population profile can also, optionally, indicate those particular DISC1 or DISC2 alleles which are present in patients that are either responsive or non-responsive to a particular therapeutic. This information or population profile, is then useful for predicting which individuals should respond to particular drugs, based on their individual DISC1 or DISC2 profile.

In a preferred embodiment, the DISC1 or DISC2 profile is a transcriptional or expression level profile and step (i) is comprised of determining the expression level of DISC1 proteins, alone or in conjunction with the expression level of other genes known to contribute to the same disease at various stages of the disease.

Pharmacogenomic studies can also be performed using transgenic animals. For example, one can produce transgenic mice, e.g., as described herein, which contain a specific allelic variant of a DISC1 or DISC2 gene. These mice can be created, e.g., by replacing their wild-type DISC1 or DISC2 gene with an allele of the human DISC1 or DISC2 gene. The response of these mice to specific DISC1 or DISC2 therapeutics can then be determined.

### Methods of Treatment

The present invention provides for both prophylactic and therapeutic methods of treating a subject having or likely to develop a disorder associated with specific DISC1 or DISC2 alleles and/or aberrant DISC1 expression or activity, e.g., disorders or diseases associated with aberrant neurological functions, such as neuropsychiatric diseases or disorders.

The DISC1 or DISC2 nucleic acid molecules, polypeptides and antibodies of the present invention may be used, for example, in therapeutic methods to treat disorders, such as neuropsychiatric disorder (including, for example, schizophrenia, schizoaffective disorder, bipolar affective disorder, unipolar affective disorder, attention deficit disorder, and adolescent conduct disorder). In addition, compounds that bind to a DISC1 or DISC2 nucleic acid or polypeptide of the invention, compounds that modulate DISC1 or DISC2 gene expression, and compounds that interfere with or modulate binding of a DISC1 or DISC2 nucleic acid or polypeptide with a binding compound may be useful, e.g., in methods for treating such neuropsychiatric disorders.

For example, in a preferred embodiment, compounds that specifically bind to variant DISC1 or DISC2 nucleic acid of the present invention or, alternatively, compounds that specifically bind to a variant DISC1 or DISC2 gene product encoded by such a nucleic acid molecule may be used to inhibit the expression or activity of that variant DISC1 or DISC2 gene or gene product, while not inhibiting the expression or activity of a wild-type DISC1 or DISC2 gene or its gene product.

Prophylactic Methods. In one aspect, the invention provides a method for preventing in a subject, a disease or condition associated with a specific DISC1 or DISC2 allele and/or an aberrant DISC1 or DISC2 expression or activity, such as a neuropsychiatric disorder, e.g., schizophrenia, and medical conditions resulting therefrom, by administering to the subject an agent which counteracts the unfavorable biological effect of the specific DISC1 or DISC2 allele. Subjects at risk for such a disease can be identified by a diagnostic or prognostic assay, e.g., as described herein. Administration of a prophylactic agent can occur prior to the manifestation of symptoms associated with specific DISC1 or DISC2 alleles, such that a disease or disorder is prevented or, alternatively, delayed in its progression. Depending on the identity of the DISC1 or DISC2 allele in a subject, a compound that counteracts the effect of this allele is administered. The compound can be a compound modulating the activity of a DISC1 polypeptide or DISC2 RNA, e.g., an inhibitor. The treatment can also be a specific diet, or environmental alteration. In particular, the treatment can be undertaken prophylactically, before any other symptoms are present. Such a prophylactic treatment could thus prevent the development of an aberrant neurological function or aberrant neuropsychiatric profile such as those displayed in, e.g., schizophrenia, schizoaffective disorder, bipolar disorder, unipolar affective disorder and adolescent conduct disorder. The prophylactic methods are similar to therapeutic methods of the present invention and are further discussed in the following subsections.

Therapeutic Methods. The invention further provides methods of treating subjects having a disease or disorder associated with a specific allelic variant of a polymorphic region of a DISC1 or DISC2 gene Preferred diseases or disorders include those associated with aberrant neurological function, and disorders resulting therefrom (e.g., neuropsychiatric diseases and disorders, such as, for example, schizophrenia, schizoaffective disorder, bipolar disorder, unipolar affective disorder and adolescent conduct disorder).

In one embodiment, the method comprises (a) determining the identity of the allelic variant; and (b) administering to the subject a compound that compensates for the effect of the specific allelic variant. The polymorphic region can be localized at any location of the gene, e.g., in the promoter (e.g., in a regulatory element of the promoter), in an exon, (e.g., coding region of an exon), in an intron, or at an exon/intron border. Thus, depending on the site of the polymorphism in the DISC1 or DISC2 gene, a subject having a specific variant of the polymorphic region which is associated with a specific disease or condition, can be treated with compounds which specifically compensate for the allelic variant.

In a preferred embodiment, the identity of one or more of the nucleotides of a DISC1 gene identified in TABLE 5 can be determined.

In a particularly preferred embodiment, it is determined that a subject has one or more of the following (WT/SNP): C/T at position 1872; and A/T at position 2163; of SEQ ID NO:1, respectively.

If a subject has one or more of the polymorphisms of the invention (see TABLE 5), that subject can have or be predicted to be at risk for developing a neuropsychatric disorder, e.g. schizophrenia. The neuropsychiatric disorder can be prevented from occurring or can be reduced by administering to the subject a pharmaceutically effective amount of a compound found to inhibit the activity or binding of the DISC1 polypeptide or DISC2 gene product, or modifies the transcription or expression of the DISC1 or DISC2 gene.

Generally, the allelic variant can be a mutant allele, i.e., an allele which when present in one, or preferably two copies, in a subject results in a change in the phenotype of the subject. A mutation can be a substitution, deletion, and/or addition of at least one nucleotide relative to the wild-type allele (i.e., the reference sequence). Depending on where the mutation is located in the DISC1 or DISC2 gene, the subject can be treated to specifically compensate for the mutation. For example, if the mutation is present in the coding region of the DISC1 gene and results in a more active DISC1 protein, the subject can be treated, e.g. by administration to the subject of a DISC1 inhibitor, such that the administration of an _inhibitor prevents aberrant neurological function associated with the DISC1 protein. In addition, wild-type DISC1 protein or nucleic acid coding sequence/cDNA can be administered to compensate for the endogenous mutated form of the DISC1 protein. Likewise, an inhibitor or compensator can be administered to counteract any abberant activity of a DISC2 RNA. Nucleic acids encoding wild-type human DISC1 protein are set forth in SEQ ID NO:2. Furthermore, depending on the site of the mutation in the DISC1 protein and the specific effect on its activity, specific treatments can be designed to compensate for that effect.

Yet in another embodiment, the invention provides methods for treating a subject having a mutated DISC1 or DISC2 gene, in which the mutation is located in a regulatory region of the gene. Such a regulatory region can be localized in the promoter of the gene, in the 5′ or 3′ untranslated region of an exon, or in an intron. A mutation in a regulatory region can result in increased production of DISC1 protein or DISC2 RNA, decreased production of DISC1 protein or DISC2 RNA, or production of DISC1 protein or DISC2 RNA having an aberrant tissue distribution. The effect of a mutation in a regulatory region upon the DISC1 protein or DISC2 RNA can be determined, e.g., by measuring the protein level or mRNA level in cells having DISC1 and DISC2 genes with such mutations and which, normally (i.e., in the absence of the mutation) produce DISC1 protein or DISC2 RNA. The effect of a mutation can also be determined in vitro. For example, if the mutation is in the promoter, a reporter construct can be constructed which comprises the mutated promoter linked to a reporter gene, the construct transfected into cells, and comparison of the level of expression of the reporter gene under the control of the mutated promoter and under the control of a wild-type promoter. Such experiments can also be carried out in mice transgenic for the mutated promoter. If the mutation is located in an intron, the effect of the mutation can be determined, e.g., by producing transgenic animals in which the mutated DISC1 or DISC2 gene has been introduced and in which the wild-type gene may have been knocked out Comparison of the level of expression/transcription of DISC1 or DISC2 in the mice transgenic for the mutant human DISC1 or DISC2 gene with mice transgenic for a wild-type human DISC1 or DISC2 gene will reveal whether the mutation results in increased, decreased synthesis of the corresponding protein and/or aberrant tissue distribution of the protein or RNA. Such analysis could also be performed in cultured cells, in which the human mutant DISC1 or DISC2 gene is introduced and, e.g., replaces the endogenous wild-type gene in the cell. Thus, depending on the effect of the mutation in a regulatory region of a DISC1 or DISC2 gene, a specific treatment can be administered to a subject having such a mutation. Accordingly, if the mutation results in increased DISC1 protein or DISC2 RNA levels, the subject can be treated by administration of a compound which reduces DISC1/DISC2 expression, e.g., by reducing gene expression or translation or a compound which inhibits or reduces the activity of the DISC1 protein.

Furthermore, it is likely that subjects having different allelic variants of a DISC1 or DISC2 polymorphic region will respond differently to therapeutic drugs to treat diseases or conditions, such as those associated with neuropsychiatric disorders.

A correlation between drug responses and specific alleles of DISC1 or DISC2 can be shown, for example, by clinical studies wherein the response to specific drugs of subjects having different allelic variants of a polymorphic region of a DISC1 or DISC2 gene is compared. Such studies can also be performed using animal models, such as mice having various alleles of human DISC1 or DISC2 genes and in which, e.g., the endogenous DISC1 or DISC2 gene has been inactivated such as by a knock-out mutation. Test drugs are then administered to the mice having different human DISC1 or DISC2 alleles and the response of the different mice to a specific compound is compared. Accordingly, the invention provides assays for identifying the drug which will be best suited for treating a specific disease or condition in a subject. For example, it will be possible to select drugs which will be devoid of toxicity, or have the lowest level of toxicity possible for treating a subject having a disease or condition.

Monitoring Clinical Therapies. The ability to target populations expected to show the highest clinical benefit, based on the neurological activity or disease genetic profile, can enable: 1) the repositioning of marketed drugs with disappointing market results; 2) the rescue of drug candidates whose clinical development has been discontinued as a result of safety or efficacy limitations, which are patient subgroup-specific; and 3) an accelerated and less costly development for drug candidates and more optimal drug labeling (e.g., since the use of DISC1 or DISC2 as a marker is useful for optimizing effective dose).

In situations in which the disease associated with a specific DISC1 or DISC2 allele is characterized by an abnormal protein or RNA expression, the treatment of an individual with a DISC1 or DISC2 therapeutic can be monitored by determining DISC1 or DISC2 characteristics, such as DISC1 protein level or activity, DISC1 or DISC2 mRNA levels, and/or transcriptional levels. This measurement will indicate whether the treatment is effective or whether it should be adjusted or optimized. Thus, DISC1 or DISC2 can be used as a marker for the efficacy of a drug during clinical trials.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) comprising the steps of (i) obtaining a preadministration sample from a subject prior to administration of the agent; (ii) detecting the level of expression of a DISC1 protein, DISC1 or DISC2 mRNA, or genomic DNA in the preadministration sample; (iii) obtaining one or more post-administration samples from the subject; (iv) detecting the level of expression or activity of the DISC1 protein, DISC1 or DISC2 RNA, or genomic DNA in the post-administration samples; (v) comparing the level of expression or activity of the DISC1 protein, DISC1/DISC2 RNA, or genomic DNA in the preadministration sample with the DISC1 protein, DISC1 or DISC2 mRNA, or genomic DNA in the post administration sample or samples; and (vi) altering the administration of the agent to the subject accordingly. For example, increased administration of the agent may be desirable to increase the expression or activity of DISC1 or DISC2 to higher levels than detected, i.e., to increase the effectiveness of the agent. Alternatively, decreased administration of the agent may be desirable to decrease expression or activity of DISC1 or DISC2 to lower levels than detected, i.e., to decrease the effectiveness of the agent.

Cells of a subject may also be obtained before and after administration of a DISC1 or DISC2 therapeutic to detect the level of expression of genes other than the DISC1 or DISC2 gene, to verify that the therapeutic does not increase or decrease the expression of genes which could be deleterious. This can be done, e.g., by using the method of transcriptional profiling. Thus, mRNA from cells exposed in vivo to a DISC1 or DISC2 therapeutic and mRNA from the same type of cells that were not exposed to the therapeutic could be reverse transcribed and hybridized to a chip containing DNA from numerous genes, to thereby compare the expression of genes in cells treated and not treated with a DISC1 or DISC2 therapeutic. If, for example a DISC1 or DISC2 therapeutic turns on the expression of a proto-oncogene in an individual, use of this particular therapeutic may be undesirable.

### Other Uses

The identification of different alleles of DISC1 or DISC2 can also be useful for identifying an individual among other individuals from the same species. For example, DNA sequences can be used as a fingerprint for detection of different individuals within the same species (Thompson, J. S. and Thompson, eds., Genetics in Medicine, WB Saunders Co., Philadelphia, Pa. (1991)). This is useful, for example, in forensic studies and paternity testing, as described below.

Forensics Applications. Determination of which specific allele occupies a set of one or more polymorphic sites in an individual identifies a set of polymorphic forms that distinguish the individual from others in the population. See generally National Research Council, The Evaluation of Forensic DNA Evidence (Eds. Pollard et al., National Academy Press, D.C., 1996). The more polymorphic sites that are analyzed, the lower the probability that the set of polymorphic forms in one individual is the same as that in an unrelated individual. Preferably, if multiple sites are analyzed, the sites are unlinked. Thus, the polymorphisms of the invention can be used in conjunction with known polymorphisms in distal genes. Preferred polymorphisms for use in forensics are biallelic because the population frequencies of two polymorphic forms can usually be determined with greater accuracy than those of multiple polymorphic forms at multi-allelic loci.

The capacity to identify a distinguishing or unique set of forensic markers in an individual is useful for forensic analysis. For example, one can determine whether a blood sample from a suspect matches a blood or other tissue sample from a crime scene by determining whether the set of polymorphic forms occupying selected polymorphic sites is the same in the suspect and the sample. If the set of polymorphic markers does not match between a suspect and a sample, it can be concluded (barring experimental error) that the suspect was not the source of the sample. If the set of markers is the same in the sample as in the suspect, one can conclude that the DNA from the suspect is consistent with that found at the crime scene. If frequencies of the polymorphic forms at the loci tested have been determined (e.g., by analysis of a suitable population of individuals), one can perform a statistical analysis to determine the probability that a match of suspect and crime scene sample would occur by chance.

p(ID) is the probability that two random individuals have the same polymorphic or allelic form at a given polymorphic site. For example, in biallelic loci, four genotypes are possible: AA, AB, BA, and BB. If alleles A and B occur in a haploid genome of the organism with frequencies x and y, the probability of each genotype in a diploid organism is (see WO 95/12607):

Homozygote: p(AA)=x²

Homozygote: p(BB)=y²=(1−x)²

Single Heterozygote: p(AB)=p(BA)=xy=x(1−x)

Both Heterozygotes: p(AB+BA)=2xy=2x(1−x)

The probability of identity at one locus (i.e., the probability that two individuals, picked at random from a population will have identical polymorphic forms at a given locus) is given by the equation: p(ID)=(x²).

These calculations can be extended for any number of polymorphic forms at a given locus. For example, the probability of identity p(ID) for a 3-allele system where the alleles have the frequencies in the population of x, y, and z, respectively, is equal to the sum of the squares of the genotype frequencies: P(ID)=x⁴+(2xy)²+(2yz)²+(2xz)²+z⁴+y⁴.

In a locus of n alleles, the appropriate binomial expansion is used to calculate p(ID) and p(exc).

The cumulative probability of identity (cum p(ID)) for each of multiple unlinked loci is determined by multiplying the probabilities provided by each locus: cum p(ID)=p(ID1)p(ID2)p(ID3) . . . p(IDn).

The cumulative probability of non-identity for n loci (i.e., the probability that two random individuals will be difference at 1 or more loci) is given by the equation: cum p(nonID)=1−cum p(ID).

If several polymorphic loci are tested, the cumulative probability of non-identity for random individuals becomes very high (e.g., one billion to one). Such probabilities can be taken into account together with other evidence in determining the guilt or innocence of the suspect.

Paternity Testing. The object of paternity testing is usually to determine whether a male is the father of a child. In most cases, the mother of the child is known, and thus, it is possible to trace the mother's contribution to the child's genotype. Paternity testing investigates whether the part of the child's genotype not attributable to the mother is consistent to that of the puntative father. Paternity testing can be performed by analyzing sets of polymorphisms in the putative father and in the child.

If the set of polymorphisms in the child attributable to the father does not match the set of polymorphisms of the putative father, it can be concluded, barring experimental error, that that putative father is not the real father. If the set of polymorphisms in the child attributable to the father does match the set of polymorphisms of the putative father, a statistical calculation can be performed to determine the probability of a coincidental match.

The probability of parentage exclusion (representing the probability that a random male will have a polymorphic form at a given polymorphic site that makes him incompatible as the father) is given by the equation (see WO 95/12607): p(exc)=xy(1−xy), where x and y are the population frequencies of alleles A and B of a biallelic polymorphic site.

(At a triallelic site p(exc)=xy(1−xy)+yz(1−yz)+xz(1−xz)+3xyz(1−xyz)), where x, y, and z and the respective populations frequencies of alleles A, B, and C).

The probability of non-exclusion is: p(non-exc)=1−p(exc).

The cumulative probability of non-exclusion (representing the values obtained when n loci are is used) is thus:

Cum p(non-exc)=p(non-exc1)p(non-exc2)p(non-exc3) . . . p(non-excn).

The cumulative probability of the exclusion for n loci (representing the probability that a random male will be excluded: cum p(exc)=1−cum p(non-exc).

If several polymorphic loci are included in the analysis, the cumulative probability of exclusion of a random male is very high. This probability can be taken into account in assessing the liability of a putative father whose polymorphic marker set matches the child's polymorphic marker set attributable to his or her father.

Kits. As set forth herein, the invention provides methods, e.g., diagnostic and therapeutic methods, e.g., for determining the type of allelic variant of a polymorphic region present in a DISC1 or DISC2 gene, such as a human DISC1 or DISC2 gene. In preferred embodiments, the methods use probes or primers comprising nucleotide sequences which are complementary to a polymorphic region of a DISC1 or DISC2 gene (e.g., SEQ ID NOS:33-43). Accordingly, the invention provides kits for performing these methods.

In a preferred embodiment, the invention provides a kit for determining whether a subject has or is at risk of developing a disease or condition associated with a specific allelic variant of a DISC1 or DISC2 polymorphic region. In an even more preferred embodiment, the disease or disorder is characterized by an abnormal DISC1 or DISC2 activity. In an even more preferred embodiment, the invention provides a kit for determining whether a subject has or is at risk of developing a neuropsychiatric disease such as, e.g., schizophrenia, schizoaffective disorder, bipolar disorder, unipolar affective disorder and adolescent conduct disorder.

A preferred kit provides reagents for determining whether a subject is likely to develop a neuropsychiatric disease such as, e.g., one of the aforementioned disorders/diseases.

Preferred kits comprise at least one probe or primer which is capable of specifically hybridizing under stringent conditions to a DISC1 or DISC2 sequence or polymorphic region and instructions for use. The kits preferably comprise at least one of the above described nucleic acids. Preferred kits for amplifying at least a portion of a DISC1 or DISC2 gene, e.g., the 5′ promoter region, comprise two primers, at least one of which is capable of hybridizing to an allelic variant sequence. Even more preferred kits comprise a pair of primers selected from the group set forth in TABLE 5 and FIG. 4 (SEQ ID NOS:33-127). The kits of the invention can also comprise one or more control nucleic acids or reference nucleic acids, such as nucleic acids comprising a DISC1 or DISC2 intronic sequence. For example, a kit can comprise primers for amplifying a polymorphic region of a DISC1 or DISC2 gene and a control DNA corresponding to such an amplified DNA and having the nucleotide sequence of a specific allelic variant. Thus, direct comparison can be performed between the DNA amplified from a subject and the DNA having the nucleotide sequence of a specific allelic variant. In one embodiment, the control nucleic acid comprises at least a portion of a DISC1 or DISC2 gene of an individual who does not have a neuropsychiatric disease, aberrant neurological activity, or a disease or disorder associated with an aberrant neurological activity.

Yet other kits of the invention comprise at least one reagent necessary to perform the assay. For example, the kit can comprise an enzyme. Alternatively the kit can comprise a buffer or any other necessary reagent.

The present invention is further illustrated by the following examples which should not be construed as limiting in any way. The contents of all cited references (including, without limitation, literature references, issued patents, published patent applications) as cited throughout this application are hereby expressly incorporated by reference. The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

### 6. EXAMPLES

The invention is also described by means of particular examples. However, the use of such examples anywhere in the specification is illustrative only and in no way limits the scope and meaning of the invention or of any exemplified term. Likewise, the invention is not limited to any particular preferred embodiments described herein. Indeed, many modifications and variations of the invention will be apparent to those skilled in the art upon reading this specification and can be made without departing from its spirit and scope. The invention is therefore to be limited only by the terms of the appended claims along with the full scope of equivalents to which the claims are entitled.

### Example 1

### LOD Analysis of a Region on Human Chromosome 1 Associated With Neuropsychiatric Disorders

This Example describes the identification of a susceptibility locus for schizophrenia and related disorders resides on chromosome 1q32-42, in the region of DISC1 and DISC2.

Evidence for a neuropsychiatric disorder susceptibility locus within the chromosomal region of DISC1 and DISC2 was derived from a linkage analysis of schizophrenia family data. An affected sibling pair linkage analysis was aimed to identify alleles (at polymorphic DNA markers) which were shared by affected siblings within a family (Kruglyak et al., Am J Hum Genet 1996;58:1347-1363). If increased allele sharing was identified across many families, and occurred more often than expected by chance, an inference could be made that a disease susceptibility locus also mapped to the region of increased allele sharing.

### Materials and Methods

Sample population. Nuclear families, with at least one sibling diagnosed with schizophrenia or schizoaffective disorder, and a second sibling with schizophrenia, schizoaffective disorders or a related psychotic disorder, were identified from nationwide registers in Finland (Hovatta et al., Am J Hum Genet 1999;65:1114-24; Ekelund et al., Hum Mol Genet 2000; 12:1049-1057). To facilitate a search for susceptibility loci underlying a narrow, intermediate or broad definition of schizophrenia, families were grouped into three classes. Specifically, prior to genetic analysis, each family was characterized as belonging to one, two, or three of three increasingly more inclusive diagnostic groups. These groups, as well as the number of families meeting the criteria for each class, are detailed in TABLE 1.

TABLE 1
Families for Linkage Analysis of Schizophrenia

| | No. of |
|---|---|
| Type of Family | Families |
| 1: At least two siblings with schizophrenia | 130 |
| 2: At least two siblings with schizophrenia or schizoaffective | 164 |
| disorder | |
| 3: At least two siblings with schizophrenia, schizoaffective | 175 |
| disorder, schizophreniform disorder, delusional disorder, brief | |
| psychotic disorder or psychoses not otherwise specified (NOS) | |

Genotyping. Genomic DNA samples were extracted from whole blood using standard methods (Blin and Stafford, Nucleic Acids Res 1976;3:2303-8). DNAs were quantitated by Hoechst dye fluorescence using a CytoFluor 3000 fluorimeter (PerSeptive Biosystems, Framingham, Mass.), normalized at 40 ng/μl, and stored at 4° C. Microsatellite marker loci, that map to introns of DISC1, were selected from public databases. PCR primers, labeled for analysis on the ABI 377XL DNA sequencer (Applied Biosystems, Foster City, Calif.), were prepared using standard oligonucleotide synthesis chemistry. Each genotype was double-scored, once by an expert human technician and once by a proprietary software package; incongruities between the two were resolved by the human scorer. Finished marker data for each pedigree were checked for Mendelian inheritance; raw data for all observed deviations were reevaluated.

Linkage Analysis. Nonparametric affected sibling pair linkage analysis was carried out using Genehunter v1.0 (Kruglyak et al 1996, supra) as modified by Kong and Cox (Am J Hum Genet 1997;61: 1179-1188). Allele frequencies were determined using actual or imputed founder genotypes. Evidence for linkage was determined from the LOD score and its associated p-value. The LOD score is calculated as the logarithm (base 10) of the odds of observing the data given linkage of the microsattellite marker to a susceptibility locus versus the odds of observing the data under the null hypothesis of no linkage. Thus, a LOD score of 2 is interpreted as 100:1 odds in favor of linkage.

### Results

Microsattellite markers D1S251, D1S3462, and D1S2702, mapping to DISC1 introns 1, 8, and 9, were identified from public resources. The markers were genotyped in the schizophrenia family study DNA and analyzed for linkage to a neuropsychiatric disorders within the family sets defined in TABLE 1. Single marker LOD scores, as well as their corresponding p-values, are presented in TABLE 2. While these LOD scores do not meet criteria for genome wide significance (due to the adjustment to the p-value that must be made given the multiple tests performed across the genome), the LOD scores for D1S3462 and D1S2709 do reach significance for assessing a single candidate region (Lander and Kruglyak, Nat Genet 1996;11:241-7). Thus, this region is judged to be linked to neuropsychiatric disorders and the strength of this linkage is greatest for the broad (Type 3) definition of schizophrenia.

TABLE 2
Single Marker LOD Scores for Markers Mapping to DISC1

| | LOD Scores | | |
|---|---|---|---|
| Marker | Family Type 1 | Family Type 2 | Family Type 3 |

| | | | |
|---|---|---|---|
| D1S251 | 0.29 | 0.21 | 0.16 |
| D1S3462 | 1.67* | 1.39* | 1.31* |
| D1S2709 | 1.88* | 2.69** | 2.88** |

*p < 0.001
**p < 0.005

This Example thereby shows that the genomic sequences comprising DISC1 and DISC2 are related to neuropsychiatric disorders (e.g., schizophrenia). The novel SNPs disclosed in this application are thus ones that can be used to diagnose and treat such disorders in a general population, and not just in a special pedigree.

### Example 2

### Determining the Genomic Structure for DISC1 and DISC2

So that the DISC1 and DISC2 genes could be further analyzed for nucleotide variants and, in particular, for nucleotide variants which correlate with a neuropsychiatric disorder such as schizophrenia, schizoaffective disorder, bipolar affective disorder, unipolar affective disorder and adolescent conduct disorder, the genomic structure of these genes was first determined. Specifically, although the cDNA sequences of both the DISC1 and the DISC2 genes have been previously described (see, in particular, Millar et al., Human Molecular Genetics 2000, 9:1415-1423) their genomic sequences remain unknown, including their intron sequences, the intron-exon boundaries and 5′-promoter sequences of the DISC1 and DISC2 genes. This example describes the elucidation of the genomic sequences and structures for these genes.

Briefly, the genomic structures of DISC1 and DISC2 are determined by comparing the cDNA sequences shown in FIGS. 1A-C (SEQ ID NO:1) and in FIGS. 3A-G (SEQ ID NO:3), respectively for each of these genes to the sequences of large, genomic DNA clones (BACs) that contain genomic sequences for segments of human chromosome 1 which contain DISC1 and DISC2. Such comparisons were done using the Basic Local Alignment Search Tool (BLAST; see, for example, Karlin&Altschul, Proc. Natl. Acad. U.S.A. 1990, 87:2267-2268; Altschul et al., J. Mol. Biol. 1990, 215:403-410; Altschul et al., Nature Genetics 1993, 3:266-272; and Altschul et al., Nucl. Acids. Res. 1997, 25:3389-3402) and Sequencher 3.1.1 (Gene Codes Corporation, Ann Arbor, Mich.). The DISC1 and DISC2 cDNA sequences were compared to the sequences of BACs which are publicly available from the GenBank database, and identified in the following Table by their GenBank Accession number (including the appropriate version number) and their GeneInfo Identification (GI) number. The DISC1 and DISC2 sequence was also compared to a BAC referred to herein as zlhXdisc43 or “disc43”. The nucleotide sequence for this BAC is provided here in SEQ ID NO:4.

TABLE 3
BACs Used to Evaluate the Genomic Structure of DISC1 and DISC2

| BAC | GenBank | |
|---|---|---|
| Clone | Accession No. | GI No./ID |
| RP11-17H4 | AC011655.4 | 7239563 |
| RP11-9801 | AL359543.4 | 8979569 |
| RP4-584N17 | AL136171.10 | 9188229 |
| RP5-865N13 | AL353052.2 | 9213047 |
| RP4-730B13 | AL161743.14 | 9588180 |
| RP4-730B13 | AL161743.15 | 9717048 |

Using BLAST and Sequencher to align the DISC1 and DISC2 cDNA sequences with the above BAC sequences, regions of identity were identified between the transcribed cDNA sequence and the sequences of the genomic region from which they were transcribed. These regions of sequence identity correspond to exon regions of the DISC1 and DISC2 genomic sequences, respectively. The boundaries of the exon regions were confirmed by the presence of known, conserved splice elements flanking these exon sequences.

TABLE 4

Exon/Intron Boundaries of Partial DISC1 Genomic Sequences

| | | | | |
|---|---|---|---|---|
| Exon | GI No. | Residues¹ | Boundary Sequences | SEQ ID NO: |

| | | | | |
|---|---|---|---|---|
| 1 | 7239563 | 133392- | GGAAGGAGCA . . . | 5 and 6 |
| | | 133273 | CACCGCGCAGGTAGGGGAGC | |
| 2 | 8979569 | 26151- | TTCTTCCCAGGCAGCCGGGA . . . | 7 and 8 |
| | | 25168 | ATGGAGGTCAGTGTCTCTTC | |
| 3 | 8979569 | 18020- | ACCAACATAGGTAATATCCT . . . | 9 and 10 |
| | | 17951 | TATGATAAAGGTGAGTTTTA | |
| 4 | 8979569 | 73785- | GGGTTTCCAGCTGAGACGTT . . . | 11 and 12 |
| | | 73635 | CCACTCAGCAGAGAGTACTT | |
| 5 | 8979569 | 135940- | TTGTTTTAAGGGCCAGCGGA . . . | 13 and 14 |
| | | 136069 | GCAGCTACAGGTGAGCAGGT | |
| 6 | disc43- | 168- | TTCTCTACAGAAAGAAATCG . . . | 15 and 16 |
| | 8979569 | 93742 | CCATAAGGAGGTACTGCTGA | |
| 7 | 9188229 | 135913- | ATTCTTCCAGCCTCCAGGAA . . . | 17 and 18 |
| | | 135859 | CACTACTAAGGTAAGTACCT | |
| 8 | 9188229 | 130963- | CTCCCCCTAGGTGTGTATGA . . . | 19 and 20 |
| | | 130861 | GCCATATCAGGTAACTGGCA | |
| 9 | 9188229 | 112742- | CGTGCTGTAGGAAACCATTT . . . | 21 and 22 |
| | | 112554 | ACTGCCTATGGTAGGTAGTG | |
| 10 | 9588180 | 36876- | TTTCCCCCAGAAACAAGTGT . . . | 23 and 24 |
| | | 36936 | AACTGTGCAGGTAAGGATAA | |
| 11 | 9588180 | 65546- | TCTGTCTCAGCTGCAAGTGT . . . | 25 and 26 |
| | | 65744 | CCCTTTGAAGGTATTGGAAG | |
| 11′ | 9588180 | 65546- | TCTGTCTCAGCTGCAAGTGT . . . | 27 and 28 |
| | | 65810 | ACAGAAAGAGGTCTGTCCTT | |
| 12 | 9588180 | 83196- | CTCTCGCCAGGAATCTTACA . . . | 29 and 30² |
| | | 83315 | TCTCATTCATATCCTTTTCA | |
| 13 | 9213047 | 974-5398 | GTGCCCACAGTCTCTCAGGA . . . | 31 and 32 |
| | | | TATCCAAGGC | |

¹ Note non-conserved splice donor sites in introns 4 and 12.
² There is a discrepancy in the sequence between GI No. 9588180 (genomic DNA clone; see TABLE 3) and 8163868 (cDNA clone; SEQ ID NO:1) at residue 83315. A ″T″ at that locus (residue No. 10 of SEQ ID NO:30) was confirmed in all samples while the cDNA clone has a ″G″.

Regions of sequence contained in the BAC sequences but not in the cDNA sequences are considered to be intron sequences (for those sequences which are flanked by exon sequences) of the DISC1 or DISC2 genes, or are sequences which lie outside the transcribed 5′ and 3′ boundaries of these genes, including a region (referred to here as the 5′-promoter region) of about 1 kb upstream of the transcription start site.

### Example 3

### Detection and Identification of Novel DISC1 and DISC2 Sequence Variations Associated with Neuropsychiatric Disorders

This example describes experiments in which genetic sequences from individuals of selected populations, described supra in Example 1, were analyzed and novel DISC1 and/or DISC2 polymorphisms were identified. These DISC1 and DISC2 variants, as well as the altered gene products they encode, are described here for the first time, and represent novel DISC1 and DISC2 sequences that can be used in the methods described supra, e.g., to diagnose and treat neuropsychiatric disorders, such as schizophrenia, schizoaffective disorder, bipolar affective disorder, unipolar affective disorder, adolescent conduct disorder, etc., in the general population.

### Materials and Methods

PCR Amplification. DISC1 and DISC2 genomic sequences were amplified according to standard PCR protocols described supra, using oligonucleotide primers described below and in FIG. 4.

Single Stranded Conformation Polymorphism (SSCP) Analysis. SSCP analysis of genetic samples was carried out according to standard protocols described previously (see, in particular, Orita et al., Proc. Natl. Acad. Sci. U.S.A. 1989, 86:2766-2770) and using polyacrylamide gels that contain either 0% or 10% glycerol. The resolution achieved in a gel analysis of a specific polymorphism can vary somewhat depending on the glycerol concentration in the gel. Thus, in this screening analysis, both 0% and 10% glycerol gels were used.

Denaturing High Performance Liquid Chromatography (DHPLC). Standard DHPLC (Transgenomic, Inc., Omaha Nebraska) protocols previously described by Oefener & Underhill (Am. J. Hum. Genet. 1995, 57:A266) were used to analyze PCR amplified fragments of DISC1 and DISC2 genomic sequences, with modifications to increase throughput. Specifically, genetic samples were pooled, after PCR amplification, by combining two DNA samples per well in a 96 well plate, giving 48 pooled samples.

WAVEmaker software (Transgenomic, Inc.) was used to predict a melting temperature for each PCR amplified DISC1 or DISC2 sequence analyzed, and to calculate an appropriate buffer gradient for mutation detection. The buffers used for DHPLC analysis consisted of: Buffer A (0.1 M TEAA) and Buffer B (0.1 M TEAA and 25% acetonitrile).

DNA Sequencing. DNA samples were sequenced using standard nucleic acid sequencing techniques discussed supra.

### Results

PCR amplification products of the DISC1 and DISC2 genomic sequences that contain exon (including the intron/exon junction), 5′-UTR, 3′-UTR and regulatory (e.g., 5′-promoter) sequences of the DISC1 and/or DISC2 genes were generated from genetic samples obtained from individuals of the populations described in Example 1, supra. The primers used are provided in FIG. 4. The table in FIG. 4 describes primer sequence pairs (columns 3 and 4) for the identification/amplification of DISC1 and/or DISC2 variants, as well as the location (column 5) and length (column 6) of the amplified sequence. The PCR primers were chosen to amplify DISC1 and/or DISC2 sequences from about 150 to about 450 bp in length, which are preferred size ranges for mutation analysis by the SSCP and DHPLC methods described here.

The genetic samples included DNA samples obtained from individuals suffering from a neuropsychiatric disorder (core schizophrenia or schizoaffective disorder), as well as samples from control individuals who were not suffering from and did not exhibit symptoms of a neuropsychiatric disorder.

The PCR amplification products were analyzed for polymorphisms using either SSCP, DHPLC or both. In the case of SSCP analysis, aliquots of PCR products amplified from the genomic DNA samples of appropriate individuals were heat denatured and electrophoresed in polyacrylamide gels, and variant nucleotides were detected by mobility shifts in the gel. Alternatively, DHPLC analysis used reverse-phase ion-pairing chromatography to detect heteroduplexes generated during amplification of PCR fragments from individuals who are heterozygous at a particular nucleotide locus within the amplified fragment. Such heteroduplexes are detected by their differing retention times on the HPLC column.

If variant nucleotides were detected by either SSCP or DHPLC, the remaining PCR product from the select individual(s) was sequenced to confirm and identify the polymorphism.

TABLE 5A, below, summarizes SNPs identified in the DISC1 genomic sequence described in Example 2, supra. In particular, column 3 (under the title “Residue no.”) specifies the nucleotide residue in the DISC1 genomic sequence, represented by the nucleotide sequences in TABLE 3, where each SNP is located. Column 4 (under the title “mutation”) specifies the identity of the SNP. For example, the first SNP recited in the table (i.e., disc01a) is an SNP located at nucleotide residue number 1872 of SEQ ID NO: 1. This nucleotide is a cytosine (C) in the wild-type DISC1 genomic sequence, whereas in the SNP, the nucleotide is mutated to a thymine (T), as indicated by “C/T” in column 4 of TABLE 5A. Column 5 (under the title “Genomic Region”) specifies the region of the genomic sequence (e.g., the specific exon, intron or 5′-promoter region) where the SNP is located. TABLE 5B shows the polymorphic site and flanking sequences for each polymorphism, together with the corresponding genomic sequence (GI or SEQ ID NO.).

TABLE 5A
SNPs Identified in DISC1 Genomic Sequences

| Polymorphism | | Residue | Mutation | Genomic |
|---|---|---|---|---|
| ID | GI No./ID | No. | (WT/SNP) | Region |
| disc01a | 9188229 | 112716 | C/T | Exon 9 |
| disc02a | 9588180 | 37031 | G/A | Intron 10 |
| disc03a | 9588180 | 65613 | A/T | Exon 11 |
| disc08a | 9213047 | 1372 | G/A | Exon 13 |
| disc16a | 9213047 | 3301 | C/T | Exon 13 |
| disc18a | 9213047 | 3794 | A/G | Exon 13 |
| disc21a | 9213047 | 4555 | C/T | Exon 13 |
| disc22a | 9213047 | 4721 | G/T | Exon 13 |
| disc22b | 9213047 | 4832 | G/A | Exon 13 |
| disc31a | 7239563 | 133614 | C/G | 5′-promoter |
| disc43a | zlhdisc43 | 176 | C/T | Exon 6 |
| | (SEQ ID NO: 4) | | | |

TABLE 5B
SNP Sequences

| Polymorphism ID | Sequence | SEQ ID NO: |
|---|---|---|
| disc01a | GGCTAAAGACcTCACCGAGGA | 33 |
| disc02a | AGGCTGGCCAgTTTCTCTAAT | 34 |
| disc03a | GCTTATCCAGaGCCTACAGCT | 35 |
| disc08a | GTCATTCTTGgGAATGTCTTC | 36 |
| disc16a | GTGTTCCCTAcGGCTTTATCT | 37 |
| disc18a | ACCACAAGCCaTAACTCATCT | 38 |
| disc21a | TGTGAGCCACcGCACCCGGCC | 39 |
| disc22a | GCCCTTTACAgTCATATCCTA | 40 |
| disc22b | CTGGTAAAATgTGAAGTAATA | 41 |
| disc31a | CTGAGATCCcGAGACAGGCT | 42 |
| disc43a | AGAAAGAAATcGAAGCTCTCC | 43 |

Many of the SNPs identified are found in exons of the DISC1 or DISC2 genomic sequence. Thus, these SNPs may also generate an altered, transcribed gene product (e.g., an altered mRNA or an altered cDNA derived therefrom). These altered cDNA sequences are specified in TABLE 6, below, with respect to either the DISC1 cDNA sequence (SEQ ID NO:1) or the DISC2 cDNA sequence (SEQ ID NO:3) set forth in FIGS. 1A-C and in FIGS. 3A-G, respectively.

TABLE 6A
SNPs Identified in DISC1 cDNA Sequences

| Polymorphism | | | Mutation |
|---|---|---|---|
| ID | SEQ ID NO. | Residue No. | (WT/SNP) |
| disc01a | 1 | 1872 | C/T |
| disc03a | 1 | 2163 | A/T |
| disc08a | 1 | 2879 | G/A |
| disc16a | 1 | 4816 | C/T |
| disc18a | 1 | 5309 | A/G |
| disc21a | 1 | 6069 | C/T |
| disc22a | 1 | 6236 | G/T |
| disc22b | 1 | 6347 | G/A |
| disc43a | 1 | 1460 | C/T |

Certain SNPs identified in TABLE 6A, above (i.e., disc08a, disc16a, disc18a, disc21a, disc22a and disc22b) are located within an untranslated region (i.e., the 5′-UTR or the 3′-UTR) of the DISC1 or DISC2 cDNA sequence and are not expected, therefore, to affect the amino acid sequence of a polypeptide encoded by the cDNA. However, other SNPs identified in TABLE 6A (i.e., disc01a, disc02a, and disc43a) are located within the coding region of the indicated cDNA sequence and, further, change a codon of that coding sequence to one for a different amino acid residue. The disc43a mutation is silent, i.e., the altered codon translates to the same amino acid as the wild-type codon. However, the cDNA sequences which comprise the disc01a and disc02a SNPs do encode an altered gene product. Specifically, the polypeptides encoded by these SNPs comprise amino acid residue substitutions. The specific amino acid residue substitutions encoded by each of these SNPs are indicated in TABLE 6B, below.

TABLE 6B
Amino Acid Substitutions Encoded by DISC1 SNPs

| | | | Mutation |
|---|---|---|---|
| Polymorphism ID | SEQ ID NO. | Residue No. | (WT/SNP) |
| disc01a | 2 | 607 | Leu/Phe |
| disc03a | 2 | 704 | Ser/Cys |

Thus, for example, a DISC1 nucleic acid containing the SNP disc01a encodes an altered DISC1 polypeptide. Specifically, this SNP encodes a variant of the polypeptide sequence set forth in SEQ ID NO:2 in which the amino acid residue at position 607 of this sequence is a phenylalanine (Phe) rather than a leucine (Leu).

Nucleic acid molecules containing sequences which the SNPs disclosed here, as well as polypeptides encoded by such nucleic acids and containing the amino acid substitutions described above, are novel DISC1 and DISC2 sequences of the present invention. Because these SNPs and amino acid substitutions are ones which correlate with the presence of a neuropsychiatric disorder (for example, schizophrenia, schizoaffective disorder, bipolar affective disorder, unipolar affective disorder and adolescent conduct disorder), these nucleic acids and polypeptides are particularly useful in the methods of the present invention, e.g., to diagnose and treat such disorders.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that all values are approximate, and are provided for description.

Patents, patent applications, publications, product descriptions, and protocols are cited throughout this application, the disclosures of which are incorporated herein by reference in their entireties for all purposes.

What is claimed is:

1. An isolated nucleic acid which comprises a nucleotide sequence of a polymorphic region of a DISC1 allelic variant, wherein the DISC1 allelic variant has a nucleotide sequence that differs from a reference nucleotide sequence selected from the group consisting of:
(a) the nucleotide sequence set forth in SEQ ID NO:1;
(b) a DISC1 nucleotide sequence contained in the clone RP11-17H4, RP11-9801, RP4-584N17, RP5-865N13 or RP4-730B13; and
(c) the nucleotide sequence set forth in SEQ ID NO:4.

2. The isolated nucleic acid of claim 1 wherein the polymorphic region is located in a 5′ promoter region.

3. The isolated nucleic acid of claim 1 wherein the polymorphic region is located in an intron.

4. The isolated nucleic acid of claim 1 wherein the polymorphic region is located in an exon.

5. The isolated nucleic acid of claim 1 which comprises a nucleotide sequence selected from the group consisting of SEQ ID NOS:33-43 and complementary sequences thereof.

6. A kit for amplifying or determining the molecular structure of at least a portion of a DISC1 nucleic acid, which kit comprises:
a probe or primer capable of hybridizing to a polymorphic region of a DISC1 nucleic acid; and
instructions for use.

7. The kit of claim 6, wherein the DISC1 nucleic acid is from a human DISC1 gene.

8. The kit of claim 7 wherein the probe or primer is capable of hybridizing to a polymorphic region of a DISC1 allelic variant, wherein the DISC1 allelic variant has a nucleotide sequence that differs from a reference nucleotide sequence selected from the group consisting of:
(a) the nucleotide sequence set forth in SEQ ID NO:1;
(b) a DISC1 nucleotide sequence contained in the clone RP11-17H4, RP11-9801, RP4-584N17, RP5-865N13 or RP4-730B13;
(c) the nucleotide sequence set forth in SEQ ID NO:4; and
(d) complementary sequences thereof.

9. The kit of claim 8 wherein the polymorphic region is located in a 5′ promoter region.

10. The kit of claim 8 wherein the polymorphic region is located in a 3′ untranslated region.

11. The kit of claim 8 wherein the polymorphic region is located in an intron.

12. The kit of claim 8 wherein the polymorphic region is located in an exon.

13. The kit of claim 8 wherein the polymorphic region comprises a nucleotide sequence selected from the group consisting of SEQ ID NOS:33-43 and complementary sequences thereof.

14. The kit of claim 8 wherein the probe or primer is a single stranded nucleic acid.

15. The kit of claim 8 wherein the probe or primer is labeled.

16. The kit of claim 8 wherein the probe or primer has a nucleotide sequence from about 15 to about 30 nucleotides in length.

17. The kit of claim 16 wherein the probe or primer comprises a nucleotide sequence selected from the group consisting of SEQ ID NOS:33-127 and complementary sequences thereof.

18. A kit according to claim 16 which comprises a first primer and a second primer, wherein the first and second primers are selected from the group consisting of SEQ ID NOS:33-127 and complementary sequences thereof.

19. A kit for determining whether a subject is at risk of developing a neuropsychiatric disorder, which kit comprises:
a probe or primer that is capable of hybridizing to a polymorphic region of a DISC1 nucleic acid; and
instructions for use.

20. The kit of claim 19, wherein the neuropsychiatric disorder is schizophrenia, schizoaffective disorder, bipolar disorder, unipolar affective disorder and adolescent conduct disorder.

21. A method for detecting a DISC1 allelic variant, which method comprises contacting a sample DISC1 nucleic acid with a probe or primer complementary to a polymorphic region of a DISC1 allelic variant so that the DISC1 allelic variant is detected in the sample DISC1 nucleic acid.

22. The method of claim 21 wherein the DISC1 allelic variant has a nucleotide sequence that differs from a reference nucleotide sequence selected from the group consisting of:
(a) the nucleotide sequence set forth in SEQ ID NO:1;
(b) a DISC1 nucleotide sequence contained in the clone RP1-17H4, RP11-9801, RP4-584N17, RP5-865N13 or RP4-730B13;
(c) the nucleotide sequence set forth in SEQ ID NO:4; and
(d) complementary sequences thereof.

23. The method of claim 21 further comprising determining the identity of the DISC1 allelic variant.

24. A method according to claim 23 which comprises determining the identity of at least one nucleotide of the sample DISC1 nucleic acid.

25. The method of claim 24 wherein the sequence of the polymorphic region of the sample DISC1 nucleic acid is determined.

26. The method of claim 23 wherein the identity of the DISC1 allelic variant is determined by restriction enzyme analysis.

27. The method of claim 23 wherein the identity of the DISC1 allelic variant is determined by single-stranded conformational polymorphism.

28. The method of claim 23 wherein the identity of the DISC1 allelic variant is determined by allelic specific hybridization.

29. The method of claim 21 wherein the identity of the DISC1 allelic variant is determined by primer specific extension.

30. The method of claim 21 wherein the identity of the DISC1 allelic variant is determined by an oligonucleotide ligation assay.

31. The method of claim 21 wherein the DISC1 allelic variant is an allelic variant of a human DISC1 gene.

32. The method of claim 21 wherein the nucleotide sequence of the probe or primer is from about 15 to about 30 nucleotides in length.

33. The method of claim 32 wherein the probe or primer comprises a nucleotide sequence selected from the group consisting of SEQ ID NOS: 33-127 and complementary sequences thereof.

34. A method according to claim 21 which further comprises contacting the sample DISC1 nucleic acid with a second probe or primer, wherein each probe or primer has a nucleotide sequence selected from the group consisting of SEQ ID NOS: 33-127 and complementary sequences thereof.

35. The method of claim 34 which comprises hybridizing the two probes or primers to the sample DISC1 nucleic acid.

36. The method of claim 21 wherein the probe or primer is a single stranded nucleic acid.

37. The method of claim 21 wherein the probe or primer is labeled.

38. A method for determining whether a subject has or is at risk of developing a disease or disorder associated with a specific DISC1 allelic variant, which method comprises identifying, according to the method of claim 21, the DISC1 allelic variant in a nucleic acid sample from the subject.

39. The method of claim 38 wherein the disease or disorder is a neuropsychiatric disorder.

40. The method of claim 39 wherein the neuropsychiatric disorder is selected from the group consisting of schizophrenia, schizoaffective disorder, bipolar disorder, unipolar affective disorder and adolescent conduct disorder.

41. The method of claim 40 wherein the neuropsychiatric disorder is schizophrenia.

42. A method for selecting an appropriate drug for administration to an individual, which method comprises determining the molecular structure of at least a portion of the DISC1 gene of the individual.

43. The method of claim 42 wherein the molecular structure is determined according to a method that comprises determining the identity of an allelic variant of at least one polymorphic region of the DISC1 gene of the individual.

44. A method for treating a subject having a disease or disorder associated with a specific allelic variant of a polymorphic region of a DISC1 gene, which method comprises:
(a) determining the identity of the allelic variant; and
(b) administering, to the subject, a compound that compensates for the effect of the specific allelic variant.

45. The method of claim 44 wherein the compound is a DISC1 protein activity inhibitor.

46. The method of claim 44 wherein the polymorphic region is located in an exon.

47. The method of claim 44 wherein the polymorphic region is located in an intron.

48. The method of claim 44 wherein the specific allelic variant is a mutant allele.

49. The method of claim 44 wherein the polymorphic region is located in a promoter region.

50. The method of claim 44 wherein the sequence of the specific allelic variant comprises a nucleotide sequence selected from the group consisting of SEQ ID NOS: 33-43 and complementary sequences thereof.

51. The method of claim 44 wherein the specific allelic variant is associated with a neuropsychiatric disorder.

52. The method of claim 51 wherein the neuropsychiatric disorder is selected from the group consisting of schizophrenia, schizoaffective disorder, bipolar disorder, unipolar affective disorder and adolescent conduct disorder.

53. The method of claim 44 wherein the compound modulates DISC1 protein activity levels.


SEQUENCE LISTING (127 sequences)

SEQ ID NO: 1 - DNA, 6930 bp, Homo sapiens
ggaaggagcaggaggcagcccaggcggagcgggaggagctggcagcggggcgcatgccaggcgggggtcctcagggcgccccagccgccgccggcggcggcggcgtgagccaccgcgcaggcagccgggattgcttaccacctgcagcgtgctttcggaggcggcggctggcacggaggccgggctacatgagaagctcgacagggcctgggatcgggttcctttccccagcagtgggcacactgttccggttcccaggaggggtgtctggcgaggagtcccaccactcggagtccagggccagacagtgtggccttgactcgagaggcctcttggtccggagccctgtttccaagagtgcagcagcccctactgtgacctctgtgagaggaacctcggcgcactttgggattcagctcagaggtggcaccagattgcctgacaggcttagctggccgtgtggccctgggagtgctgggtggcagcaagagtttgcagccatggatagttctgagaccctggacgccagctgggaggcagcctgcagcgatggagcaaggcgtgtccgggcagcaggctctctgccatcagcagagttgagtagcaacagctgcagccctggctgtggccctgaggtccccccaacccctcctggctctcacagtgcctttacctcaagctttagctttattcggctctcgcttggctctgccggggaacgtggagaagcagaaggctgcccaccatccagagaggctgagtcccattgccagagcccccaggagatgggagccaaagctgccagcttggacgggcctcacgaggacccgcgatgtctctctcagcccttcagtctcttggctacacgggtctctgcagacttggcccaggccgcaaggaacagctccaggccagagcgtgacatgcattctttaccagacatggaccctggctcctccagttctctggatccctcactggctggctgtggtggtgatgggagcagcggctcaggggatgcccactcttgggacaccctgctcaggaaatgggagccagtgctgcgggactgcctgctgagaaaccggaggcagatggaggtaatatccttaagattaaaacttcagaaacttcaggaagatgcagttgagaatgatgattatgataaagctgagacgttacaacaaagattagaagacctggaacaagagaaaatcagcctgcactttcaacttccttcaaggcagccagctcttagcagtttcctgggtcacctggcagcacaagtccaggctgccttgcgccgtggggccactcagcaggccagcggagatgacacccacaccccactgagaatggagccgaggctgttggaacccactgctcaggacagcttgcacgtgtccatcacgagacgagactggcttcttcaggaaaagcagcagctacagaaagaaatcgaagctctccaagcaaggatgtttgtgctggaagccaaagatcaacagctgagaagggaaatagaggagcaagagcagcaactccagtggcagggctgcgacctgaccccactggtgggccagctgtccctgggtcagctgcaggaggtcagcaaggccttgcaggacaccctggcctcagccggtcagattcccttccatgcagagccaccggaaaccataaggagcctccaggaaagaataaaatccctcaacttgtcacttaaagaaatcactactaaggtgtgtatgagtgagaaattctgcagcaccctgaggaagaaagttaacgatattgaaacccaactaccagccttgcttgaagccaaaatgcatgccatatcaggaaaccatttctggacggctaaagacctcaccgaggagattagatcattaacatcagagagagaagggctggagggactcctcagcaagctgttggtgttgagttccaggaatgtcaaaaagctgggaagtgttaaagaagattacaacagactgagaagagaagtggagcaccaggagactgcctatgaaacaagtgtgaaggaaaatactatgaagtacatggaaacacttaagaataaactgtgcagctgcaagtgtccactgcttgggaaagtgtgggaagctgacttggaagcttgtcgattgcttatccagtgcctacagctccaggaagccaggggaagcctgtctgtagaagatgagaggcagatggatgacttagagggagctgctcctcctattccccccaggctccactccgaggataaaaggaagacccctttgaaggtattggaagaatggaagactcacctcatcccctctctgcactgtgctggaggtgaacagaaagaggaatcttacatcctttctgcagaacttggagaaaagtgtgaagacataggcaagaagctattgtacttggaagatcaacttcacacagcaatccacagtcatgatgaagatctcattcagtctctcaggagggagctccagatggtgaaggaaactctgcaggccatgatcctgcagctccagccagcaaaggaggcgggagaaagagaagctgcagcttcctgcatgacagctggtgtccacgaagcacaagcctgaggagtgacgggatgggggagggaggtgggccaccatgtttggacccggggggctgctcttccctcccccgccatagctaagatgcctgaatcaattacggagatacagagccttgaggtctttcagtggaaaggtggttcatgttcattctcatcagtgtgaaactgaggagtctgcaatttggaatatggagagagagactgatttgctgaatttccttctaaatgtcactcaaaaatttcttttccatgtcattcttgggaatgtcttccacaggatttgagaatagtttcatctcagcccccattagagagaagttggggtgaattctggaaaaatgtctctttttcctgtgccatttgccttctgctgcaacgaaaatatttcctgattcaagattctataaaaaggaaaccaagcataagactctgtcatcatacctgttacacgttcctacaggtgcacaatctaagagagctaattaacctcagagtctggagttaacagcttttcaccttacttctcctgtgatctaatattatcttagaaaaattaatatgcaatttccaaaagatattttggtaagacaacaacctcccagtgatatgccacctttcaattttccttttgtggcaatgattgcatctgaagaaaggatccctgagagtctctgtttcatcaggacattctgaaatttacccacagtgaggctgtggatggatcaggggacctgtataaaatgtttgagcctgttccattttcccgtggaacctgtttcactcaatgccaggcagtgcagcatttaggaaagcagtgcagtactcagtaaggcagtgcagtactcagtaacacaatacagtactcaggcagtgcagtactcagtaagacagtgcagtgctcagtaaggcagtgcaatactcagtaacagtgtagtactcagtaacagtgaagtactcagtaatacagtacagtattcagtaaggcagtgaagtactcagtaatacaatacagtactcagttaggcagtgcagtactcaggaatgcagcacagtactcaggcagtgcaatactcagtgcggtactcagtaacacagtgcagtactcagtaacagtgcagtactcagtaacagtgcagtactcagtaaggcagtgcagtactcagtaacacagtgcagtactcagtaatacagtacagtactcagtaaggcagtatggtactcagtaaagcaatgcaatgctcagtaacacagtgcagtgctcaataaggcagtgcagtgctcagtaaggcagtgaattgcttagtaacacagtgtagtgctcagtaggacagcatagtactcagtaacacagggcagctagtactcagtactataagtactgagtacttatataggcaatgtagtactcagtaaatcagtgcagtactcagtaatgcaagggcatttcaggctcctgctgggctgcttctttggcccagctgggactcctattgagacagctgcaaaacaggctgatttcaattaggcagcacttcccaaagtgcactgaggaaggtggccccaagagaagctctctaaacaaaggagtaccctctctggtcaagtacctttggtaaatacaccataccataatatctgcttggagaaccacaatgcacattagcatattagtctgagagagaacttatagtaaggaaactcacttgattttatctaacctcaaactttccaagtttaatggatcgtgaatttttttcatgtaactcctattcatatcccatagatctagtattgtacagcactgcattctctgaggaagtcccagtccaaactctgatttacatcactttagaaaccacactcacacttttgcagagtgttgagcttaataactacctgccacagattggtaaatttaatccagtggttgttctgtttgtgcttctgttctcatttatgtgtttagggatagtgaggttcctgccttcactaggatccacggatatgagaccatttttgtcatttcctgaagtcacactggcgtttccagaaggcatctggtgctttgctcagccttccatgctgtgcagcacttctgtcctcagtcaaggagatggccatgcttaagccagcaattggctggggtccaggaaacaaagcaaaagcacaatatgtgaatgtgctgattgtgttccctatggctttatctcgagcaaaatacactctacatattttaataataagtataattagcttgttcctggacttcattttcaatgatgaaccaaattcctgaattatttataattgtgtctaaagaaaattatgaactggtcacatggcacttggaatccttgagttaattccagtgaagcaaaacttgggaagagtcaggattggccacattgccaataacaaattcctacttcgacatatgtcttttcaaaaagcctcccagacacaagacatcttaaccgtcactagcccaagtgttttgtattactcagacaccatcatgaaataattctgtgaggtcatgatgtatttgaaaattctgcaagttaataactgccttgaattgtttgaacccgaaataagggttctttggtacctctagtagatagtgtgttcatttccctgctgcaaattttgaagtatttgggcaggtgagtcatgttttaaccacaagccataactcatctgttgtctttgcttggtcttagagtatcattcagaaagtccgctaagggccagcgtgcttcttctggctacacaaccttctcaggacaagcccactgtcttaagccactttgaccctgggagacacaggactgtgtatcctcaatcatactatacagcagtttttgtcaggggaacataaaaatatccaagagaggttagggcttagatttaaaagcatcaaaacaacaacaatggaaatttatgttggcgatagccaagaccacaagcaaaagcacatactggaaatgatgagttagaatctgatttgactgggatgttttatgagaatgtaagtgtgatattatactgtctgccttgctggaatgctggctttcaaatggtcacccatttttctttcactggcctgagttaggacatgctatcagtaatagtcccagttccatccaactttctgaaatttcatttttttttttgagatggagtctctctctgtcacccaagttggagtgcagtggccccgcaatctcggctcactgcaacctctacctcccaggttcaagctattctactgcctcagcctcccaagtagctggggttacaggcatttgccaccgggccctgatgatttttgtatttttagtagagacagggcttcaccatgttggctaggagggtctcaaactcctgacctcaggcgatccacccccacctcggcttcccaaagtgctgagattacaggtgtgagccaccgcacccggccaactttctgaaatttcaaaactgaattgatccttctccaaattagtatatactattggaaacttgtctttccctgcagtaaggctggtttccccaccccagaaacatgtaacggttggtaccatgctaagcccttgccatgctaagccctttacagtcatatcctataatccccatatcaaccttataaggaaggtgtttgtagatgatgcaactgagccttaagaggactaattccctttttctaaggcacagagctggtaaaatgtgaagtaatagtgaacctaacagtcagagacaggcagcatgctcttaactagtgctcttcctaaagttcctttaatgtccttttgagattttgagccatggaacttacttgttcacctggctaagaactcatggccactgtggaaatcttggttagggagtcaaagaaactgagcctggggcaaacgaggcttcccacactgccaggggagcctcactgtgaagtctaggctcagacaggcatcaacaaacctattcaccccaccatcatcctgatctaaccattccccagtcatcccaggaaaaccactcacagcctgacactgggctgactttcttgaagatcctcatccaattggtgtttttcagaagtgttccaatattatgaattctgtgttgtggagaaaagcaaccatgcatttactggtcaatgccttcttgtatatgtaattcaatacttttacttttaatatcctcaccttatctaatctttgaattttgtcatgtaatttattgcttcattaaggttactttttgttatacaaaataaaagctgatatccaaggcaaaaaaaaaaaaaaaaa

SEQ ID NO: 2 - protein, 854 aa, Homo sapiens
MPGGGPQGAPAAAGGGGVSHRAGSRDCLPPAACFRRRRLARRPGYMRSSTGPGIGFLSPAVGTLFRFPGGVSGEESHHSESRARQCGLDSRGLLVRSPVSKSAAAPTVTSVRGTSAHFGIQLRGGTRLPDRLSWPCGPGSAGWQQEFAAMDSSETLDASWEAACSDGARRVRAAGSLPSAELSSNSCSPGCGPEVPPTPPGSHSAFTSSFSFIRLSLGSAGERGEAEGCPPSREAESHCQSPQEMGAKAASLDGPHEDPRCLSQPFSLLATRVSADLAQAARNSSRPERDMHSLPDMDPGSSSSLDPSLAGCGGDGSSGSGDAHSWDTLLRKWEPVLRDCLLRNRRQMEVISLRLKLQKLQEDAVENDDYDKAETLQQRLEDLEQEKISLHFQLPSRQPALSSFLGHLAAQVQAALRRGATQQASGDDTHTPLRMEPRLLEPTAQDSLHVSITRRDWLLQEKQQLQKEIEALQARMFVLEAKDQQLRREIEEQEQQLQWQGCDLTPLVGQLSLGQLQEVSKALQDTLASAGQIPFHAEPPETIRSLQERIKSLNLSLKEITTKVCMSEKFCSTLRKKVNDIETQLPALLEAKMHAISGNHFWTAKDLTEEIRSLTSEREGLEGLLSKLLVLSSRNVKKLGSVKEDYNRLRREVEHQETAYETSVKENTMKYMETLKNKLCSCKCPLLGKVWEADLEACRLLIQCLQLQEARGSLSVEDERQMDDLEGAAPPIPPRLHSEDKRKTPLKVLEEWKTHLIPSLHCAGGEQKEESYILSAELGEKCEDIGKKLLYLEDQLHTAIHSHDEDLIQSLRRELQMVKETLQAMILQLQPAKEAGEREAAASCMTAGVHEAQA

SEQ ID NO: 3 - DNA, 15002 bp, Homo sapiens
  unsure (12055)..(12055): n is a or g or c or t/u, unknown, or other
ttgtggaaaggatggatgggtgggtggatggatggatggatggatggctggatggatggatagaaatgcaacttcaagatctggttctacagtattacatatgaagcaactgagactcagataaaattcagtgagttgctgaagactattcactcattgagcctcacattctgatgctatatatatcaggtgctatgcagatgattagggaagggcaggactgagattaaactgacaccccctacatcctaatccaggctttttccactgtatggcgatgcctcccaaaagaattatcacagcgacttaataaaggagggagaaagttagttgcaaatttcaagcttagaaattcacaggcaaaaatggctgaagggaaataatcacactaatagtgatgcaaggatgtaactggaaaggaaaaaatggagagaaattaaaaacacaaacagatttagtcagggtcctgataaagtgataatgtaatcaataaggcaaatcaaagataaatgattagagatttaaaacttcagcctatcagaaaaacctgagggaaaagattttgaattttttttatttcttccaaaagatcctatttgacatatttttttaaatctaaatgaccaaagatttcgaatttggaaaggcagacacatatctatttcccatataaacatctacatttcttgccacatagcaccgcaacactcatctggttccaaagcagacggcttggccctctgttatcaagagcactatcaccgttctggcccggagagctaagttttcttatacataggtcgtagccaaagctcagaggaaagaagtgacatttggtgagtgtacaggatatgttctccagccactttttaggtgctaagttacatcttgaaataatgtcagaaattggaagatgccgtatttaaacttcactagcccaagccctaattcacctcgacagtttaatatcttaatgtttcttaaaatttgaggactcttagaatgacccctttaaaaatctgctgaaaaactggatctttttgtgggaaaaactgtccatacctacatatctaaaatgtgtatgtggtttcagtctctcctgtgcacagtccctcctgctttgaagggatccccaacatgtttttttcctagaggtagttggtcacaaccatcggataacatgcttatcacctgggtgataaaatattctggacaccaaaccccgatgacatgcaatttacctatataaagaacctgcacatgtattcctgaacctaaaataaaaagttgtaaaaaataataatacaagtaaaagagctttctgcatgtgttataaatctgttcaggaggactttccactatgctttcaagagaagtgtcttgaagaatttagcatcactctgaattcccaaaaagctctgtgcttttcaaggtgcttttgaggaagaaggatgatgcttcaactgtcttctttgctgtgacttctaggaagttcattatcacatcatattctcaaccaaataattaacacttgacattggcattttcttctgtctttgtttttaaattttattgttgtttgctaattttattgtttttatctttgttgtatgtattgtcccggctctccatttcctccctctccctgccaataaaagagacccatagattttattcttttgttctactttttcatcaatgtttcttctacatatatctgacatatatggaaaaaagaattactgcctatatatgcagaacttgtaactcacaggatttctaaactaaaagacttttcttaataggtgtgaagtgaaattctgcaagtagctgcatctgggaacttcagagttaaaccgatatctcagtataatctgtcactgctatagtttcttttctttgacctccaaaccttatgttgaaatttgacccccaatgttgaaggtagagcctaatgggaggtgtctgggtctgggggcagatccctcaagaatagcttggtcatcaatgagtgagttctcactctaatagtttctgggagagctggttgttaaaaagagtgtgtcatgtcccttctctctctttttgcttcctctctcaccagtgatctgcctaggccagctccccttcatcttctgccatgagtggaagcagcctgaggccctcaccagaagcagatgttggcactatgcttcttagacagcctgcaaaactgtgagccaaagaattcccttttctttataaatgacccagcctcaggtattcctctatagcaacgcagatggactaagacactcatgcttcctttcattagctatttcaagctcaaaaattgtgagactgtgccaagaattagggcaggatcttgtcattgagaacttacaacttttctatctaaagcaaaaggtcctaatcccaaaccataattggaaaggtgcctctactccaacctcattgcccttcaactgccccttctctgcctcccattcccctcttactcaccgaaagacaaatgagtttaggctgggcacagttagccatcgttctcagaaaaacaaaatcttgctgcttggtattctcacttagaatcccttcacttagcttatggatcaccctacacagattcctgacagctcactgggccatggaaccagccagatttttgagctacatctgcagtatcttttctactcaggcagctggcataaggtgggaaaattcattaacatgctaatgatgtcctatttaaaattctagtcctgattattctgtgattaaatctctccccttgacgtcggtttcctttatagctgagttacagtgcgaaatcaacaatattaataaagtacaatacagattaccaatttatacacaagggatggtgaaaaactgctgcgtaaatctgagggctgtccttttaacttgaaaagcagttgtaggccgggcgcggtggctcacgcttgtaattccagcactttgggaggcctaagcaggtggatcacctgaggtcaggagtttgagaccagcctggccaacatggtgaaaccccgtcactactaaaaaaatataaaaattagctggcctataatcccagctacctgggaggctgaggcaggagaatcgcttgaacctgggagacggaggttgcagtgagcagagttcgcgccattacacttcaggctggttgcaagaagcaaaacttttgtctcaaaaagagaatgtaaaagcagatgtagcaaacttgtaaaagttgctgatttttttcattccgcccttctttaaataaacacacacattggaatccacaataattaaatgtttatagttaatttccataaatattcaaggcttggaagatagatttagcttttcattctatttcactgcacttaacattgctccgaattcaaatgtgttatcctgaagtctcaagaacagctgacaggagttaaagcacacttgtttatcatctctttatagagattgtgtcttggcaacaaaagccaatggattcggtgatgaggtctgtctccctttgcccttgagtaccttggcatatttcagttccttttctatcaccgtattgttctattactgacactccaaactgaatatccaactcagacaactcaagcaccttccaagaaagacaaacttatcactaccctcattaatttggaaaatgaataaagaagcagaagattcaaggtccagatctgcaatctaaaatgcaggaagagaggactcattcatgtgtcattttaacatctggccccagtaaatgaacaaatattgatggtatagtatatttactattttaaaagacatcatgctggacaccaaagcagtacagtgaaacctaagaacaatgttctgacctcggtggactcacaatctagccagagacacaagatacaatgatattttaagtgatgcatgcagcaaattcatctcaggtgcatcattagagatacacttcacatcattaaaaggtctctgtacgagagacactgctaattgttccctaagactgcaaatctccccttttgctatggaaacagaattcatcactttagtggggcacattgctgcccagggtaaggacagctcccagcccctcttgcaagtagctgttaccatataacaaggttaaggtcacagggatgtggccgtagcagagatgtgtgcagatttctaaaagcgtacttaaaggggatagatgagctctcttccttctttgttttttttctgactggacttggacatgcatgtggctgggtcagccaggttggactgaatggcacatgttgtggggcagagaagaacaagatagaagagtctgggttcctgacaagcagggtggcttgatcatccatgagttgcctttttcccctgaacgctgcattccacaactattgttggaaatcaaaatctttttttaagaatactacatattgctcatttcttaaatggtttcattctctaaggctaaaaagagtcatggtacatgcatgcatatgtgtatgtgcataagtacactctcctttgtactttctctacatttaaacttgaacacagagctccctctgaatttttaaccctcaaaacacaaaaaaagctgaatatggactttgccccagttttatcacattttaataaataaaagcttcaatgtattcaggaattcattatttatttgtttcccacactctcaaccctagattattaatttggttcacctaaagccaactcacctcaaattggttaaaaaaaattaagcacgtgtaaatcaatgtatagggaaagggtggctgatccagcaaaatccaagtgtaaaaacataaagagaggcttcacaacagtaatgagccatctgcaatttagaatcatccatggtaacaatctctcattccatttgtttatgttttattcatcattgtgaaaatacttagtgaagtgacaatgatgagattcaaaaatactgcatggttctttttttaaaaaggatattaatggctttcagggactcaactattcaaatgaaaatattgctggactaaatttgctcaaatttgttcacttcctcttcattcttggatataatatattcttcattttatgacaaaaaactatgtggtgaaaacatgagaaattccgtagtttccatcgtttatacatttaaaaaatcaatagcagaaacctatttcaaatgtacaatcctcattcctatcatgacagagtaaaccaggctgcggtgtcttgggtgagcgtgttgtcattccctagtactacagttatttagcctcaaacgtactgtctctagttacatatatggcaaaaacaaggcttgacctttaagaatgaaaagaaaaaaagataagtactgagctgttaagccaacttaagggtattcattacaaagaagacagaataactactgaacatactctttgattctaatatctctatgaagaaacctctctctacacaaattgatagacattttaaaagaaaatttattcatcccaaattgatgcacttaacttcagctagctgattttatatttctcaatctagcttataaatactactcttcatttacttcaatttacggagtatttacaaaagccctatttcaaatctggttcctatcagccccgttttttcttatttcttcttaaataagcatactttgtaaaatatagccatttattcatatatatatttctttcatttgttttagtaaatgacatttatttaggaaccattttttaagaaataagtttttaccatctcaatttgcactgaaccagcgtcttctgagaccatccctctgggtgcaaatctcagagtaagctcagttactcctaacgactggaaaaggaaagaacacaggaacacttgaactcctctgacaagcagacactttggctaaggcggcattcacatcttctacatcatccttcctggggaaagaagctctcagaaaagtccacacatagaagagccactttatggtcttccctcaattttctcagatcagcctgatttagtctatgattgtgcasacaaggagacggagccaagggacaaaaggaagataattgtcacacaaagaaaatgctcaagaccttgccaagggagagactgctctaaataataacaaggctattagcaaacacaggttgagacagggaacagaaaaagaaaagatgtcwcatgtgaatatgaaaacaaaagctgacaagtwattccattcagtttgccaaggcacctgccaaaaatgtccccaagggaaaagggccatgtgctccatgacgctcttcacagtgaacgttcacatwaaggcacagaacattctgcctggccacaaaacacaacaaatatcatcaatatcttgccggggtaacagttgtgcactacctaccataggcagtctcctggtgctccacttctcttctcagtctgttgtaatcttctttaacacttcccagctttttgacattcctggaactcaacaccaacagcttgctgaggagtccctccagcccttctctctctgatgttaatgatctaatctcctcggtgaggtctttagccgtccagaaatggtttcctacagcacgttgtgggagagaagaagggaaaacaagctttacagcatccacacacatggaagatctagcagcagctaatgtacattcacagcatgggcaaagaaactgcactttgagattatgtwcaatttccttagcagtcctctggggaaagaacctggaaaaatattgctccccttaaactggaaaaatcaccgtcttttttatcaccttattcttgttatgtccattttaggagttccattggtgaagccatctcccgtaaagtatgatgacccaaaaatgtcccagtggaatccagagaaaccacaatgtgtcactccattccaataatatacaaatgtcaaagagatgacatattatagcagtgtggaaggggactgggccatataatcagagttgacctaaattaaattcctcccctgttactcaaattgtgtgacctggagcaagaaaattagcacatctggggcaaatttcttataaaatataattgcaataacacctgactcataggtagttacaagaataaacaagaaattctgtaaagcactgagaaaaatatatgataaattaaatgtagcaagattgcaggatacaaggtcaatgtacaaacatcaatcttatgtctaaataacaaaaaactagaagatgattttttaaaaaatacaatttacaaaatattttaaatctcataatactgagggatactttctttaaatatgtgccagagctctacagtgaaaactatttttccaaaactgctgaaacaaattaaagacaaataatttaaaagatacactgtatacatacactggaagttcaatattggtaagatttcaattttccccacattgattcattgatttaatataattccaatcaaaatgttatgaggttcttttgtagaaatttatgtgcttattctaaaacgtctaaaagtgagctgggcatgggtggctcacacctgtaatcccagcattttgggaggccgaggtggtgggtcccttgaggtcaggtgttcaagaccagcctggccaacatggcgaaaccctatctttactaaaaatataaaaattaatctggcacagtggtaagcacctgtaatcccagctactcaagaggctgaggcaggagaatcacttaaacccaggaggtgtaggttgcagcgagctgagatcctgccactgcactccagcttgagcaacagagtgagacttcatatctaaacaaataaaatctagaagaatgagcccatgagtttgaggctgtagtgcactatgattgcacttttgaacaggcactacactcttgtctgggcaacatagcaagtccccacctcaaagaaggagagggagaaagaagaagaaggaggaggaggaggtgaaagagggagggagggaaggaaggagaagacgaagaagaatgaagaagaataggaagaaagaagaagaaggaagaggaggaggaggatgacccaaamcaatccttaaaaagtgagtcttggtttcaagactaatgctaaagccactataaatcaaggtaatatggcattgaatatacatagacaaacagatcgatggaatagaataaacagtccaggagtggattaacacatatacagccaaccaaagtgccaaagcaagtcaatgaggaaaagaaagtattttcaataaatggtgctggaacaactagctatctacatggaaaaaatattaaccttgattcatacctcaaaccacacaaaaatttaattaaaggtggatcacaggcctacatgtaaaagttaaaactataaagcttctacaaaaaaaaaatgaatatttgattgactttggagtaggcaagaacttcagataagatacagaaaacactattttaaaaaggataaatgggctttatcaaaattaaaaacttctcatcaaaagacacaactaaaaaatgagacgacaagtcacagacttggagaaaatcattgcattatgtttaactgacaaggaattgaatactacaacttgacaattaaaacacaaatggtgccattgaaaaatggtcaaatgacttaacaggcattttacaaagtagacgcacaaatggctaataagcatatgaaaaaacatttgacatcattaatcattaagaaaagaaaaattaaaaccataaatattccactacaagcccactcaaatgtttcaaattaactcaactaaaaatgtcaacattcacaagggtatggagcaactggaactctcacgcatgtaaaatggtcccttcaagaaaagttctgccagtttctcataaagttcaatgtatacctaccctttaatccaacaattctacttccaggcatttacacaaaagaaatgaaaacatgtcatcaaaaatatttgcactcaaataaatggtcacagtgtcttatgcctgagagccagccccatactggaagcaacctaaatatccatcagcaggaagatgcaaaaataatactatagctatgcaatgaaagactactcagcaataaaaagaaacaaactcgtcatacatgcagcaacatgtatgtgtctttttctttctttctttttttttttttttgagacagagttttgttcttgttgcccaggctggagtgcagtggtacgatctcagctcactgcaacctccaccttccagtttgaaactattctcctgcctcagcctcccaagtagctaggattacaagtgcccaccaccacgcccagttaatttttgtattttttagtagagccggggtttcaccatgttggccagactgttctcaaactcctgacctygtgatccacccacctcagccttccaaagtgctgggattacaggcgtgagccaccgcgcccagccaacatgtacatgtctttagaaaagatgcaaaagagtagtgccacatgattccattaagttctagaacagccaaaattaatccatggtagaagatatcagaacagaggagggacgatgatgactgggacaagatagggtgttgtttcttgagtgatgagactgttctatctcttggtagggctgttcgtaacacaggtgtcaaaattcaagaactgtaaacacttaagacctgtgcattccactgaatataaaccatacctcaattaaaaagaggaaaaattaagaaatttttaagcatgaacattgaacaataaataaatatataacattaatgagtgtcccataaattagttagtgttaattagaaaaccccactgtgttttctcagaatttatagtaaccataatcacaataatagctacaaactttctcacctttaaatgtcatcacaactttaggaaacaggttatatcttatattattattattattattattattattattattattattttacattttaacatctctgaaacagggatgtgtcctacaatcaccatcagccaggaagtggtcaggatgcagtggttataactggattattttccctggcctggctgaccatctgcaaccccctggtgtttcaatgtacaaactattttaggaccatctgcgtactggctgttgcctgaaaatcttgtgttgatgtggatttgtaagactgggaaagcaccagcattcaaaacttgaagaaaggtgttagcagtttggcagacaatcctacagaaaactctttattcttgggggagaaaaagttgcctcccatcaacttcaatggttcacaggatgatgacgtgtggggaaacacaaacctcgacaacattgagtccaaatgtgtttcggaagactcagaatgaagaagaagatgccaaggaaaaccttaaccaatttattttcctttttaatgagtgatatgtgatttttcaaaagcctaaatacatctaaaagagctatatcagttaagtataagataaaaattctaaaccatagaaagcactgttactgcttaattgacagcaatttttctttctttgcaatgtataaaataatgggtgctttttatgttcactgaaattcatattgttgtctccttttacagctgggaaagcggaaatctggagaactaaaatatcttgctgaagatcacaatgcagtaaagcatagggcaagtttccaactcgaacatcaccccagaacacttccttttaaacataaattatgttgctaacttttttattgttacttagcaggcagatttcaaactgatacaaatgttgaaaatgatacaaaatgttttcccaccttaaaggcattctgaaacctagcacataacatctgtcactgaagtccatagtttccaatgccatgattggataaagaccctattgagactcatgtgtttgtctctcactgcttaaaacactcatgtgttttaagcaacatgacaagcacaaaagagaacaagtactgctcctgctctccaggaagttaccataaaataaaggggataaggtgccttccaaagaaaaaccaaatactaggagaggtcagaggagaagagagattattccagaaggagagactgaggcagatttcaggagggaagaatataagagtcccaaaagaagaaacaggaacccaggttagagagcaatgggcaaggggtaggaggtgagactaaactcacaaggaagttgcttcaaaacaatagtgccccaaatgccaagctgatgggttggacccatttaggaaaaacccacaaagtccacaggagagcagactgcacttccatgagcttcaagctgtaaacagtaacctcaaataaaacttttcccggatcggtaatttccatcctgaacaatggccaagtcaaggaagctgaggatcgagccccctaccgcatgccacaaagaacctcaagggaccccatgctctggggagctcgcaggaggaccagcacagaggtgtgactgcgttggggcagggtggccacatgtacaatgttgtaccttccaatgttcctccttaggctgtgcttatgaagtcccaacatgtcttctgggctgtgttttcaaccaagagagttaataaaaagtgggatgggcaccaactatgcctccagagacatcaggcagaatatgccacagcgtgccatgtcctcctgcaaacagtcagaaagaaaagatctctaaaagcaaattggaagagactatatatacagagcgagtgccggtacagaagttttgctgcaaatacaccctctaatcccttgcaacccactgcaagctcgagagaccagtgcccttggtaaaacacaggagagatcattaattaaagcctaggccctttctttgatcctgagggataaggcttgggaaaatgcacagtgataaaagtgaacattttaaaagagcagagtgagtgtgaattcccaaagagaccattcatatcaagacccaagccatgagaaaatgctacaatgcccttgattaatactttangaggcattttgacctcagatctaatcccatgaggcttagaccaagaccttgcttgatgcagacctctctgactgctgaaattggtcaggagctcagtgatgaaaatttggaaagtcaatgctgatacctgaagaaagtcccatcagagatctgtgacctgaatcttcataactttcacaggccctttctaaatgccaaggaggatgtgtgtggagaagaatatgccacctatggcatcccaatatgcctgtgcccgcctgctcttcaacaagggaatgaaacctgggtattctcataggttttttaaagcacttgtccacatattttattccgcaaataacacaatttaaaattcagctagaatttgccattttctgtcagggttgtgcatttcttccagtcaccatgacttcctgagatagccaaaacacaaaatttaaattgtgtttaaattaaataaacacaatttatgctgaagctgactttacccactctgttccaacattacttatcctcagcatctctgaattcaaatatcagccaggtgtggtggctcatacctgtaagcccagcactttggcaagccaagatgggaaggttgcttgaggagttggagaccagcctgggcaacatagcaagacccccatctctacaaaaaaattaaaaaattagccaggcatggtggtatgcacctttagtcctagctacttgggaggctgaggtgagaagatcacttgagccgaggagcttgaggtggcagtgagtcatgatcataccactgcactgtagcctgagcaacagagcaagaccccatcccttaaaaaaaatacctgccctctgtacttcatagcacctggggcattttgaacagttcttccttcacctgaggtcattatttgagaatccaattttagagactacaatgctctcattacttggattgtatcatttaatcttcccaacaatccaggaaggggagtctcttaatgattgtccttgatttccagttaaaaaatctagacccagaggtaaccttgtgaaggtcacagagtttcagagttctaagatcccatgcggagattgttgaacttgaacttgtttcttctgatctcatgtcaataagtgacttagtcaacacctgggtagaaatcatcactaaagtgagaaagcacatggtgagggggatgaatcctgtgcccacatggtccctttcacctgcccaatcctcaggcctctcacctggggtacccctcacacacatggcactttcctgaactcatggacatttcagtttgtaacgatcctcctaagattatctactttcacctatccacatttgaactatacttctttagtggaaaaaaagaaataaagccagtgctgagaatgatcaaactccagaaatagtagactgctgtataatttgtcttgcgtgacaccagatctttcaaggaactgcaagctcaagtgcacagttaagcaagatcaagatctacctttgtctttcagagccttcccttggcgaggtcaaggatatctgctgtgagcctgggctggctgaaccaaagggcaatggaggcggccaagaagccccagccaactgctcccctccccaacccccttctccctggaggtcccttcagctagaatttgccattttctgtcagggttgtgcatttcttccagtcaccatgacttcctgagatagccaaaagcatttgctgtgagcagagccccatggctggctttgcacatcaaacatttcagacagccatatatttcatagaatacaaggggaagataacactggacccacagaactttccaacagcttgagaaaatgtggtgcctggtgactgactaaatatacaatcggacaacagccaggccatagcaaaagatggaactctgatccacaaggtgcagcaactttcccagaaaacaatacccttacccacaaaaagcaaccccggaagctggcctgctctaaatcagacctgctggaagccaaattgctctctagtaactatccaggaagctaaataataacttctgtaacaattgactcaaaatgttcaggactggattaataactgatggcttccctaatttttgtccccgattccaacttaggacccaccagagaaagccaaatatggtcccctgaccaagcacacaggatgttcgcttctagtgagccgcctccagcttccccgcattgataacctccaatcagggcacacctgaagccttcgcctttttccactataaagctctcccactcgtctgcccacctttgagtctctgccaaattgcaaatgatggtgtctgactcccttgctgtagcaagctctgcataatagcttgtgcttattctctttgggtaggtcttcatttcttttcacaccagataggcatcatctttgaaaacatctttactgggtctcttgtctccctcctgcagccactgatctaaatcacagacttttttagaaagagggaaaatagtaaagaaagaaagcgattgagttgatgctcccagccactgtctgtaccctgaattttgtttttctgccatatgtcaggtggcctctcctggaccatcgtcactgtttctcctgcttccctgcagctgcagacacaggagcactgtgtaggtctggccttgagcacccacccacccagtacagagccagcccctgctgttctgaatcagtgcagtgaccacttagaacacagtgcccatgcctctgtgatctgaactgctctttccagttccatacaatgttaagaacccacttaagaatgttttaatcattcagtagtttttcgtggtatataataaaaaagtgatttaaatacaatctattttattttagattatatcattaggggttattttacaaagttacctttaaatactattgtaaataaaattaataacaaa

SEQ ID NO: 4 - DNA, 373 bp, Homo sapiens
tttttccagtttcagcaaccctcccaaaaatctgcttgcctcagaaagggaagtttggtagttctggtgcatatggccaattctcatttaaacgagtcattatcctcagagcagtttgcccatgagcagggttaatttataaaatcttgtctcctcattctctacagaaagaaatcgaagctctccaagcaaggatgtttgtgctggaagccaaagatcaacagctgagaagggaaatagaggagcaagagcagcaactccagtggcagggctgcgacctgaccccactggtgggccagctgtccctgggtcagctgcaggaggtcagcaaggccttgcaggacaccctggcctcagccggtcagattccctt

SEQ ID NO: 5 - DNA, 10 bp, Homo sapiens
ggaaggagca

SEQ ID NO: 6 - DNA, 20 bp, Homo sapiens
caccgcgcaggtaggggagc

SEQ ID NO: 7 - DNA, 20 bp, Homo sapiens
ttcttcccaggcagccggga

SEQ ID NO: 8 - DNA, 20 bp, Homo sapiens
atggaggtcagtgtctcttc

SEQ ID NO: 9 - DNA, 20 bp, Homo sapiens
accaacataggtaatatcct

SEQ ID NO: 10 - DNA, 20 bp, Homo sapiens
tatgataaaggtgagtttta

SEQ ID NO: 11 - DNA, 20 bp, Homo sapiens
gggtttccagctgagacgtt

SEQ ID NO: 12 - DNA, 20 bp, Homo sapiens
ccactcagcagagagtactt

SEQ ID NO: 13 - DNA, 20 bp, Homo sapiens
ttgttttaagggccagcgga

SEQ ID NO: 14 - DNA, 20 bp, Homo sapiens
gcagctacaggtgagcaggt

SEQ ID NO: 15 - DNA, 20 bp, Homo sapiens
ttctctacagaaagaaatcg

SEQ ID NO: 16 - DNA, 20 bp, Homo sapiens
ccataaggaggtactgctga

SEQ ID NO: 17 - DNA, 20 bp, Homo sapiens
attcttccagcctccaggaa

SEQ ID NO: 18 - DNA, 20 bp, Homo sapiens
cactactaaggtaagtacct

SEQ ID NO: 19 - DNA, 20 bp, Homo sapiens
ctccccctaggtgtgtatga

SEQ ID NO: 20 - DNA, 20 bp, Homo sapiens
gccatatcaggtaactggca

SEQ ID NO: 21 - DNA, 20 bp, Homo sapiens
cgtgctgtaggaaaccattt

SEQ ID NO: 22 - DNA, 20 bp, Homo sapiens
actgcctatggtaggtagtg

SEQ ID NO: 23 - DNA, 20 bp, Homo sapiens
tttcccccagaaacaagtgt

SEQ ID NO: 24 - DNA, 20 bp, Homo sapiens
aactgtgcaggtaaggataa

SEQ ID NO: 25 - DNA, 20 bp, Homo sapiens
tctgtctcagctgcaagtgt

SEQ ID NO: 26 - DNA, 20 bp, Homo sapiens
ccctttgaaggtattggaag

SEQ ID NO: 27 - DNA, 20 bp, Homo sapiens
tctgtctcagctgcaagtgt

SEQ ID NO: 28 - DNA, 20 bp, Homo sapiens
acagaaagaggtctgtcctt

SEQ ID NO: 29 - DNA, 20 bp, Homo sapiens
ctctcgccaggaatcttaca

SEQ ID NO: 30 - DNA, 20 bp, Homo sapiens
tctcattcatatccttttca

SEQ ID NO: 31 - DNA, 20 bp, Homo sapiens
gtgcccacagtctctcagga

SEQ ID NO: 32 - DNA, 10 bp, Homo sapiens
tatccaaggc

SEQ ID NO: 33 - DNA, 21 bp, Homo sapiens
ggctaaagacctcaccgagga

SEQ ID NO: 34 - DNA, 21 bp, Homo sapiens
aggctggccagtttctctaat

SEQ ID NO: 35 - DNA, 21 bp, Homo sapiens
gcttatccagagcctacagct

SEQ ID NO: 36 - DNA, 21 bp, Homo sapiens
gtcattcttgggaatgtcttc

SEQ ID NO: 37 - DNA, 21 bp, Homo sapiens
gtgttccctacggctttatct

SEQ ID NO: 38 - DNA, 21 bp, Homo sapiens
accacaagccataactcatct

SEQ ID NO: 39 - DNA, 21 bp, Homo sapiens
tgtgagccaccgcacccggcc

SEQ ID NO: 40 - DNA, 21 bp, Homo sapiens
gccctttacagtcatatccta

SEQ ID NO: 41 - DNA, 21 bp, Homo sapiens
ctggtaaaatgtgaagtaata

SEQ ID NO: 42 - DNA, 20 bp, Homo sapiens
ctgagatcccgagacaggct

SEQ ID NO: 43 - DNA, 21 bp, Homo sapiens
agaaagaaatcgaagctctcc

SEQ ID NO: 44 - DNA, 23 bp, Homo sapiens
cattagctgctgctagatcttcc

SEQ ID NO: 45 - DNA, 23 bp, Homo sapiens
taaggcacagaacattctgcctg

SEQ ID NO: 46 - DNA, 24 bp, Homo sapiens
ttattcaattgtgactgcagttgc

SEQ ID NO: 47 - DNA, 21 bp, Homo sapiens
cacctgctctttcactgatgg

SEQ ID NO: 48 - DNA, 25 bp, Homo sapiens
ggtatgatgaaacatcactgagttc

SEQ ID NO: 49 - DNA, 25 bp, Homo sapiens
ctaagtcatccatctgcctctcatc

SEQ ID NO: 50 - DNA, 20 bp, Homo sapiens
gagcctacagctccaggaag

SEQ ID NO: 51 - DNA, 20 bp, Homo sapiens
gagcatggtcccaaagcacc

SEQ ID NO: 52 - DNA, 23 bp, Homo sapiens
gaagcttccctttgtgttctgtc

SEQ ID NO: 53 - DNA, 24 bp, Homo sapiens
cggcagcatctatttgttgccatc

SEQ ID NO: 54 - DNA, 19 bp, Homo sapiens
gcagagggccacgatcacc

SEQ ID NO: 55 - DNA, 20 bp, Homo sapiens
catcccgtcactcctcaggc

SEQ ID NO: 56 - DNA, 23 bp, Homo sapiens
catgacagctggtgtccacgaag

SEQ ID NO: 57 - DNA, 26 bp, Homo sapiens
caaatcagtctctctctccatattcc

SEQ ID NO: 58 - DNA, 23 bp, Homo sapiens
cagtgtgaaactgaggagtctgc

SEQ ID NO: 59 - DNA, 26 bp, Homo sapiens
cgtgtaacaggtatgatgacagagtc

SEQ ID NO: 60 - DNA, 21 bp, Homo sapiens
catttgccttctgctgcaacg

SEQ ID NO: 61 - DNA, 26 bp, Homo sapiens
ctttcttcagatgcaatcattgccac

SEQ ID NO: 62 - DNA, 22 bp, Homo sapiens
caacctcccagtgatatgccac

SEQ ID NO: 63 - DNA, 23 bp, Homo sapiens
ctgccttactgagcactgcactg

SEQ ID NO: 64 - DNA, 25 bp, Homo sapiens
cagtaacacaatacagtactcaggc

SEQ ID NO: 65 - DNA, 24 bp, Homo sapiens
ctgcactgtgttactgagcattgc

SEQ ID NO: 66 - DNA, 23 bp, Homo sapiens
ctcaggcagtgcaatactcagtg

SEQ ID NO: 67 - DNA, 23 bp, Homo sapiens
actagctgccctgtgttactgag

SEQ ID NO: 68 - DNA, 22 bp, Homo sapiens
gtgtagtgctcagtaggacagc

SEQ ID NO: 69 - DNA, 23 bp, Homo sapiens
gtacttgaccagagagggtactc

SEQ ID NO: 70 - DNA, 22 bp, Homo sapiens
caaagtgcactgaggaaggtgg

SEQ ID NO: 71 - DNA, 26 bp, Homo sapiens
gtgatgtaaatcagagtttggactgg

SEQ ID NO: 72 - DNA, 26 bp, Homo sapiens
ctcctattcatatcccatagatctag

SEQ ID NO: 73 - DNA, 21 bp, Homo sapiens
cagaagtgctgcacagcatgg

SEQ ID NO: 74 - DNA, 23 bp, Homo sapiens
cactggcgtttccagaaggcatc

SEQ ID NO: 75 - DNA, 24 bp, Homo sapiens
cactggaattaactcaaggattcc

SEQ ID NO: 76 - DNA, 27 bp, Homo sapiens
cattttcaatgatgaaccaaattcctg

SEQ ID NO: 77 - DNA, 25 bp, Homo sapiens
gcagcagggaaatgaacacactatc

SEQ ID NO: 78 - DNA, 27 bp, Homo sapiens
ctgcaagttaataactgccttgaattg

SEQ ID NO: 79 - DNA, 26 bp, Homo sapiens
ctgctgtatagtatgattgaggatac

SEQ ID NO: 80 - DNA, 23 bp, Homo sapiens
caaccttctcaggacaagcccac

SEQ ID NO: 81 - DNA, 24 bp, Homo sapiens
gtgaccatttgaaagccagcattc

SEQ ID NO: 82 - DNA, 25 bp, Homo sapiens
gttagaatctgatttgactgggatg

SEQ ID NO: 83 - DNA, 21 bp, Homo sapiens
cttgggaggctgaggcagtag

SEQ ID NO: 84 - DNA, 25 bp, Homo sapiens
gcacatactggaaatgatgagttag

SEQ ID NO: 85 - DNA, 23 bp, Homo sapiens
atgcctgtaaccccagctacttg

SEQ ID NO: 86 - DNA, 21 bp, Homo sapiens
ctctacctcccaggttcaagc

SEQ ID NO: 87 - DNA, 25 bp, Homo sapiens
ggtaccaaccgttacatgtttctgg

SEQ ID NO: 88 - DNA, 19 bp, Homo sapiens
gcacccggccaactttctg

SEQ ID NO: 89 - DNA, 25 bp, Homo sapiens
ctctgactgttaggttcactattac

SEQ ID NO: 90 - DNA, 24 bp, Homo sapiens
ctaaggcacagagctggtaaaatg

SEQ ID NO: 91 - DNA, 21 bp, Homo sapiens
gatgatggtggggtgaatagg

SEQ ID NO: 92 - DNA, 22 bp, Homo sapiens
agcctcactgtgaagtctaggc

SEQ ID NO: 93 - DNA, 23 bp, Homo sapiens
ctatcatccatacttcaaaggac

SEQ ID NO: 94 - DNA, 23 bp, Homo sapiens
cactgccttctgatttttagctg

SEQ ID NO: 95 - DNA, 23 bp, Homo sapiens
gaggacaaaacacgatgtgctgg

SEQ ID NO: 96 - DNA, 23 bp, Homo sapiens
ctcgctgaggagaagaaaggagc

SEQ ID NO: 97 - DNA, 19 bp, Homo sapiens
ggcacgccctgacctacgc

SEQ ID NO: 98 - DNA, 27 bp, Homo sapiens
ctgtagtggtattgaattgtggttacc

SEQ ID NO: 99 - DNA, 22 bp, Homo sapiens
cagaagcactctctcctggctc

SEQ ID NO: 100 - DNA, 23 bp, Homo sapiens
gccaatgctggaaaggaaacagc

SEQ ID NO: 101 - DNA, 18 bp, Homo sapiens
tgccagctcctcccgctc

SEQ ID NO: 102 - DNA, 23 bp, Homo sapiens
catccctccatcttctccatcac

SEQ ID NO: 103 - DNA, 18 bp, Homo sapiens
cacgctgctggagcgggc

SEQ ID NO: 104 - DNA, 23 bp, Homo sapiens
gagtcagttgcctaggagcttgc

SEQ ID NO: 105 - DNA, 21 bp, Homo sapiens
gtgcaggaagcctccaggatg

SEQ ID NO: 106 - DNA, 24 bp, Homo sapiens
cctatccctgaaccattgcaagag

SEQ ID NO: 107 - DNA, 20 bp, Homo sapiens
cagcagctccgggctggttc

SEQ ID NO: 108 - DNA, 22 bp, Homo sapiens
ctgggccagtaagatctgcatg

SEQ ID NO: 109 - DNA, 21 bp, Homo sapiens
gaaacagggctccggaccaag

SEQ ID NO: 110 - DNA, 22 bp, Homo sapiens
ccagacagtgtggccttgactc

SEQ ID NO: 111 - DNA, 23 bp, Homo sapiens
ggctgcagctgttgctactcaac

SEQ ID NO: 112 - DNA, 20 bp, Homo sapiens
ctgcagcgatggagcaaggc

SEQ ID NO: 113 - DNA, 20 bp, Homo sapiens
acatcgcgggtcctcgtgag

SEQ ID NO: 114 - DNA, 21 bp, Homo sapiens
cagagaggctgagtcccattg

SEQ ID NO: 115 - DNA, 22 bp, Homo sapiens
gcactggctcccatttcctgag

SEQ ID NO: 116 - DNA, 21 bp, Homo sapiens
ccagttctctggatccctcac

SEQ ID NO: 117 - DNA, 24 bp, Homo sapiens
gaggaagtcagttgagcccagaac

SEQ ID NO: 118 - DNA, 27 bp, Homo sapiens
cagtttctaaatgttcttagttttcac

SEQ ID NO: 119 - DNA, 28 bp, Homo sapiens
gatggaaagaaaattgggacatgatgac

SEQ ID NO: 120 - DNA, 24 bp, Homo sapiens
gttcactacaactggagctaagag

SEQ ID NO: 121 - DNA, 23 bp, Homo sapiens
ctatgtgggagctgagaggtagg

SEQ ID NO: 122 - DNA, 25 bp, Homo sapiens
catgaggatttcagcttctgcatac

SEQ ID NO: 123 - DNA, 22 bp, Homo sapiens
gagctatgattgcaccactgcc

SEQ ID NO: 124 - DNA, 19 bp, Homo sapiens
cgacctgaccccactggtg

SEQ ID NO: 125 - DNA, 20 bp, Homo sapiens
gactgccccacaccgtgaag

SEQ ID NO: 126 - DNA, 25 bp, Homo sapiens
gtttggtagttctggtgcatatggc

SEQ ID NO: 127 - DNA, 20 bp, Homo sapiens
gcatggaagggaatctgacc